(19) United States
(12) Patent Application Publication
(10) Pub. No.: US 2022/0213099 A1
(43) Pub. Date: Jul. 7, 2022

(54) PRODRUG COMPOUNDS

(71) Applicant: BRISTOL-MYERS SQUIBB COMPANY, Princeton, NJ (US)

(72) Inventors: Alaric J. DYCKMAN, Lawrenceville, NJ (US); Murugaiah Andappan Murugaiah Subbaiah, Bangalore (IN)

(21) Appl. No.: 17/609,021

(22) PCT Filed: May 6, 2020
(86) PCT No.: PCT/US2020/031558

§ 371 Date: Nov. 5, 2021

Related U.S. Application Data
(60) Provisional application No. 62/844,439, filed on May 7, 2019.

(51) Int. Cl. C07D 471/04; C07F 9/09

(52) U.S. Cl. CPC C07D 471/04; C07F 9/09

(57) ABSTRACT

Disclosed are compounds of Formula (I), Formula (II), Formula (III), and Formula (IV): or salts thereof, wherein R₁, R₂, R₃, and R₄ are defined herein. Also disclosed are methods of using the compounds as prodrugs of an inhibitor of signaling through Toll-like receptor 7, or 8, or 9, and pharmaceutical compositions comprising such compounds. These prodrugs compounds are useful in treating inflammatory and autoimmune diseases.

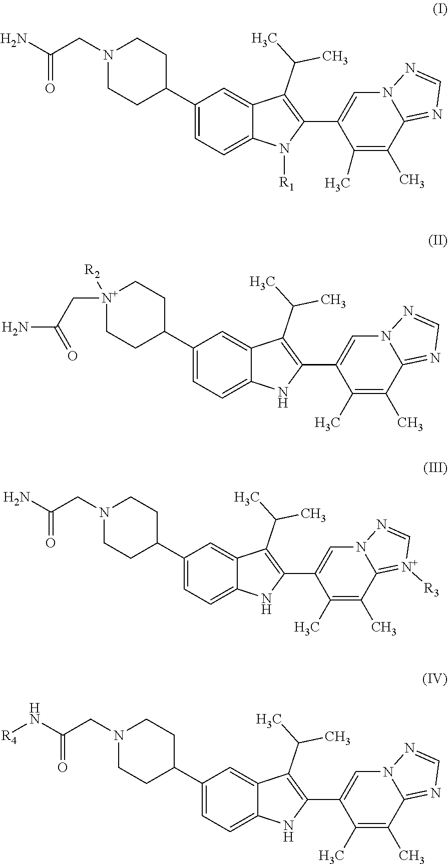

### CROSS-REFERENCE TO RELATED APPLICATIONS

This application claims the benefit of U.S. Provisional Application No. 62/844,439, filed May 7, 2019, the contents of which are specifically incorporated by reference herein.

### DESCRIPTION

The present invention generally relates to substituted indole compounds useful as prodrugs of inhibitors of signaling through Toll-like receptor 7, 8, or 9 (TLR7, TLR8, TLR9) or combinations thereof. Provided herein are prodrug compounds, compositions comprising such compounds, and methods of their use. The invention further pertains to pharmaceutical compositions containing at least one compound according to the invention that are useful for the treatment of conditions related to TLR modulation, such as inflammatory and autoimmune diseases, and methods of inhibiting the activity of TLRs in a mammal.

Toll/IL-1 receptor family members are important regulators of inflammation and host resistance. The Toll-like receptor family recognizes molecular patterns derived from infectious organisms including bacteria, fungi, parasites, and viruses (reviewed in Kawai, T. et al., Nature Immunol., 11:373-384 (2010)). Ligand binding to the receptor induces dimerization and recruitment of adaptor molecules to a conserved cytoplasmic motif in the receptor termed the Toll/IL-1 receptor (TIR) domain with the exception of TLR3, all TLRs recruit the adaptor molecule MyD88. The IL-1 receptor family also contains a cytoplasmic TIR motif and recruits MyD88 upon ligand binding (reviewed in Sims, J. E. et al., Nature Rev. Immunol., 10:89-102 (2010)).

Toll-like receptors (TLRs) are a family of evolutionarily conserved, transmembrane innate immune receptors that participate in the first-line defense. As pattern recognition receptors, the TLRs protect against foreign molecules, activated by pathogen associated molecular patterns (PAMPs), or from damaged tissue, activated by danger associated molecular patterns (DAMPs). A total of 13 TLR family members have been identified, 10 in human, that span either the cell surface or the endosomal compartment. TLR7-9 are among the set that are endosomally located and respond to single-stranded RNA (TLR7 and TLR8) or unmethylated single-stranded DNA containing cytosine-phosphate-guanine (CpG) motifs (TLR9).

Activation of TLR7/8/9 can initiate a variety of inflammatory responses (cytokine production, B cell activation and IgG production, Type I interferon response). In the case of autoimmune disorders, the aberrant sustained activation of TLR7/8/9 leads to worsening of disease states. Whereas overexpression of TLR7 in mice has been shown to exacerbate autoimmune disease, knockout of TLR7 in mice was found to be protective against disease in lupus-prone MRL/lpr mice. Dual knockout of TLR7 and 9 showed further enhanced protection.

As numerous conditions may benefit by treatment involving modulation of cytokines, IFN production and B cell activity, it is immediately apparent that compounds capable of modulating TLR7 and/or TLR8 and/or TLR9 and methods of using these compounds could provide substantial therapeutic benefits to a wide variety of patients.

U.S. Pat. No. 10,071,079 B2 discloses substituted indole compounds useful treating inflammatory and autoimmune diseases, such as lupus. This patent discloses the compound 2-(4-(2-(7,8-dimethyl-[1,2,4]triazolo[1,5-a]pyridin-6-yl)-3-isopropyl-1H-indol-5-yl)piperidin-1-yl)acetamide, having the structure of Formula (A):

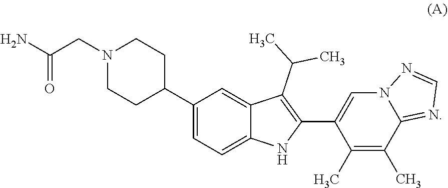 [SMILES: Cc1c(-c2[nH]c3ccc(C4CCN(CC(N)=O)CC4)cc3c2C(C)C)cn2ncnc2c1C]

as Example 15. The compound has activity as an inhibitor of TLR7 and/or TLR8, thus making it useful for treating inflammatory and autoimmune disease. The preparation process and method of using the compound are also disclosed in U.S. Pat. No. 10,071,079 B2. This patent is assigned to the present assignee and is incorporated herein by reference in its entirety.

The usefulness of an oral formulation, however, requires that the active agent be bioavailable and that the level of bioavailability does not vary widely. The bioavailability of orally administered drugs is often affected by various factors including, for example, the solubility of the drug in the gastrointestinal tract, the stability of the drug in the gastrointestinal tract, and drug absorption in the gastrointestinal tract. Further, these factors may be affected by co-administration of other drugs and/or the intake of food, which may lead to variability in the bioavailability of orally administered drug.

The aqueous solubility of Compound A is dependent on the pH of the aqueous medium. Compound A has higher solubility at pH of 1 than at pH of 4 or pH of 6. In the oral administration of Compound A, the solubility and hence the bioavailability of Compound A can be affected by the pH of the stomach contents. The normal pH of the stomach is 1.2 to 1.8 according to C. J. Perigard, Clinical Analysis, Chapter 32, in Remington: The Science and Practice of Pharmacy 20^{th} Edition, A. R. Gennaro, editor; 2000, Lippinocott Williams & Wilkins, Baltimore, Md. However, patients often take other medications to treat medical conditions related or unrelated to the treatment of inflammatory and autoimmune diseases with Compound A. For example, medications such as antacids or proton pump inhibitors can raise the pH of the stomach.

As may be appreciated, there remains a need for improved delivery of Compound A to the patient.

### SUMMARY OF THE INVENTION

Applicants have found prodrugs of Compound A useful for the delivery of Compound A to a patient. The prodrugs were found to have greater solubility at pH 4 and/or pH 6.5 than Compound A. By improving solubility over a broader pH range, the compounds of the invention would have less variation in solubility in the stomach acid levels of the patient. Variations in the stomach pH may occur because of other medications or food that has been ingested. It is expected that absorption of the prodrugs would be less susceptible to variations in the pH of the stomach and other parts of the gastrointestinal tract, and thus, would be absorbed more uniformly independently of the pH of the stomach.

The present invention fills the foregoing need by providing prodrug compounds of 2-(4-(2-(7,8-dimethyl-[1,2,4]triazolo[1,5-a]pyridin-6-yl)-3-isopropyl-1H-indol-5-yl) piperidin-1-yl)acetamide, which is useful as an inhibitor of TLR7 and/or TLR8, including salts of the prodrug compounds.

The present invention provides prodrug compounds of Formula (I), Formula (II), Formula (III), and Formula (IV) that are useful as inhibitors of signaling through Toll-like receptor 7, 8, or 9 and are useful for the treatment of proliferative diseases, allergic diseases, autoimmune diseases and inflammatory diseases, or pharmaceutically acceptable salts or solvates thereof.

The present invention also provides pharmaceutical compositions comprising a pharmaceutically acceptable carrier and at least one of the compounds of the present invention or pharmaceutically acceptable salts or solvates thereof.

The present invention also provides a method for inhibition of Toll-like receptor 7, 8, or 9 comprising administering to a host in need of such treatment a therapeutically effective amount of at least one of the compounds of the present invention or pharmaceutically acceptable salts or solvates thereof.

The present invention also provides a method for treating proliferative, metabolic, allergic, autoimmune and inflammatory diseases, comprising administering to a host in need of such treatment a therapeutically effective amount of at least one of the compounds of the present invention or pharmaceutically acceptable salts or solvates thereof.

The present invention also provides a method of treating a disease or disorder associated with Toll-like receptor 7, 8, or 9 activity, the method comprising administering to a mammal in need thereof, at least one of the compounds of Formula (I), Formula (II), Formula (III), and Formula (IV), or pharmaceutically acceptable salts or solvates thereof.

The present invention also provides processes and intermediates for making the compounds of Formula (I), Formula (II), Formula (III), and Formula (IV), including salts and solvates thereof.

The present invention also provides at least one of the compounds of Formula (I), Formula (II), Formula (III), and Formula (IV), or pharmaceutically acceptable salts or solvates thereof, for use in therapy.

The present invention also provides the use of at least one of the compounds of Formula (I), Formula (II), Formula (III), and Formula (IV), or pharmaceutically acceptable salts or solvates thereof, for the manufacture of a medicament for the treatment of prophylaxis of Toll-like receptor 7, 8, or 9 related conditions, such as allergic disease, autoimmune diseases, inflammatory diseases, and proliferative diseases.

The at least one compound of Formula (I), Formula (II), Formula (III), and Formula (IV), and compositions comprising the compounds of Formula (I), Formula (II), Formula (III), and Formula (IV), may be used in treating, preventing, or curing various Toll-like receptor 7, 8, or 9 related conditions. Pharmaceutical compositions comprising these compounds are useful for treating, preventing, or slowing the progression of diseases or disorders in a variety of therapeutic areas, such as allergic disease, autoimmune diseases, inflammatory diseases, and proliferative diseases.

These and other features of the invention will be set forth in expanded form as the disclosure continues.

### DETAILED DESCRIPTION

The first aspect of the present invention provides at least one compound of Formula (I), Formula (II), Formula (III), and Formula (IV):

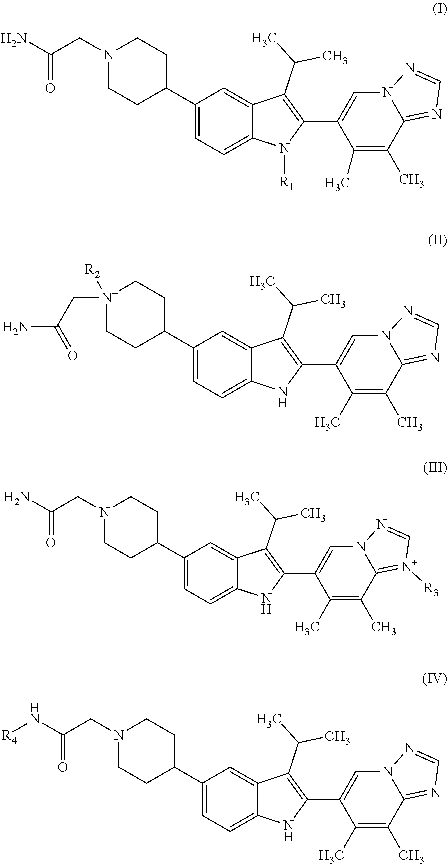

or salts thereof, wherein:
R₁ is —CH₂OH, —C(O)O(C₁₋₄ alkyl), —C(O)CH₂NRₓRₓ, —C(O)(CH₂)₁₋₃OP(O)(OH)₂, —C(O)CH₂NRₓC(O)OCH₂OP(O)(OH)₂, —C(O)OCH₂(pyrrolidinyl), —C(O)OCH₂(piperidinyl), —C(O)OCHRₓOC(O)(aminocyclopropyl), —C(O)OCH(CH₃)OC(O)(aminocyclopropyl), —C(O)OCH₂OP(O)(OH)₂, —P(O)(OH)₂, —SCH₂CH(NH₂)C(O)OH,

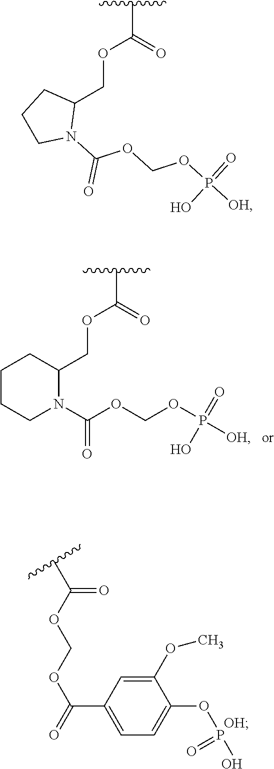

R₂ and R₃ are independently —CH₂OP(O)(OH)₂, —CH₂OC(O)NRₓCH₂CH₂NRₓRₓ, —CH₂OC(O)NRₓCH₂CH₂OP(O)(OH)₂, or

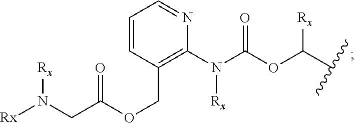

and
R₄ is —P(O)(OH)₂; and
each Rₓ is independently hydrogen or —CH₃.

One embodiment provides at least one compound of Formula (I), Formula (II), Formula (III), and Formula (IV) or salts thereof, wherein:
R₁ is —CH₂OH, —C(O)CH₂NH(CH₃), —C(O)CH₂CH₂CH₂OP(O)(OH)₂, —C(O)CH₂N(CH₃)C(O)OCH₂OP(O)(OH)₂, —C(O)OCH₂CH₃, —C(O)OCH₂(pyrrolidinyl), —C(O)OCH₂(piperidinyl), —C(O)OCH₂OC(O)(aminocyclopropyl), —C(O)OCH(CH₃)OC(O)(aminocyclopropyl), —C(O)OCH₂OP(O)(OH)₂, —P(O)(OH)₂, —SCH₂CH(NH₂)C(O)OH,

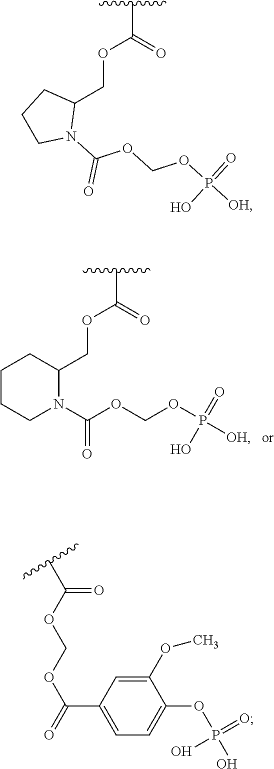 [SMILES: *C(=O)OCC1CCCN1C(=O)OCOP(=O)(O)O ; *C(=O)OCC1CCCCN1C(=O)OCOP(=O)(O)O ; *C(=O)OCOC(=O)c1ccc(OP(=O)(O)O)c(OC)c1]

R₂ is —CH₂OP(O)(OH)₂, —CH₂OC(O)N(CH₃)CH₂CH₂NH(CH₃), —CH₂OC(O)N(CH₃)CH₂CH₂OP(O)(OH)₂,

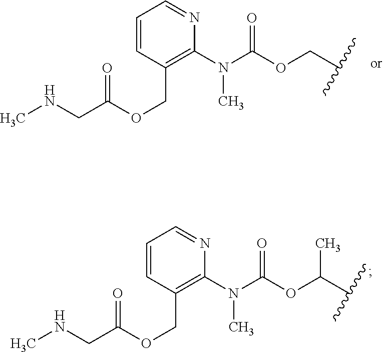 [SMILES: *COC(=O)N(C)c1ncccc1COC(=O)CNC ; *C(C)OC(=O)N(C)c1ncccc1COC(=O)CNC]

R₃ is —CH₂P(O)(OH)₂, —CH₂OC(O)N(CH₃)CH₂CH₂OP(O)(OH)₂, or

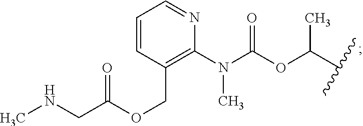 [SMILES: *C(C)OC(=O)N(C)c1ncccc1COC(=O)CNC]

and
R₄ is —P(O)(OH)₂.

One embodiment provides a compound of Formula (I) or a salt thereof, wherein:
R₁ is —CH₂OH, —C(O)O(C₁₋₄ alkyl), —C(O)CH₂NRₓRₓ, —C(O)(CH₂)₁₋₃OP(O)(OH)₂, —C(O)CH₂NRₓC(O)OCH₂OP(O)(OH)₂, —C(O)OCH₂(pyrrolidinyl), —C(O)OCH₂(piperidinyl), —C(O)OCHRₓOC(O)(aminocyclopropyl), —C(O)OCH(CH₃)OC(O)(aminocyclopropyl), —C(O)OCH₂OP(O)(OH)₂, —P(O)(OH)₂, —SCH₂CH(NH₂)C(O)OH,

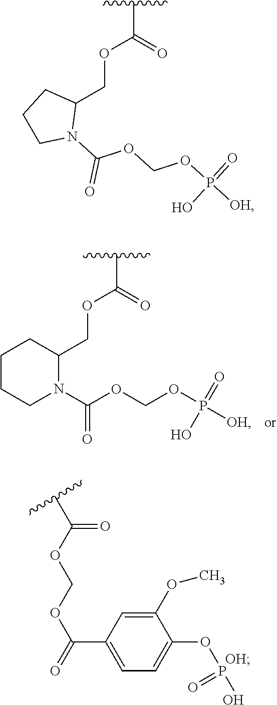

and
each Rₓ is independently hydrogen or —CH₃. Included in this embodiment are compounds in which R₁ is —CH₂OH, —C(O)CH₂NH(CH₃), —C(O)CH₂CH₂CH₂OP(O)(OH)₂, —C(O)CH₂N(CH₃)C(O)OCH₂OP(O)(OH)₂, —C(O)OCH₂CH₃, —C(O)OCH₂(pyrrolidinyl), —C(O)OCH₂(piperidinyl), —C(O)OCH₂OC(O)(aminocyclopropyl), —C(O)OCH(CH₃)OC(O)(aminocyclopropyl), —C(O)OCH₂OP(O)(OH)₂, —P(O)(OH)₂, —SCH₂CH(NH₂)C(O)OH,

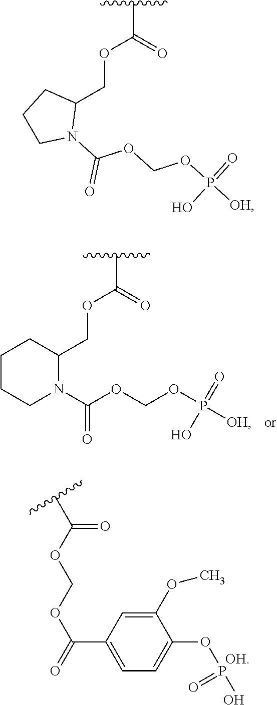

One embodiment provides at least one compound of Formula (II) and Formula (III) or salts thereof, wherein:
R₂ and R₃ are independently —CH₂OP(O)(OH)₂, —CH₂OC(O)NRₓCH₂CH₂NRₓRₓ, —CH₂OC(O)NRₓCH₂CH₂OP(O)(OH)₂, or

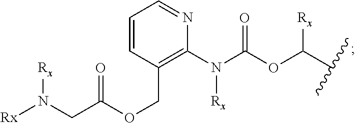

and
each Rₓ is independently hydrogen or —CH₃. Included in this embodiment are compounds in which:
R₂ is —CH₂OP(O)(OH)₂, —CH₂OC(O)N(CH₃)CH₂CH₂NH(CH₃), —CH₂OC(O)N(CH₃)CH₂CH₂OP(O)(OH)₂,

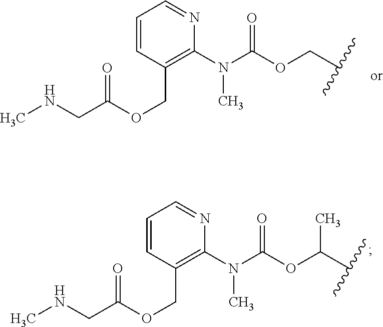 [SMILES: *COC(=O)N(C)c1ncccc1COC(=O)CNC ; *C(C)OC(=O)N(C)c1ncccc1COC(=O)CNC]

R₃ is —CH₂P(O)(OH)₂, —CH₂OC(O)N(CH₃)CH₂CH₂OP(O)(OH)₂, or

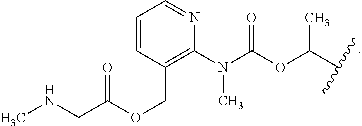 [SMILES: *C(C)OC(=O)N(C)c1ncccc1COC(=O)CNC]

One embodiment provides a compound of Formula (II) or a salt thereof, wherein:
R₂ is —CH₂OP(O)(OH)₂, —CH₂OC(O)NRₓCH₂CH₂NRₓRₓ, —CH₂OC(O)NRₓCH₂CH₂OP(O)(OH)₂, or

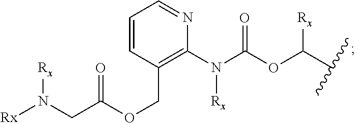

and
each Rₓ is independently hydrogen or —CH₃. Included in this embodiment are compounds in which R₂ is —CH₂OP(O)(OH)₂, —CH₂OC(O)N(CH₃)CH₂CH₂NH(CH₃), —CH₂OC(O)N(CH₃)CH₂CH₂OP(O)(OH)₂,

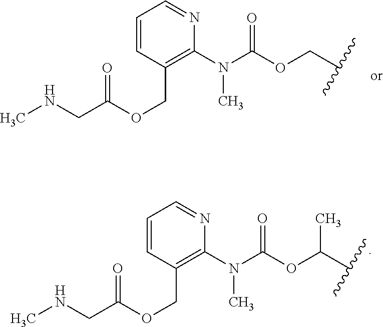 [SMILES: *COC(=O)N(C)c1ncccc1COC(=O)CNC ; *C(C)OC(=O)N(C)c1ncccc1COC(=O)CNC]

One embodiment provides a compound of Formula (III) or a salt thereof, wherein:
R₃ is —CH₂OP(O)(OH)₂, —CH₂OC(O)NRₓCH₂CH₂NRₓRₓ, —CH₂OC(O)NRₓCH₂CH₂OP(O)(OH)₂, or

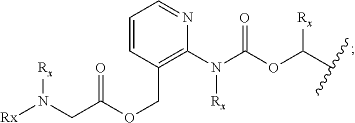

and
each Rₓ is independently hydrogen or —CH₃. Included in this embodiment are compounds in which R₃ is —CH₂P(O)(OH)₂, —CH₂OC(O)N(CH₃)CH₂CH₂OP(O)(OH)₂, or

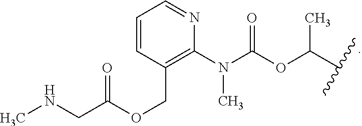 [SMILES: *C(C)OC(=O)N(C)c1ncccc1COC(=O)CNC]

One embodiment provides a compound of Formula (IV) or a salt thereof, wherein
R₄ is —P(O)(OH)₂.

One embodiment provides a compound of Formula (I) or a salt thereof, wherein said compound is: (S)-piperidin-2-ylmethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (1); (S)-(1-(((phosphonooxy)methoxy)carbonyl)piperidin-2-yl)methyl 5-(1-(2-amino-2-oxoethyl) piperidin-4-yl)-2-(7,8-dimethyl-[1,2,4]triazolo[1,5-a]pyridin-6-yl)-3-isopropyl-1H-indole-1-carboxylate (2); (S)-pyrrolidin-2-ylmethyl 5-(1-(2-amino-2-oxoethyl) piperidin-4-yl)-2-(7,8-dimethyl-[1,2,4]triazolo[1,5-a]pyridin-6-yl)-3-isopropyl-1H-indole-1-carboxylate (3); (S)-(1-(((phosphonooxy)methoxy)carbonyl)pyrrolidin-2-yl) methyl 5-(1-(2-amino-2-oxoethyl)piperidin-4-yl)-2-(7,8-dimethyl-[1,2,4]triazolo[1,5-a] pyridin-6-yl)-3-isopropyl-1H-indole-1-carboxylate (4); 2-(4-(2-(7,8-dimethyl-[1,2,4]triazolo[1,5-a]pyridin-6-yl)-3-isopropyl-1-(methylglycyl)-1H-indol-5-yl) piperidin-1-yl)acetamide ditrifluoroacetate (5); 1-((1-aminocyclopropane-1-carbonyl) oxy)ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate ditrifluoroacetate (6-7); 4-(5-(1-(2-amino-2-oxoethyl)piperidin-4-yl)-2-(7,8-dimethyl-[1,2,4]triazolo[1,5-a] pyridin-6-yl)-3-isopropyl-1H-indol-1-yl)-4-oxobutyl dihydrogen phosphate (10); S-(5-(1-(2-amino-2-oxoethyl)piperidin-4-yl)-2-(7,8-dimethyl-[1,2,4]triazolo[1,5-a] pyridin-6-yl)-3-isopropyl-1H-indol-1-yl)-L-cysteine (11); ((1-aminocyclopropane-1-carbonyl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate trifluoroacetate (15); (phosphonooxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (16); (5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)phosphonate (18); (phosphonooxy)methyl (2-(5-(1-(2-amino-2-oxoethyl)piperidin-4-yl)-2-(7,8-dimethyl-[1,2,4]triazolo[1,5-a] pyridin-6-yl)-3-isopropyl-1H-indol-1-yl)-2-oxoethyl)(methyl)carbamate (19); ((3-methoxy-4-(phosphonooxy) benzoyl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (22); ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (24); or 2-(4-(2-(7,8-dimethyl-[1,2,4]triazolo[1,5-a]pyridin-6-yl)-1-(hydroxymethyl)-3-isopropyl-1H-indol-5-yl)piperidin-1-yl)acetamide (25).

One embodiment provides a compound of Formula (II) or a salt thereof, wherein said compound is: 1-(2-amino-2-oxoethyl)-4-(2-(7,8-dimethyl-[1,2,4]triazolo[1,5-a]pyridin-6-yl)-3-isopropyl-1H-indol-5-yl)-1-((phosphonooxy)methyl)piperidin-1-ium trifluoroacetate (8); 1-(2-amino-2-oxoethyl)-4-(2-(7,8-dimethyl-[1,2,4]triazolo[1,5-a]pyridin-6-yl)-3-isopropyl-1H-indol-5-yl)-1-(((methyl(3-(((methylglycyl)oxy)methyl) pyridin-2-yl)carbamoyl)oxy)methyl)piperidin-1-ium ditrifluoroacetate (12); 1-(2-amino-2-oxoethyl)-4-(2-(7,8-dimethyl-[1,2,4]triazolo[1,5-a]pyridin-6-yl)-3-isopropyl-1H-indol-5-yl)-1-(1-((methyl(3-(((methylglycyl)oxy)methyl)pyridin-2-yl)carbamoyl)oxy)ethyl) piperidin-1-ium ditrifluoroacetate (14); 1-(2-amino-2-oxoethyl)-4-(2-(7,8-dimethyl-[1,2,4]triazolo[1,5-a]pyridin-6-yl)-3-isopropyl-1H-indol-5-yl)-1-(((methyl(2-(phosphonooxy)ethyl)carbamoyl)oxy)methyl)piperidin-1-ium trifluoroacetate (20); or 1-(2-amino-2-oxoethyl)-4-(2-(7,8-dimethyl-[1,2,4]triazolo[1,5-a]pyridin-6-yl)-3-isopropyl-1H-indol-5-yl)-1-(((methyl(2-(methylamino)ethyl)carbamoyl)oxy)methyl)piperidin-1-ium trifluoroacetate (23).

One embodiment provides a compound of Formula (III) or a salt thereof, wherein said compound is: 6-(5-(1-(2-amino-2-oxoethyl)piperidin-4-yl)-3-isopropyl-1H-indol-2-yl)-7,8-dimethyl-1-((phosphonooxy) methyl)-[1,2,4]triazolo[1,5-a]pyridin-1-ium trifluoroacetate (9); 6-(5-(1-(2-amino-2-oxoethyl)piperidin-4-yl)-3-isopropyl-1H-indol-2-yl)-7,8-dimethyl-1-(((methyl(3-(((methylglycyl)oxy)methyl)pyridin-2-yl)carbamoyl)oxy) methyl)-[1,2,4]triazolo[1,5-a]pyridin-1-ium tritrifluoroacetate (13); or 6-(5-(1-(2-amino-2-oxoethyl)piperidin-4-yl)-3-isopropyl-1H-indol-2-yl)-7,8-dimethyl-1-(((methyl(2-(phosphonooxy)ethyl)carbamoyl)oxy)methyl)-[1,2,4]triazolo[1,5-a]pyridin-1-ium trifluoroacetate (21).

One embodiment provides a compound of Formula (IV) or a salt thereof, wherein said compound is: 5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)phosphonic acid (17).

The present invention may be embodied in other specific forms without departing from the spirit or essential attributes thereof. The invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

### Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phrase “compounds” refers to at least one compound. For example, at least one compound of Formula (I), Formula (II), Formula (III), and Formula (IV) includes a compound of Formula (II). In another example, at least one compound of Formula (I), Formula (II), Formula (III), and Formula (IV) includes a compound of Formula (I) and a compound of Formula (II).

Unless otherwise indicated, any heteroatom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,

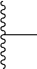

is used in structural formulas herein to depict the bond that is the point of attachment of the moiety or substituent to the core or backbone structure.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₆ alkyl” denotes straight and branched chain alkyl groups with one to six carbon atoms.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I), Formula (II), Formula (III), and/or Formula (IV) can form salts which are also within the scope of this invention. Unless otherwise indicated, reference to an inventive compound is understood to include reference to one or more salts thereof. The term “salt(s)” denotes acidic and/or basic salt(s) formed with inorganic and/or organic acids and bases. In addition, the term “salt(s) may include zwitterions (inner salts), e.g., when a compound of Formula (I), Formula (II), Formula (III), and/or Formula (IV) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of Formula (I), Formula (II), Formula (III), and/or Formula (IV) may be formed, for example, by reacting a compound of the Formula (I), Formula (II), Formula (III), and/or Formula (IV) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-Q-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts. The compounds of Formula (I), Formula (II), Formula (III), and/or Formula (IV) can be provided as amorphous solids or crystalline solids. Lyophilization can be employed to provide the compounds of Formula (I), Formula (II), Formula (III), and/or Formula (IV) as amorphous solids.

It should further be understood that solvates (e.g., hydrates) of the compounds of Formula (I), Formula (II), Formula (III), and/or Formula (IV) are also within the scope of the present invention. The term “solvate” means a physical association of a compound of Formula (I), Formula (II), Formula (III), and/or Formula (IV) with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

In addition, compounds of Formula (I), Formula (II), Formula (III), and/or Formula (IV), subsequent to their preparation, can be isolated and purified to obtain a composition containing an amount by weight equal to or greater than 99% of a compound of Formula (I), Formula (II), Formula (III), and Formula (IV), respectively (“substantially pure”), which is then used or formulated as described herein. Such “substantially pure” compounds of Formula (I), Formula (II), Formula (III), and/or Formula (IV) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

“Therapeutically effective amount” is intended to include an amount of a compound of the present invention alone or an amount of the combination of compounds claimed or an amount of a compound of the present invention in combination with other active ingredients effective to act as an inhibitor to TLR7/8/9, or effective to treat or prevent autoimmune and/or inflammatory disease states, such as SLE, IBD, multiple sclerosis (MS), and Sjögren's syndrome, and rheumatoid arthritis.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD₃.

### Utility

The human immune system has evolved to defend the body from micro-organisms, viruses, and parasites that can cause infection, disease or death. Complex regulatory mechanisms ensure that the various cellular components of the immune system target the foreign substances or organisms, while not causing permanent or significant damage to the individual. While the initiating events are not well understood at this time, in autoimmune disease states the immune system directs its inflammatory response to target organs in the afflicted individual. Different autoimmune diseases are typically characterized by the predominate or initial target organ or tissues affected; such as the joint in the case of rheumatoid arthritis, the thyroid gland in the case of Hashimoto's thyroiditis, the central nervous system in the case of multiple sclerosis, the pancreas in the case of type I diabetes, and the bowel in the case of inflammatory bowel disease.

The compounds of the invention inhibit signaling through Toll-like receptor 7, or 8, or 9 (TLR7, TLR8, TLR9) or combinations thereof. Accordingly, compounds of Formula (I), Formula (II), Formula (III), and Formula (IV) have utility as prodrugs of Compound A, which is useful for treating conditions associated with the inhibition of signaling through one or more of TLR7, TLR8, or TLR9. Such conditions include TLR7, TLR8, or TLR9 receptor associated diseases in which cytokine levels are modulated as a consequence of intracellular signaling.

As used herein, the terms “treating” or “treatment” encompass the treatment of a disease state in a mammal, particularly in a human, and include: (a) preventing or delaying the occurrence of the disease state in a mammal, in particular, when such mammal is predisposed to the disease state but has not yet been diagnosed as having it; (b) inhibiting the disease state, i.e., arresting its development; and/or (c) achieving a full or partial reduction of the symptoms or disease state, and/or alleviating, ameliorating, lessening, or curing the disease or disorder and/or its symptoms.

In view of their activity as selective inhibitors of TLR7, TLR8, or TLR9, compounds of Formula (I), Formula (II), Formula (III), and Formula (IV) are useful as prodrugs of Compound A, which is useful for treating TLR7, TLR8, or TLR9 family receptor associated diseases, but not limited to, inflammatory diseases such as Crohn's disease, ulcerative colitis, asthma, graft versus host disease, allograft rejection, chronic obstructive pulmonary disease; autoimmune diseases such as Graves' disease, rheumatoid arthritis, systemic lupus erythematosus, lupus nephritis, cutaneous lupus, psoriasis; auto-inflammatory diseases including Cryopyrin-Associated Periodic Syndromes (CAPS), TNF Receptor Associated Periodic Syndrome (TRAPS), Familial Mediterranean Fever (FMF), adult onset stills, systemic onset juvenile idiopathic arthritis, gout, gouty arthritis; metabolic diseases including type 2 diabetes, atherosclerosis, myocardial infarction; destructive bone disorders such as bone resorption disease, osteoarthritis, osteoporosis, multiple myeloma-related bone disorder; proliferative disorders such as acute myelogenous leukemia, chronic myelogenous leukemia; angiogenic disorders such as angiogenic disorders including solid tumors, ocular neovascularization, and infantile haemangiomas; infectious diseases such as sepsis, septic shock, and Shigellosis; neurodegenerative diseases such as Alzheimer's disease, Parkinson's disease, cerebral ischemias or neurodegenerative disease caused by traumatic injury, oncologic and viral diseases such as metastatic melanoma, Kaposi's sarcoma, multiple myeloma, and HIV infection and CMV retinitis, AIDS, respectively.

More particularly, the specific conditions or diseases that may be treated with the inventive compounds include, without limitation, pancreatitis (acute or chronic), asthma, allergies, adult respiratory distress syndrome, chronic obstructive pulmonary disease, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic 3-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, keloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, cerebral ischemias or neurodegenerative disease caused by traumatic injury; angiogenic disorders including solid tumors, ocular neovascularization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hypoxia, vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris. Included in this embodiment are methods of treatment in which the condition is selected from lupus including lupus nephritis and systemic lupus erythematosus (SLE), Crohn's disease, ulcerative colitis, allograft rejection, rheumatoid arthritis, psoriasis, ankylosing spondylitis, psoriatic arthritis, and pemphigus vulgaris. Also included are methods of treatment in which the condition is selected from ischemia reperfusion injury, including cerebral ischemia reperfusions injury arising from stroke and cardiac ischemia reperfusion injury arising from myocardial infarction. Another method of treatment is one in which the condition is multiple myeloma.

In one embodiment, the compounds of Formula (I), Formula (II), Formula (III), and Formula (IV) are useful as prodrugs of Compound A, in treating cancer, including Waldenstrom's Macroglobulinemia (WM), diffuse large B cell lymphoma (DLBCL), chronic lymphocytic leukemia (CLL), cutaneous diffuse large B cell lymphoma, and primary CNS lymphoma.

In addition, the TLR7, TLR8, or TLR9 inhibitors of the present invention inhibit the expression of inducible pro-inflammatory proteins such as prostaglandin endoperoxide synthase-2 (PGHS-2), also referred to as cyclooxygenase-2 (COX-2), IL-1, IL-6, IL-18, chemokines. Accordingly, additional TLR7/8/9 associated conditions include edema, analgesia, fever and pain, such as neuromuscular pain, headache, pain caused by cancer, dental pain and arthritis pain. The inventive compounds also may be used to treat veterinary viral infections, such as lentivirus infections, including, but not limited to equine infectious anemia virus; or retrovirus infections, including feline immunodeficiency virus, bovine immunodeficiency virus, and canine immunodeficiency virus.

The present invention thus provides methods for treating such conditions, comprising administering to a subject in need thereof a therapeutically-effective amount of at least one compound of Formula (I), Formula (II), Formula (III), and Formula (IV) or a salt thereof. “Therapeutically effective amount” is intended to include an amount of a compound of the present invention that is effective when administered alone or in combination to inhibit autoimmune disease or chronic inflammatory disease.

The methods of treating TLR7, TLR8, or TLR9 associated conditions may comprise administering at least one compound of Formula (I), Formula (II), Formula (III), and Formula (IV) alone or in combination with each other and/or other suitable therapeutic agents useful in treating such conditions. Accordingly, “therapeutically effective amount” is also intended to include an amount of the combination of compounds claimed that is effective to inhibit TLR7, TLR8, or TLR9 and/or treat diseases associated with TLR7, TLR8, or TLR9.

Exemplary of such other therapeutic agents include corticosteroids, rolipram, calphostin, cytokine-suppressive anti-inflammatory drugs (CSAIDs), Interleukin-10, glucocorticoids, salicylates, nitric oxide, and other immunosuppressants; nuclear translocation inhibitors, such as deoxyspergualin (DSG); non-steroidal anti-inflammatory drugs (NSAIDs) such as ibuprofen, celecoxib and rofecoxib; steroids such as prednisone or dexamethasone; antiviral agents such as abacavir; antiproliferative agents such as methotrexate, leflunomide, FK506 (tacrolimus, PROGRAF®); anti-malarials such as hydroxychloroquine; cytotoxic drugs such as azathiprine and cyclophosphamide; TNF-α inhibitors such as tenidap, anti-TNF antibodies or soluble TNF receptor, and rapamycin (sirolimus or RAPAMUNE®) or derivatives thereof.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds. The present invention also provides pharmaceutical compositions capable of treating TLR7/8/9 receptor-associated conditions, including IL-1 family receptor-mediated diseases as described above.

The inventive compositions may contain other therapeutic agents as described above and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

Accordingly, the present invention further includes compositions comprising at least one compound of Formula (I), Formula (II), Formula (III), and Formula (IV); and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include without limitation the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th Edition (1985), which is incorporated herein by reference in its entirety.

Compounds in accordance with Formula (I), Formula (II), Formula (III), and/or Formula (IV) can be administered by any means suitable for the condition to be treated, which can depend on the need for site-specific treatment or quantity of Compound A to be delivered.

Also embraced within this invention is a class of pharmaceutical compositions comprising at least one compound of Formula (I), Formula (II), Formula (III), and Formula (IV); and one or more non-toxic, pharmaceutically-acceptable carriers and/or diluents and/or adjuvants (collectively referred to herein as “carrier” materials) and, if desired, other active ingredients. The at least one compound of Formula (I), Formula (II), Formula (III), and Formula (IV) may be administered by any suitable route, preferably in the form of a pharmaceutical composition adapted to such a route, and in a dose effective for the treatment intended. The compounds and compositions of the present invention may, for example, be administered orally, mucosally, or parenterally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I), Formula (II), Formula (III), and Formula (IV) with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I), Formula (II), Formula (III), and Formula (IV) with at least one inert solid diluent, such as, for example, calcium carbonate; calcium phosphate; and kaolin.

Soft gelatin capsules can, for example, be prepared by mixing at least one compound of Formula (I), Formula (II), Formula (III), and Formula (IV) with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I), Formula (II), Formula (III), and Formula (IV) with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I), Formula (II), Formula (III), and Formula (IV) in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I), Formula (II), Formula (III), and Formula (IV) with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I), Formula (II), Formula (III), and Formula (IV) thereof can, for example, be prepared as an oil-in-water emulsion. The oily phase of the emulsions comprising at least one compound of Formula (I), Formula (II), Formula (III), and Formula (IV) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The at least one compound of Formula (I), Formula (II), Formula (III), and Formula (IV) can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride solution, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I), Formula (II), Formula (III), and Formula (IV) in an oily phase, such as, for example, a mixture of soybean oil and lecithin; 2) combining the Formula (I), Formula (II), Formula (III), and Formula (IV) containing the oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this invention depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I), Formula (II), Formula (III), and Formula (IV) and optionally an additional agent selected from any pharmaceutically acceptable carrier, adjuvant, and vehicle. Alternate compositions of this invention comprise at least one compound of Formula (I), Formula (II), Formula (III), and Formula (IV) described herein, or salts thereof, and a pharmaceutically acceptable carrier, adjuvant, or vehicle.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt form thereof; and (c) a package insert stating that the pharmaceutical composition can be used for the treatment of an inflammatory disorder and/or an autoimmune disease (as defined previously). In another embodiment, the package insert states that the pharmaceutical composition can be used in combination (as defined previously) with a second therapeutic agent to treat an inflammatory disorder and/or an autoimmune disease. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. That recites information relating to the pharmaceutical composition located within the first container. The information recited will usually be determined by the regulatory agency governing the area in which the article of manufacture is to be sold (e.g., the United States Food and Drug Administration). In one embodiment, the package insert specifically recites the indications for which the pharmaceutical composition has been approved. The package insert may be made of any material on which a person can read information contained therein or thereon. For example, the package insert is a printable material (e.g., paper, plastic, cardboard, foil, adhesive-backed paper or plastic, etc.) on which the desired information has been formed (e.g., printed or applied).

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety by reference.

The compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Third Edition, Wiley and Sons, 1999).

### EXAMPLES

Preparation of compounds of Formula (I), and intermediates used in the preparation of compounds of Formula (I), can be prepared using procedures shown in the following Examples and related procedures. The methods and conditions used in these examples, and the actual compounds prepared in these Examples, are not meant to be limiting, but are meant to demonstrate how the compounds of Formula (I) can be prepared. Starting materials and reagents used in these examples, when not prepared by a procedure described herein, are generally either commercially available, or are reported in the chemical literature, or may be prepared by using procedures described in the chemical literature.

### Abbreviations

Ac acetyl
ACN acetonitrile
AcOH acetic acid
anhyd. anhydrous
aq. aqueous
Bn benzyl
Boc tert-butoxycarbonyl

### CV Column Volumes

DBU 1,8-diazabicyclo[5.4.0]undec-7-ene
DCC 1,3-dicyclohexylcarbodiimide
DCE dichloroethane
DCM dichloromethane
DIPEA diisopropylethylamine
DMAP dimethylaminopyridine
DMF dimethylformamide
DMSO dimethylsulfoxide
EDC 1-(3-dimethylaminopropyl)-3-ethylcarbodiimide hydrochloride
Et₃N triethyl amine
EtO₂ diethyl ether
EtOAc ethyl acetate
Et ethyl
EtOH ethanol
H or H₂ hydrogen
h, hr or hrs hour(s)
hex hexane
i iso
IPA isopropyl alcohol
HOAc acetic acid
HCl hydrochloric acid
HPLC high pressure liquid chromatography
LC liquid chromatography
LiHMDS lithium bis(trimethylsilyl)amide
M molar
mM millimolar
Me methyl
MeOH methanol
MHz megahertz
min. minute(s)
mins minute(s)
M⁺¹ (M+H)⁺
MS mass spectrometry
n or N normal
NH₄OAc ammonium acetate
nm nanometer
nM nanomolar
Pd/C palladium on carbon
Ph phenyl
Pr propyl
PSI pounds per square inch
RT retention time
sat. saturated
SFC supercritical fluid chromatography
TBAI tetrabutylammonium iodide
TFA trifluoroacetic acid
THE tetrahydrofuran
Analytical Purity of the compounds was determined by using the following methods:

Analytical HPLC Method A: Column: SunFire C18 (150×4.6 mm) 3.5 micron; buffer: 0.05% CF₃CO₂H in H₂O; mobile phase A=buffer: CH₃CN [95:5]; mobile phase B: CH₃CN: buffer [95:5]; 10% B to 100% B; run time: 23 min; flow rate: 1.0 mL/min.

Analytical HPLC Method B: Column: XBridge Phenyl C18 (150×4.6 mm) 3.5 micron; buffer: 0.05% CF₃CO₂H in H₂O; mobile phase A=buffer: CH₃CN [95:5]; mobile phase B: CH₃CN: buffer [95:5]; 10% B to 100% B; run time: 23 min; flow rate: 1.0 mL/min.

Analytical HPLC Method C: Column: Kinetex EVO C18 (4.6×100) mm, 2.6 micron; buffer: 0.05% CF₃CO₂H in H₂O; mobile phase A=buffer: CH₃CN [95:5]; mobile phase B: CH₃CN: buffer [95:5]; 2% B to 100% B; run time: 12.5 min; flow rate: 1.0 mL/min.

Analytical HPLC Method D: Column: XBridge Phenyl C18 (150×4.6 mm) 3.5 micron; buffer: 0.05% CF₃CO₂H in H₂O; mobile phase A=buffer: CH₃CN [95:5]; mobile phase B: CH₃CN: buffer [95:5]; 0% B to 100% B; run time: 12.5 min; flow rate: 1.0 mL/min.

Analytical HPLC Method E: Column: Kinetex EVO C18 (4.6×100) mm, 2.6 micron; buffer: 0.05% CF₃CO₂H in H₂O; mobile phase A=buffer: CH₃CN [95:5]; mobile phase B: CH₃CN: buffer [95:5]; 5% B to 100% B; run time: 35 min; flow rate: 1.0 mL/min.

Analytical HPLC Method F: Column: Kinetex Biphenyl C18 (4.6×100) mm, 2.6 micron; buffer: 0.05% CF₃CO₂H in H₂O; mobile phase A=buffer: CH₃CN [95:5]; mobile phase B: CH₃CN: buffer [95:5]; 5% B to 100% B; run time: 35 min; flow rate: 1.0 mL/min.

### Example 1

### (S)-Piperidin-2-ylmethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

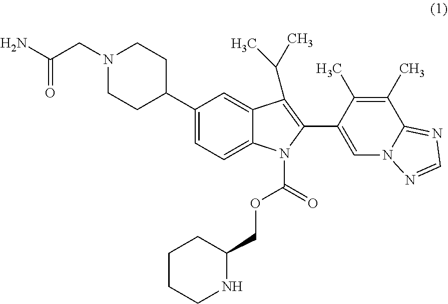 [SMILES: Cc1c(-c2c(C(C)C)c3cc(C4CCN(CC(N)=O)CC4)ccc3n2C(=O)OC[C@@H]2CCCCN2)cn2ncnc2c1C]

### Intermediate 1A: tert-Butyl (S)-2-(hydroxymethyl)piperidine-1-carboxylate

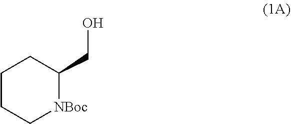 [SMILES: CC(C)(C)OC(=O)N1CCCC[C@H]1CO]

(S)-1-Boc-piperidine-2-carboxylic acid (4 g, 17.45 mmol) was dissolved in THF (40 mL) and cooled to 0° C. Borane dimethyl sulfide complex (2.70 mL, 30.5 mmol) was then added drop wise at 0° C. The reaction mixture was stirred for 1 h. The reaction mixture was then warmed to 25° C. and stirred for an additional 12 h. The reaction mixture were cooled to 0° C. Saturated aqueous NaHCO₃ solution (40 mL) was added slowly to quench the excess reagent. Water (20 mL) was added to dissolve the precipitated salts. The crude reaction contents were then extracted with CH₂Cl₂ (4×50 mL), and the combined organic extracts were washed with saturated aqueous NaHCO₃ and H₂O, dried (MgSO₄), filtered, and concentrated to afford the title compound (3.7 g, 16.33 mmol, 94% yield). ¹H NMR (400 MHz, DMSO-d₆) δ 4.63 (t, J=5.5 Hz, 1H), 4.03 (q, J=6.5 Hz, 1H), 3.85-3.77 (m, 1H), 3.50 (ddd, J=10.5, 8.5, 6.0 Hz, 1H), 3.43-3.34 (m, 1H), 2.72 (t, J=13.1 Hz, 1H), 1.81-1.72 (m, 1H), 1.60-1.52 (m, 1H), 1.52-1.44 (m, 2H), 1.44-1.33 (m, 10H), 1.30-1.19 (m, 1H).

### Intermediate 1B: tert-Butyl (S)-2-(((1H-imidazole-1-carbonyl)oxy)methyl)piperidine-1-carboxylate

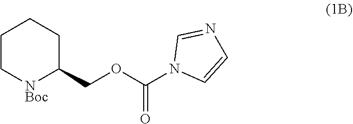 [SMILES: CC(C)(C)OC(=O)N1CCCC[C@H]1COC(=O)n1ccnc1]

To a solution of tert-butyl (S)-2-(hydroxymethyl)piperidine-1-carboxylate (3.04 g, 14.12 mmol) in CH₃CN (40 mL), were added DIPEA (7.40 mL, 42.4 mmol) and 1,1′-carbonyldiimidazole (4.58 g, 28.2 mmol). The reaction mixture was stirred at room temperature for 8 h. The reaction mixture was concentrated to remove the solvent. The residue was partitioned between EtOAc and H₂O. The organic layer was separated and washed with brine, dried over anhydrous Na₂SO₄, filtered and concentrated to afford a colorless gum. The crude material was purified by CombiFlash (silica gel 60-120 mesh; 50% EtOAc in hexane as eluent) to afford the title compound (4.2 g, 12.90 mmol, 91% yield) as a white solid. ¹H NMR (400 MHz, CDCl₃) δ 8.13 (s, 1H), 7.42 (t, J=1.5 Hz, 1H), 7.05 (s, 1H), 4.76-4.57 (m, 2H), 4.38 (dd, J=10.8, 5.0 Hz, 1H), 4.15-4.01 (m, 1H), 2.94-2.81 (m, 1H), 1.77-1.64 (m, 4H), 1.55-1.41 (m, 2H), 1.36 (s, 9H). LC-MS (ES): m/z=308.3 [M+H]⁺.

### Intermediate 1C: (S)-(1-(tert-Butoxycarbonyl)piperidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

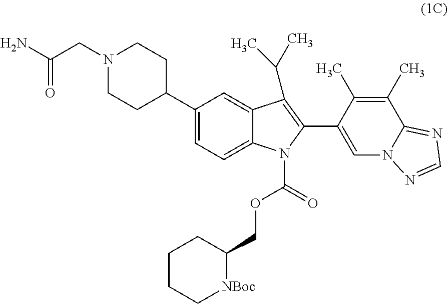 [SMILES: Cc1c(-c2c(C(C)C)c3cc(C4CCN(CC(N)=O)CC4)ccc3n2C(=O)OC[C@@H]2CCCCN2C(=O)OC(C)(C)C)cn2ncnc2c1C]

To a solution of 2-(4-(2-(7,8-dimethyl-[1,2,4]triazolo[1,5-a]pyridin-6-yl)-3-isopropyl-1H-indol-5-yl)piperidin-1-yl)acetamide (1 g, 2.249 mmol) in DMF (10 mL), were added DBU (0.509 mL, 3.37 mmol) and tert-butyl (S)-2-(((1H-imidazole-1-carbonyl)oxy)methyl)piperidine-1-carboxylate (2.088 g, 6.75 mmol). The reaction mixture was stirred at room temperature for 16 h. Additional amounts of tert-butyl (S)-2-(((1H-imidazole-1-carbonyl)oxy)methyl)piperidine-1-carboxylate (1.0 equiv.) and DBU (1.0 equiv.) were added. The reaction mixture was stirred for an additional 8 h. The reaction mixture was partitioned between EtOAc and H₂O. The organic layer was separated, washed with brine, dried over anhydrous Na₂SO₄, filtered and concentrated to yield a colorless gum. The crude material was purified by CombiFlash (silica gel 60-120 mesh; 50% EtOAc in hexane as eluent) to afford the title compound (650 mg, 0.929 mmol, 41.3% yield) as a white solid. LC-MS (ES): m/z=686.7 [M+H]⁺.

### Example 1

To a stirred solution of (S)-(1-(tert-butoxycarbonyl)piperidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (650 mg, 0.948 mmol) in CH₂Cl₂ (5 mL), was added CF₃CO₂H (1 mL, 12.98 mmol) at 0° C. The reaction mixture was stirred at 0° C. for 10 min, and then stirred for 30 min at room temperature. The reaction mixture was concentrated to remove the solvent at 30° C. The crude product was purified using RP-HPLC (column: SunFire C18 (250×21.2 mm), 5 micron; mobile phase A: 0.1% CF₃CO₂H in H₂O; mobile phase B: CH₃CN; flow rate: 20 mL/min; gradient (time (min)/% B): 0/10, 2/10, 10/25). The fraction was concentrated using high vacuum at 30° C. The residue was dissolved in a mixture of CH₃CN and H₂O, frozen and lyophilized for 12 h to afford the title product (650 mg, 0.791 mmol, 83% yield) as a white solid. ¹H NMR (400 MHz, DMSO-d₆) δ 9.69 (br. s., 1H), 8.97-8.89 (m, 1H), 8.72 (d, J=3.5 Hz, 1H), 8.59-8.45 (m, 1H), 8.21-8.08 (m, 1H), 8.05 (s, 1H), 7.71-7.75 (m, 2H), 7.35-7.27 (m, 1H), 4.43-4.12 (m, 2H), 3.98 (s, 2H), 3.61 (d, J=11.5 Hz, 2H), 3.30-3.15 (m, 3H), 3.08 (br. s., 1H), 2.98 (t, J=12.0 Hz, 1H), 2.76 (dt, J=14.2, 7.2 Hz, 2H), 2.63-2.55 (m, 3H), 2.23-1.97 (m, 7H), 1.72-1.53 (m, 2H), 1.40-1.20 (m, 8H), 1.19-1.06 (m, 1H), 0.88-0.74 (m, 1H). LC-MS (ES): m/z=586.4 [M+H]⁺; HPLC RT and purity: method A=7.597 min and 99.84% and method B=7.866 min and 99.85%.

### Example 2

### (S)-(1-(((Phosphonooxy)methoxy)carbonyl)piperidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

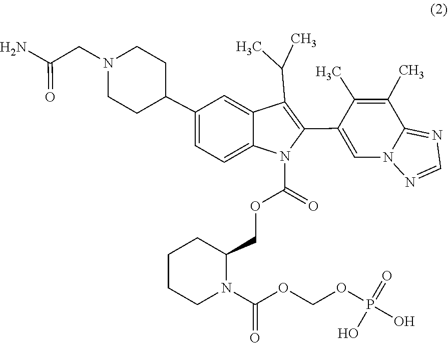 [SMILES: Cc1c(-c2c(C(C)C)c3cc(C4CCN(CC(N)=O)CC4)ccc3n2C(=O)OC[C@@H]2CCCCN2C(=O)OCOP(=O)(O)O)cn2ncnc2c1C]

### Intermediate 2A: (S)-(1-((((bis(Benzyloxy)phosphoryl)oxy)methoxy)carbonyl)piperidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

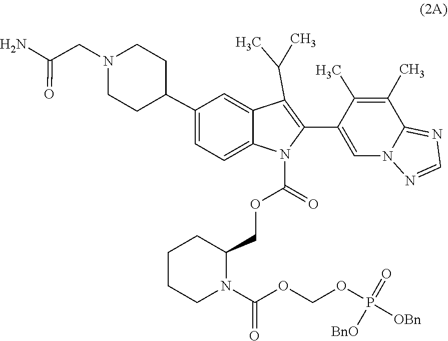 [SMILES: Cc1c(-c2c(C(C)C)c3cc(C4CCN(CC(N)=O)CC4)ccc3n2C(=O)OC[C@@H]2CCCCN2C(=O)OCOP(=O)(OCc2ccccc2)OCc2ccccc2)cn2ncnc2c1C]

To a solution of (S)-piperidin-2-ylmethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (150 mg, 0.184 mmol) in THE (5 mL), was added DIPEA (0.161 mL, 0.922 mmol). The reaction mixture was cooled to 0° C. and then ((bis(benzyloxy)phosphoryl) oxy)methyl chloroformate (137 mg, 0.369 mmol) was added. The reaction mixture was brought to room temperature and stirred for 2 h. The reaction mixture was partitioned between EtOAc and H₂O. The organic layer was separated, washed with brine, dried over anhydrous Na₂SO₄, filtered and concentrated to yield a colorless gum. The crude material was purified using RP-HPLC (column: YMC TRIAT (250×20 mm), 5 micron; mobile phase A: 10 mM NH₄OAc in H₂O; mobile phase B: CH₃CN; flow rate: 20 mL/min; gradient (time (min)/% B): 0/60, 15/85). The fraction was concentrated using high vacuum at 30° C. The residue was dissolved in a mixture of CH₃CN and H₂O, frozen and lyophilized for 12 h to afford the title compound (55 mg, 0.059 mmol, 31.8% yield) as a white solid. ¹H NMR (400 MHz, DMSO-d₆) δ 8.75-8.60 (m, 1H), 8.43 (s, 1H), 8.05-7.99 (m, 1H), 7.64 (s, 1H), 7.43-7.21 (m, 13H), 5.61-5.24 (m, 2H), 5.07-4.93 (m, 4H), 4.44-3.74 (m, 4H), 2.99-2.59 (m, 10H), 2.20 (t, J=9.8 Hz, 2H), 2.11-1.99 (m, 3H), 1.90-1.72 (m, 4H), 1.42-0.99 (m, 12H). LC-MS (ES): m/z=920.4 [M+H]⁺.

### Example 2

A stirred solution of (S)-(1-((((bis(benzyloxy)phosphoryl)oxy)methoxy)carbonyl) piperidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (55 mg, 0.060 mmol) in MeOH (4 mL) was purged with nitrogen. Pd/C (31.8 mg, 0.030 mmol) was then added. The reaction mixture was stirred under hydrogen atmosphere using a bladder at room temperature for 2 h. The reaction mixture was filtered through a Celite bed and washed with MeOH. The organic layer was concentrated under high vacuum at 30° C. to yield a colorless gum. The crude product was purified using RP HPLC (column: SunFire C18 (250×21.2 mm), 5 micron; mobile phase A: 0.1% CF₃CO₂H in H₂O; mobile phase B: CH₃CN; flow rate: 20 mL/min; gradient (time (min)/% B): 0/20, 2/20, 9/35). The fraction was concentrated using high vacuum at 30° C. The residue was dissolved in a mixture of CH₃CN and H₂O, frozen and lyophilized for 12 h to afford the title compound (35 mg, 0.040 mmol, 67.2% yield) as a mixture of isomers (white solid). ¹H NMR (400 MHz, MeOD) δ 8.55-8.49 (m, 1H), 8.41 (d, J=4.00 Hz, 1H), 8.11-8.03 (m, 1H), 7.61 (s, 1H), 7.26-7.24 (m, 1H), 5.33-5.27 (m, 1H), 5.00-4.85 (m, 1H), 4.50-4.10 (m, 3H), 3.90 (s, 3H), 3.69-3.65 (m, 2H), 3.5-3.3 (m, 1H), 3.20-3.10 (m, 2H), 3.00-2.90 (m, 1H), 2.85-2.76 (m, 1H), 2.56-2.54 (m, 3H), 2.10-2.06 (m, 7H), 1.60-1.15 (m, 12H). LC-MS (ES): m/z=740.4 [M+H]⁺; HPLC RT and purity: method A=5.188-5.198 min and 98.24%; method B=4.932-4.994 min and 98.67%.

### Example 3

### (S)-Pyrrolidin-2-ylmethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

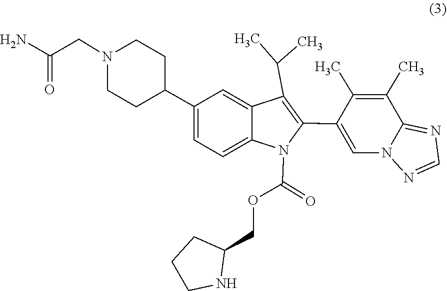 [SMILES: Cc1c(-c2c(C(C)C)c3cc(C4CCN(CC(N)=O)CC4)ccc3n2C(=O)OC[C@@H]2CCCN2)cn2ncnc2c1C]

### Intermediate 3A: (S)-(1-(tert-Butoxycarbonyl)pyrrolidin-2-yl)methyl 1H-imidazole-1-carboxylate

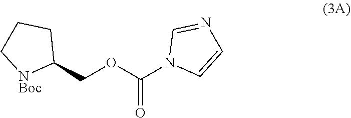 [SMILES: CC(C)(C)OC(=O)N1CCC[C@H]1COC(=O)n1ccnc1]

To a solution of tert-butyl (S)-2-(hydroxymethyl)pyrrolidine-1-carboxylate (5 g, 24.84 mmol) in CH₃CN (50 mL), were added DIPEA (13.02 mL, 74.5 mmol) and 1,1′-carbonyldiimidazole (8.06 g, 49.7 mmol). The reaction mixture was stirred at room temperature for 8 h. The reaction mixture was concentrated to remove the solvent. The residue was partitioned between EtOAc and H₂O. The organic layer was separated and washed with brine, dried over anhydrous Na₂SO₄, filtered and concentrated to yield a colorless gum. The crude product was purified by CombiFlash (silica gel 60-120 mesh; 40% EtOAc in hexane as an eluent) to afford the title compound (6.2 g, 19.94 mmol, 80% yield) as a colorless oil. ¹H NMR (300 MHz, CDCl₃) δ 8.14 (s, 1H), 7.43 (s, 1H), 7.08 (s, 1H), 4.51-4.34 (m, 2H), 4.25-4.12 (m, 1H), 3.47 (br. s., 1H), 3.38 (br. s., 1H), 2.13-1.99 (m, 1H), 1.98-1.78 (m, 3H), 1.45 (s, 9H). LC-MS (ES): m/z=296.4 [M+H]⁺;

### Intermediate 3B: (S)-(1-(tert-Butoxycarbonyl)pyrrolidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

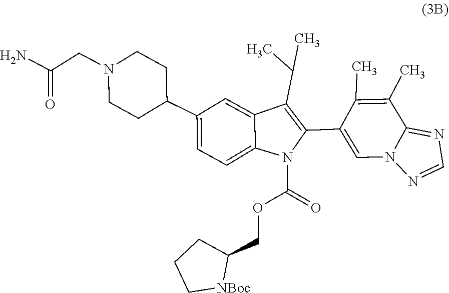 [SMILES: Cc1c(-c2c(C(C)C)c3cc(C4CCN(CC(N)=O)CC4)ccc3n2C(=O)OC[C@@H]2CCCN2C(=O)OC(C)(C)C)cn2ncnc2c1C]

To a solution of 2-(4-(2-(7,8-dimethyl-[1,2,4]triazolo[1,5-a]pyridin-6-yl)-3-isopropyl-1H-indol-5-yl)piperidin-1-yl)acetamide (527 mg, 1.185 mmol) in DMF (8 mL), were added DBU (0.268 mL, 1.778 mmol) and (S)-(1-(tert-butoxycarbonyl)pyrrolidin-2-yl)methyl 1H-imidazole-1-carboxylate (1050 mg, 3.56 mmol). The reaction mixture was stirred at room temperature for 8 h. The reaction mixture was partitioned between EtOAc and H₂O. The separated organic layer was washed with brine, dried over anhydrous Na₂SO₄, filtered and concentrated to yield a colorless gum. The crude material was purified by CombiFlash (silica gel 60-120 mesh; 35 to 40% EtOAc in hexane as eluent) to afford the title compound (350 mg, 0.495 mmol, 41.8% yield) as a white solid. ¹H NMR (300 MHz, DMSO-d₆) δ 8.81 (br. s., 1H), 8.46 (d, J=2.5 Hz, 1H), 8.18-8.06 (m, 1H), 7.67 (s, 1H), 7.31 (d, J=9.4 Hz, 2H), 7.16 (br. s., 1H), 4.16-3.96 (m, 1H), 3.92 (br. s., 1H), 3.07 (d, J=5.9 Hz, 2H), 3.00-2.86 (m, 4H), 2.81-2.62 (m, 3H), 2.57 (s, 3H), 2.31-2.13 (m, 2H), 2.08 (s, 3H), 1.94-1.85 (m, 2H), 1.81 (br. s., 3H), 1.61 (d, J=6.8 Hz, 1H), 1.51 (d, J=7.4 Hz, 2H), 1.38-1.18 (m, 15H). LC-MS (ES): m/z=672.7 [M+H]⁺.

### Example 3

To a stirred solution of (S)-(1-(tert-butoxycarbonyl)pyrrolidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (450 mg, 0.670 mmol) in CH₂Cl₂ (5 mL) at 0° C., CF₃CO₂H (1 mL, 12.98 mmol) was added. The reaction mixture was stirred at 0° C. for 10 min, brought to room temperature, and stirred for 30 min. The reaction mixture was concentrated to remove the solvent at 30° C. The crude product was purified using RP HPLC (column: SunFire C18 (150×19 mm), 5 micron; mobile phase A: 0.1% CF₃CO₂H in H₂O; mobile phase B: CH₃CN; flow rate: 17 mL/min; gradient (time (min)/% B): 0/10, 10/30). The fraction was concentrated using high vacuum at 30° C. The residue was dissolved in a mixture of CH₃CN and H₂O, frozen and lyophilized for 12 h to afford the title compound (320 mg, 0.396 mmol, 59.1% yield) as a white solid. ¹H NMR (300 MHz, DMSO-d₆) δ 9.64 (br. s., 1H), 9.38 (br. s., 1H), 8.98-8.85 (m, 1H), 8.73 (d, J=3.0 Hz, 1H), 8.48 (d, J=2.0 Hz, 1H), 8.10-8.15 (d, J=8.6 Hz, 1H), 8.03 (s, 1H), 7.70 (s, 1H), 7.74 (s, 1H), 7.28 (d, J=8.6 Hz, 1H), 4.51-4.31 (m, 3H), 3.98 (br. s., 2H), 3.59-3.18 (m, 5H), 2.98 (t, J=11.6 Hz, 1H), 2.82-2.68 (m, 1H), 2.58 (s, 3H), 2.24-1.96 (m, 7H), 1.95-1.80 (m, 1H), 1.71 (d, J=4.6 Hz, 2H), 1.54-1.21 (m, 8H); LC-MS (ES): m/z=572.4 [M+H]⁺; HPLC RT and purity: Method A=7.167 min and 99.54% and method B=7.912 min and 99.59%.

### Example 4

### (S)-(1-(((Phosphonooxy)methoxy)carbonyl)pyrrolidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

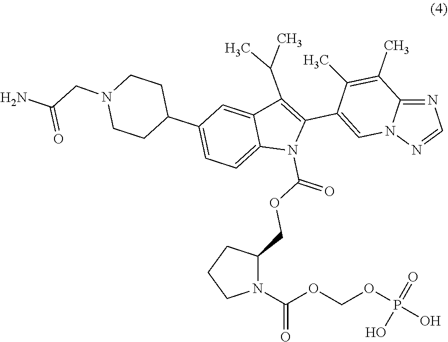 [SMILES: Cc1c(-c2c(C(C)C)c3cc(C4CCN(CC(N)=O)CC4)ccc3n2C(=O)OC[C@@H]2CCCN2C(=O)OCOP(=O)(O)O)cn2ncnc2c1C]

### Intermediate 4A: (S)-(1-((((bis(Benzyloxy)phosphoryl)oxy)methoxy)carbonyl)pyrrolidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

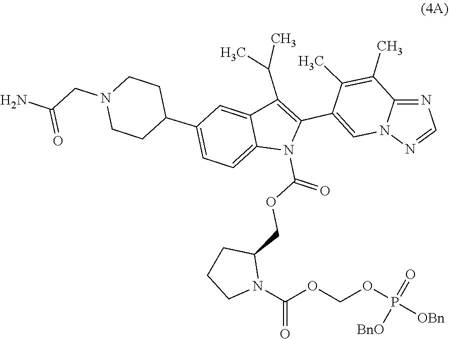 [SMILES: Cc1c(-c2c(C(C)C)c3cc(C4CCN(CC(N)=O)CC4)ccc3n2C(=O)OC[C@@H]2CCCN2C(=O)OCOP(=O)(OCc2ccccc2)OCc2ccccc2)cn2ncnc2c1C]

To a solution of (S)-pyrrolidin-2-ylmethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate ditrifluoroacetate (125 mg, 0.156 mmol) in THE (5 mL), were sequentially added Et₃N (0.109 mL, 0.781 mmol) and ((bis(benzyloxy)phosphoryl)oxy)methyl chloroformate (116 mg, 0.313 mmol). The reaction mixture was brought to room temperature and stirred for 2 h. The reaction mixture was partitioned between EtOAc and H₂O. The organic layer was separated, washed with brine, dried over anhydrous Na₂SO₄, filtered and concentrated to yield a colorless gum. The crude material was purified using RP HPLC (column: X-Bridge phenyl (250×19 mm), 5 micron; mobile phase A: 10 mM NH₄OAc in H₂O; mobile phase B: CH₃CN; flow rate: 20 mL/min; gradient (time (min)/% B): 0/35, 2/35, 14/65). The fraction was concentrated using high vacuum at 30° C. The residue was dissolved in a mixture of CH₃CN and H₂O, frozen and lyophilized for 12 h to afford the title product (45 mg, 0.045 mmol, 28.6% yield) as a white solid. ¹H NMR (300 MHz, DMSO-d₆) δ 8.83-8.72 (m, 1H), 8.49-8.41 (m, 1H), 8.12-8.00 (m, 1H), 7.66 (s, 1H), 7.57 (d, J=5.0 Hz, 1H), 7.41-7.24 (m, 11H), 7.16 (br. s., 1H), 5.63-5.43 (m, 2H), 5.02 (d, J=7.4 Hz, 4H), 4.21-3.80 (m, 2H), 3.20-3.01 (m, 2H), 3.01-2.86 (m, 4H), 2.79-2.62 (m, 3H), 2.54 (br. s., 3H), 2.30-2.19 (m, 3H), 2.15 (br. s., 1H), 2.00-1.81 (m, 4H), 1.52-1.31 (m, 2H), 1.35-1.19 (m, 8H), 1.01 (d, J=6.6 Hz, 1H). LC-MS (ES): m/z=906.8 [M+H]⁺

### Example 4

A solution of (S)-(1-((((bis(benzyloxy)phosphoryl)oxy)methoxy)carbonyl) pyrrolidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (20 mg, 0.022 mmol) in MeOH (1 mL) was purged with nitrogen. Subsequently, Pd/C (11.75 mg, 0.011 mmol) was added. The reaction mixture was stirred under hydrogen atmosphere using a bladder at room temperature for 2 h. The reaction mixture was filtered through a Celite bed and washed with MeOH. The organic layer was concentrated under high vacuum at 30° C. to yield a colorless gum. The crude product was purified using RP HPLC (column: SunFire C18 (150×21.2 mm), 5 micron; mobile phase A: 0.1% CF₃CO₂H in H₂O; mobile phase B: CH₃CN; flow rate: 20 mL/min; gradient (time (min)/% B): 0/10, 2/10, 15/40). The fraction was concentrated using high vacuum at 30° C. The residue was dissolved in a mixture of CH₃CN and H₂O, frozen and lyophilized for 12 h to afford the title compound (20 mg, 0.022 mmol) as a white solid. ¹H NMR (400 MHz, MeOH-d₄) δ 8.55-8.52 (m, 1H), 8.43-8.32 (m, 1H), 8.13 (t, J=8.2 Hz, 1H), 7.62 (br. s., 1H), 7.26 (d, J=6.8 Hz, 1H), 5.42 (br. s., 1H), 5.36 (br. s., 1H), 4.38-4.27 (m, 1H), 4.12-3.99 (m, 2H), 3.91-3.85 (m, 2H), 3.68-3.66 (m, 4H), 2.97-3.30 (m, 3H), 2.75 (dd, J=13.9, 7.3 Hz, 1H), 2.55 (br. s., 3H), 2.18-1.96 (m, 7H), 1.71-1.54 (m, 3H), 1.32-1.21 (m, 7H). LC-MS (ES): m/z=727.2 [M+H]⁺; HPLC RT and purity: method A=4.559 min and 99.40% and method B=4.425 min and 99.46%.

### Example 5

### 2-(4-(2-(7,8-Dimethyl-[1,2,4]triazolo[1,5-a]pyridin-6-yl)-3-isopropyl-1-(methylglycyl)-1H-indol-5-yl)piperidin-1-yl)acetamide ditrifluoroacetate

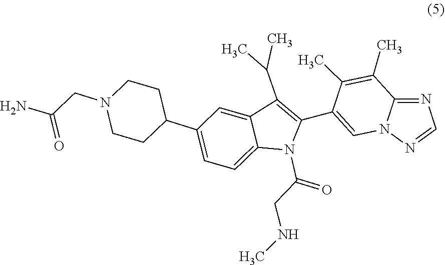 [SMILES: CNCC(=O)n1c(-c2cn3ncnc3c(C)c2C)c(C(C)C)c2cc(C3CCN(CC(N)=O)CC3)ccc21]

### Intermediate 5A: Benzyl 4-(2-(7,8-dimethyl-[1,2,4]triazolo[1,5-a]pyridin-6-yl)-3-isopropyl-1H-indol-5-yl)piperidine-1-carboxylate

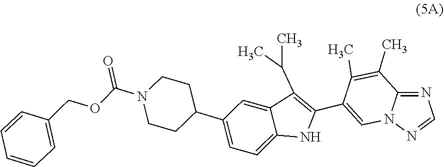 [SMILES: Cc1c(-c2[nH]c3ccc(C4CCN(C(=O)OCc5ccccc5)CC4)cc3c2C(C)C)cn2ncnc2c1C]

Et₃N (0.809 mL, 5.81 mmol) and benzyl chloroformate (1.105 mL, 3.87 mmol) were added to a solution of 6-(3-isopropyl-5-(piperidin-4-yl)-1H-indol-2-yl)-7,8-dimethyl-[1,2,4]triazolo[1,5-a]pyridine (1.5 g, 3.87 mmol) in CH₂Cl₂ (5 mL) at 0° C. The reaction mixture was then stirred at room temperature for 16 h. The resulting suspension was filtered and the residue after solvent evaporation was taken up in EtOAc (50 mL), which was washed with H₂O and dried over anhydrous Na₂SO₄. The solvent was removed to yield the crude product, which was purified by CombiFlash chromatography (60-120 silica gel; 20-60% EtOAc in petroleum ether as eluent) to afford the title compound (0.8 g) as an off-white solid. ¹H NMR (400 MHz, DMSO-d₆) δ 11.01-10.91 (m, 1H), 8.74 (s, 1H), 8.48 (s, 1H), 7.61-7.52 (m, 1H), 7.44-7.24 (m, 6H), 7.08-6.97 (m, 1H), 5.12 (s, 2H), 4.19 (d, J=13.6 Hz, 2H), 3.06-2.77 (m, 4H), 2.63-2.55 (m, 3H), 2.16 (s, 3H), 2.01-1.91 (m, 1H), 1.84 (d, J=11.0 Hz, 1H), 1.78-1.53 (m, 2H), 1.38-1.26 (m, 6H). LC-MS (ES): m/z=522.2 [M+H]⁺.

### Intermediate 5B: Benzyl 4-(1-(N-(tert-butoxycarbonyl)-N-methylglycyl)-2-(7,8-dimethyl-[1,2,4]triazolo[1,5-a]pyridin-6-yl)-3-isopropyl-1H-indol-5-yl)piperidine-1-carboxylate

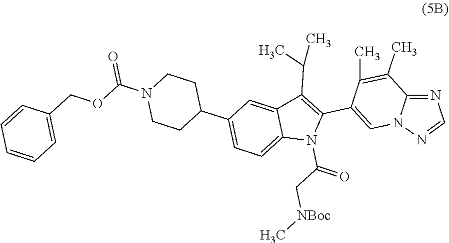 [SMILES: Cc1c(-c2c(C(C)C)c3cc(C4CCN(C(=O)OCc5ccccc5)CC4)ccc3n2C(=O)CN(C)C(=O)OC(C)(C)C)cn2ncnc2c1C]

To a solution of benzyl 4-(2-(7,8-dimethyl-[1,2,4]triazolo[1,5-a]pyridin-6-yl)-3-isopropyl-1H-indol-5-yl)piperidine-1-carboxylate (500 mg, 0.958 mmol) in CHCl₃ (10 mL), were added N-(tert-butoxycarbonyl)-N-methylglycine (544 mg, 2.88 mmol), DCC (297 mg, 1.438 mmol) and DMAP (117 mg, 0.958 mmol). The reaction mixture was stirred at 60° C. for 16 h under N2, filtered through Celite bed and the bed was washed with EtOAc. The filtrate was concentrated under vacuum at 30° C. The crude product was purified using RP HPLC (Column: Xbridge Phenyl (250×4.6 mm) 5 micron; mobile phase A: 10 mM ammonium bicarbonate—pH 9.5 mobile phase B: CH₃CN; flow rate: 1 mL/min; gradient (time (min)/% B): 0/30, 3/60, 15/100, 19/100, 20/30). The fraction was concentrated using high vacuum at 30° C. The residue was dissolved in a mixture of CH₃CN and H₂O, frozen and lyophilized for 12 h to afford the title compound (400 mg, 0.577 mmol, 60.2% yield) as a brownish solid. ¹H NMR (400 MHz, DMSO-d₆) δ 8.87 (d, J=2.0 Hz, 1H), 8.55-8.48 (m, 1H), 8.29-8.15 (m, 1H), 7.69 (s, 1H), 7.45-7.27 (m, 6H), 5.12 (s, 2H), 4.20 (d, J=13.1 Hz, 2H), 4.14-3.88 (m, 2H), 3.08-2.83 (m, 3H), 2.75 (s, 1H), 2.70-2.64 (m, 3H), 2.59 (d, J=8.5 Hz, 3H), 2.57 (s, 3H), 1.85 (d, J=12.5 Hz, 2H), 1.65 (qd, J=12.6, 4.3 Hz, 2H), 1.35-1.20 (m, 15H). LC-MS (ES): m/z=693.4 [M+H]⁺.

### Intermediate 5C: tert-Butyl (2-(2-(7,8-dimethyl-[1,2,4]triazolo[1,5-a]pyridin-6-yl)-3-isopropyl-5-(piperidin-4-yl)-1H-indol-1-yl)-2-oxoethyl)(methyl)carbamate

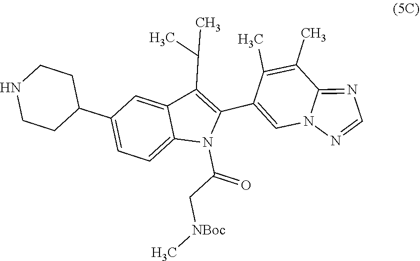 [SMILES: Cc1c(-c2c(C(C)C)c3cc(C4CCNCC4)ccc3n2C(=O)CN(C)C(=O)OC(C)(C)C)cn2ncnc2c1C]

To a solution of benzyl 4-(1-(N-(tert-butoxycarbonyl)-N-methylglycyl)-2-(7,8-dimethyl-[1,2,4]triazolo[1,5-a]pyridin-6-yl)-3-isopropyl-1H-indol-5-yl)piperidine-1-carboxylate (440 mg, 0.635 mmol) in dry EtOAc (5 mL), was added Pd/C (43.9 mg, 0.041 mmol). The mixture was degassed and then flushed with N2 gas. The reaction mixture was stirred for 5 h under H₂ atmosphere, filtered through Celite bed and the bed was washed with EtOAc. The filtrate was concentrated under vacuum at 30° C. to afford the title compound (334 mg, 98%). ¹H NMR (400 MHz, DMSO-d₆) δ 8.87 (s, 1H), 8.50 (d, J=7.5 Hz, 1H), 8.27-8.14 (m, 1H), 7.65 (s, 1H), 7.29 (dd, J=9.3, 3.8 Hz, 1H), 4.14-3.88 (m, 2H), 3.05-3.08 (m, 2H), 2.75 (s, 3H), 2.70-2.58 (m, 7H), 2.14-2.07 (m, 3H), 1.81-1.70 (m, 2H), 1.61 (q, J=12.4 Hz, 2H), 1.37-1.20 (m, 15H; LC-MS (ES): m/z=559.2 [M+H]⁺.

### Intermediate 5D: tert-Butyl (2-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-2-oxoethyl) (methyl)carbamate

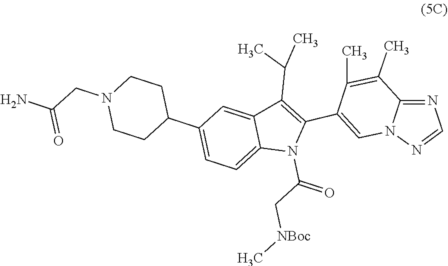 [SMILES: Cc1c(-c2c(C(C)C)c3cc(C4CCN(CC(N)=O)CC4)ccc3n2C(=O)CN(C)C(=O)OC(C)(C)C)cn2ncnc2c1C]

To a solution of tert-butyl (2-(2-(7,8-dimethyl-[1,2,4]triazolo[1,5-a]pyridin-6-yl)-3-isopropyl-5-(piperidin-4-yl)-1H-indol-1-yl)-2-oxoethyl)(methyl)carbamate (300 mg, 0.537 mmol) in DMF (10 mL), were added Et₃N (0.225 mL, 1.611 mmol) and 2-bromoacetamide (96 mg, 0.698 mmol). The reaction mixture was stirred at 0° C. for 1 h. The reaction mixture was diluted with H₂O and extracted with EtOAc. The organic layer was washed with H₂O and brine, dried over anhydrous Na₂SO₄ and concentrated under vacuum at 40° C. to yield the crude product as brownish oil, which was purified by RP HPLC (Column: YMC Trait (150×20) mm 5 micron; mobile phase A: 10 mM NH₄OAc in H₂O; mobile phase B: CH₃CN; flow rate: 20 mL/min; gradient (T/% B): 0/40, 2/40, 12/65) to afford the title compound (100 mg, 0.161 mmol, 29.9% yield) as a white solid. ¹H NMR (400 MHz, DMSO-d₆) δ 8.87 (br. s., 1H), 8.51 (d, J=7.5 Hz, 1H), 8.27-8.16 (m, 1H), 7.69 (s, 1H), 7.34 (br. s., 1H), 7.26 (br. s., 1H), 7.14 (br. s., 1H), 4.14-3.91 (m, 2H), 2.95 (m, 4H), 2.75-2.58 (m, 8H), 2.26-2.16 (m, 2H), 2.13-2.05 (s, 3H), 1.85-1.59 (m, 4H), 1.34-1.10 (m, 15H). LC-MS (ES): m/z=616.4 [M+H]⁺.

### Example 5

To a solution of tert-butyl (2-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-2-oxoethyl) (methyl)carbamate (75 mg, 0.122 mmol) in dry CH₂Cl₂ (2 mL) at 0° C. under nitrogen atmosphere, was added CF₃CO₂H (0.3 mL, 3.89 mmol). After being stirred for 30 min, the reaction mixture was concentrated under vacuum. The residue was stirred with ether. The solvent was carefully decanted. The solid material was dissolved in a mixture of CH₃CN and H₂O. The resulting mixture was frozen and lyophilized for 12.0 h to afford the title compound (46.15 mg, 0.061 mmol, 50.4% yield) as a white solid. ¹H NMR (400 MHz, MeOH-d₄) δ 8.73 (s, 1H), 8.48 (s, 1H), 8.26 (d, J=8.5 Hz, 1H), 7.81 (s, 1H), 7.44 (d, J=8.5 Hz, 1H), 4.80 (br. s., 2H), 4.34 (d, J=16.6 Hz, 1H), 4.04-3.94 (m, 3H), 3.78-3.81 (m, 2H), 3.13 (br. s., 1H), 2.83 (dt, J=14.2, 7.2 Hz, 1H), 2.69 (s, 6H), 2.25-2.15 (m, 7H), 1.39 (t, J=6.5 Hz, 6H). LC-MS (ES): m/z=516.4 [M+H]⁺; HPLC RT and purity: method A=5.703 min and 99.96% and method B=6.004 min and 99.17%.

### Examples 6 and 7

### 1-((1-Aminocyclopropane-1-carbonyl)oxy)ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate ditrifluoroacetate

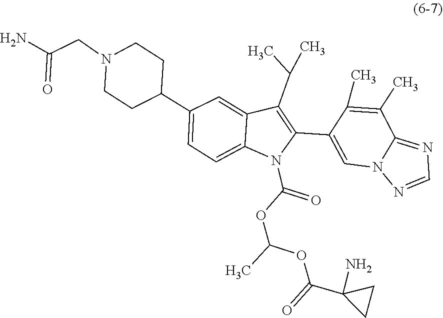 [SMILES: Cc1c(-c2c(C(C)C)c3cc(C4CCN(CC(N)=O)CC4)ccc3n2C(=O)OC(C)OC(=O)C2(N)CC2)cn2ncnc2c1C]

### Intermediate 6A: 1-Chloroethyl 5-(1-(tert-butoxycarbonyl)piperidin-4-yl)-2-(7,8-dimethyl-[1,2,4]triazolo[1,5-a]pyridin-6-yl)-3-isopropyl-1H-indole-1-carboxylate

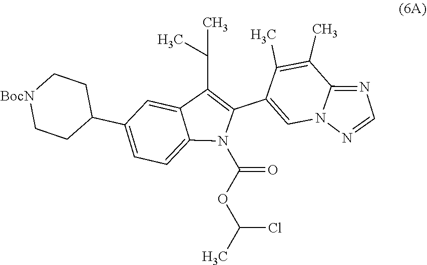 [SMILES: Cc1c(-c2c(C(C)C)c3cc(C4CCN(C(=O)OC(C)(C)C)CC4)ccc3n2C(=O)OC(C)Cl)cn2ncnc2c1C]

To a solution of tert-butyl 4-(2-(7,8-dimethyl-[1,2,4]triazolo[1,5-a]pyridin-6-yl)-3-isopropyl-1H-indol-5-yl)piperidine-1-carboxylate (2.0 g, 4.10 mmol) in THF (40 mL), was added 1 M solution of LiHMDS (6.15 mL, 6.15 mmol) in THF. The reaction mixture was stirred at −70° C. for 15 min under N2. Then 1-chloroethyl chloroformate (0.888 mL, 8.20 mmol) was added. The reaction mixture was stirred at −70° C. for 1 h under N2. The reaction mixture was partitioned between H₂O and EtOAc. The organic layer was washed with H₂O and brine, dried over anhydrous Na₂SO₄ and concentrated to afford the crude product, which was purified by RP HPLC (SunFire OBD (250×30) mm, 5 micron; mobile phase A: 10 mM NH₄OAc in H₂O; mobile phase B: CH₃CN; flow rate: 30 mL/min; gradient (Time (min)/% B): 15/85) to afford two isomers.

Isomer 1: The product was obtained as a white solid (1.0 g; 20.5%). ¹H NMR (400 MHz, DMSO-d₆) δ 8.85 (s, 1H), 8.47 (s, 1H), 8.14 (d, J=9.0 Hz, 1H), 7.73-7.67 (m, 1H), 7.35 (dd, J=8.5, 1.5 Hz, 1H), 6.63-6.55 (m, 1H), 4.12 (s, 1H), 4.15 (s, 1H), 2.93-2.74 (m, 4H), 2.58 (s, 3H), 2.10 (s, 3H), 1.82-1.85 (m, 2H), 1.62 (dd, J=12.3, 3.8 Hz, 2H), 1.44 (s, 9H), 1.34 (d, J=5.5 Hz, 3H), 1.30 (dd, J=7.0, 4.0 Hz, 6H). LC-MS (ES): m/z=594.2 [M+H]⁺.

Isomer 2: The product was obtained as a white solid (0.9 g; 18.5%). ¹H NMR (400 MHz, DMSO-d₆) δ 8.82 (s, 1H), 8.47 (s, 1H), 8.16 (d, J=8.5 Hz, 1H), 7.71 (s, 1H), 7.35 (m, 1H), 6.58-6.50 (m, 1H), 4.15 (br. s., 2H), 2.95-2.75 (m, 3H), 2.59 (s, 4H), 2.08 (s, 3H), 1.86-1.83 (m, 2H), 1.68-1.54 (m, 2H), 1.44 (s, 9H), 1.30 (t, J=7.3 Hz, 6H), 1.14 (d, J=6.0 Hz, 3H). LC-MS (ES): m/z=594.2 [M+H]⁺.

### Intermediate 6B: 1-Chloroethyl 2-(7,8-dimethyl-[1,2,4]triazolo[1,5-a]pyridin-6-yl)-3-isopropyl-5-(piperidin-4-yl)-1H-indole-1-carboxylate trifluoroacetate

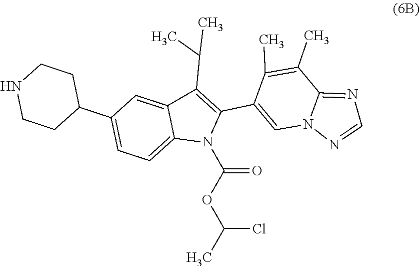 [SMILES: Cc1c(-c2c(C(C)C)c3cc(C4CCNCC4)ccc3n2C(=O)OC(C)Cl)cn2ncnc2c1C]

To the solid of 1-chloroethyl 5-(1-(tert-butoxycarbonyl)piperidin-4-yl)-2-(7,8-dimethyl-[1,2,4]triazolo[1,5-a]pyridin-6-yl)-3-isopropyl-1H-indole-1-carboxylate (1.1 g, 1.851 mmol), Isomer 1 in the previous step, in CH₂Cl₂ (10 mL) at 0° C. under nitrogen atmosphere, was added CF₃CO₂H (1.426 mL, 18.51 mmol). After being stirred at 0° C. for 1 h, the reaction mixture was concentrated under vacuum at 30° C. The residue was stirred with ether. The solvent was carefully decanted. The resultant solid was dried under vacuum to yield crude product as a brown oil (1.1 g, 1.809 mmol, 98% yield). ¹H NMR (400 MHz, DMSO-d₆) δ 8.87 (s, 1H), 8.59 (br. s., 1H), 8.49 (s, 1H), 8.33 (br. s., 1H), 8.19 (d, J=9.0 Hz, 1H), 7.69 (d, J=1.0 Hz, 1H), 7.34 (dd, J=8.8, 1.8 Hz, 1H), 6.59 (q, J=5.5 Hz, 1H), 3.42-3.45 (m, 2H), 3.14-2.97 (m, 3H), 2.85-2.75 (m, 1H), 2.58 (s, 3H), 2.10 (s, 3H), 2.07-1.99 (m, 2H), 1.98-1.83 (m, 2H), 1.39-1.25 (m, 9H). LC-MS (ES): m/z=494.2 [M+H]⁺.

### Intermediate 6C: 1-Chloroethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

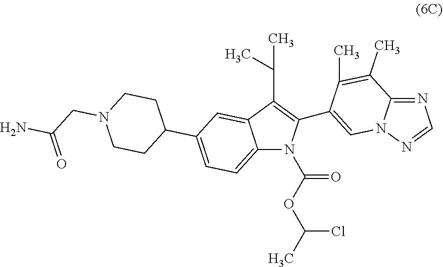 [SMILES: Cc1c(-c2c(C(C)C)c3cc(C4CCN(CC(N)=O)CC4)ccc3n2C(=O)OC(C)Cl)cn2ncnc2c1C]

To a solution of 1-chloroethyl 2-(7,8-dimethyl-[1,2,4]triazolo[1,5-a]pyridin-6-yl)-3-isopropyl-5-(piperidin-4-yl)-1H-indole-1-carboxylate (1.1 g, 2.227 mmol) in DMF (10 mL), were added Et₃N (0.931 mL, 6.68 mmol) and 2-bromoacetamide (0.399 g, 2.89 mmol). The reaction mixture was stirred at room temperature for 1 h. The reaction mixture was diluted with H₂O and extracted with EtOAc. The organic layer was washed with H₂O and brine, dried over anhydrous Na₂SO₄ and concentrated under vacuum at 40° C. to yield the crude product as a white solid, which was purified by RP HPLC (column: SunFire OBD (250×30 mm), 5 micron; mobile phase A: 10 mM NH₄OAc in H₂O; mobile phase B: CH₃CN; flow rate: 30 ml/min; gradient: (time (min)/% B) 0/50, 2/50, 13/80) to afford the title compound (0.8 g, 1.452 mmol, 65.2% yield) as a white solid. ¹H NMR (400 MHz, DMSO-d₆) δ 8.85 (s, 1H), 8.47 (s, 1H), 8.13 (d, J=11.60 Hz, 1H), 7.70 (s, 1H), 7.36 (d, J=11.20 Hz, 1H), 7.26 (s, 1H), 7.16 (s, 1H), 6.57-6.59 (m, 1H), 2.97-2.80 (m, 4H), 2.78-2.65 (m, 2H), 2.57 (s, 3H), 2.15-2.24 (m, 2H), 2.09 (s, 3H), 1.91-1.81 (m, 4H), 1.20-1.34 (m, 9H). LC-MS (ES): m/z=551.2 [M+H]⁺.

### Intermediate 6D: 1-((1-((tert-Butoxycarbonyl)amino)cyclopropane-1-carbonyl)oxy)ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

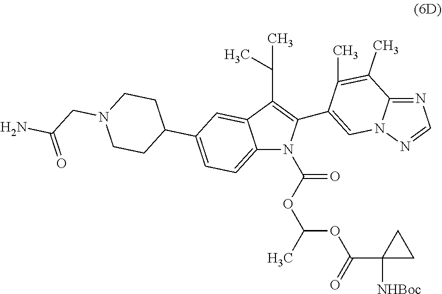 [SMILES: Cc1c(-c2c(C(C)C)c3cc(C4CCN(CC(N)=O)CC4)ccc3n2C(=O)OC(C)OC(=O)C2(NC(=O)OC(C)(C)C)CC2)cn2ncnc2c1C]

To a solution of 1-chloroethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (500 mg, 0.907 mmol) in DMF (5 mL), were added 1-((tert-butoxycarbonyl)amino) cyclopropane-1-carboxylic acid (365 mg, 1.815 mmol), NaI (136 mg, 0.907 mmol), DIPEA (0.475 mL, 2.72 mmol). After being stirred at 75° C. for 12 h under nitrogen atmosphere, the reaction mixture was partitioned between EtOAc and H₂O. The organic layer was washed with brine, dried over anhydrous Na₂SO₄ and concentrated to yield the crude product as a light yellowish solid. The crude product was purified by RP HPLC (column: YMC TRIART C18 (250×4.6) mm, 5 micron; mobile phase: A: 10 mM NH₄OAc in H₂O; B: CH₃CN; flow rate: 1.0 mL/min; gradient (time (min)/% B): 0/30, 20/70, 21/100, 25/100, 26/30) to afford two isomers.

Isomer A: The product was obtained as a white solid (135 mg, 0.187 mmol, 20.58% yield). ¹H NMR (400 MHz, DMSO-d₆) δ 8.79 (s, 1H), 8.46 (s, 1H), 8.10 (d, J=8.5 Hz, 1H), 7.68 (s, 1H), 7.54 (s, 1H), 7.37-7.30 (m, 1H), 7.25 (br. s., 1H), 7.14 (br. s., 1H), 6.65 (d, J=5.0 Hz, 1H), 2.96 (d, J=12.0 Hz, 2H), 2.90 (s, 2H), 2.82-2.71 (m, 1H), 2.70-2.63 (m, 1H), 2.60 (s, 3H), 2.21 (t, J=11.0 Hz, 2H), 2.09 (s, 3H), 1.92-1.75 (m, 4H), 1.36-1.21 (m, 18H), 1.04 (br. s., 2H), 0.98 (d, J=5.5 Hz, 2H). LC-MS (ES): m/z=716.4 [M+H]⁺.

Isomer B: The product was obtained as a white solid (120 mg, 0.159 mmol, 17.55% yield). ¹H NMR (400 MHz, DMSO-d₆) δ 8.76 (s, 1H), 8.46 (s, 1H), 8.07 (d, J=8.5 Hz, 1H), 7.68 (s, 1H), 7.42 (s, 1H), 7.34-7.32 (m, 1H), 7.25 (br. s., 1H), 7.13 (br. s., 1H), 6.70 (d, J=5.0 Hz, 1H), 2.96 (d, J=11.0 Hz, 2H), 2.90 (s, 2H), 2.80-2.74 (m, 1H), 2.72-2.61 (m, 1H), 2.57 (s, 3H), 2.28-2.16 (m, 2H), 2.09 (s, 3H), 1.93-1.76 (m, 4H), 1.37-1.05 (m, 18H), 1.04 (br. s., 2H), 0.98 (d, J=3.0 Hz, 2H). LC-MS (ES): m/z=716.4 [M+H]⁺.

### Example 6

To a solution of Isomer A of 1-((1-((tert-butoxycarbonyl)amino)cyclopropane-1-carbonyl)oxy)ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (10 mg, 0.014 mmol) in CH₂Cl₂ (1 mL), CF₃CO₂H (10.76 μL, 0.140 mmol) was added. The reaction mixture was stirred in ice bath for 1 h and at room temperature for 1 h. The reaction mixture was concentrated under high vacuum pump to yield a residue, which was dissolved in a mixture of CH₃CN and H₂O. The resulting mixture was frozen and lyophilized for 16 h to afford the title compound (5.05 mg, 6.78 μmol, 48.5% yield) as a colorless solid. ¹H NMR (400 MHz, MeOH-d₄) δ 8.64 (s, 1H), 8.46 (s, 1H), 8.22 (d, J=9.0 Hz, 1H), 7.76 (s, 1H), 7.38 (d, J=8.5 Hz, 1H), 6.88 (q, J=5.4 Hz, 1H), 4.03 (s, 2H), 3.78-3.81 (m, 2H), 3.35-3.30 (m, 2H), 3.11 (d, J=7.0 Hz, 1H), 2.88 (dt, J=14.1, 7.0 Hz, 1H), 2.68 (s, 3H), 2.25-2.18 (m, 7H), 1.44-1.35 (m, 8H), 1.32-1.30 (m, 2H), 1.18 (d, J=5.5 Hz, 3H). LC-MS (ES): m/z=616.4 [M+H]⁺. HPLC RT and Purity: method A=7.38 min and 98.78% and method B=9.93 min and 98.96%.

### Example 7

To a solution of Isomer B of 1-((1-((tert-butoxycarbonyl)amino)cyclopropane-1-carbonyl)oxy)ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (120 mg, 0.168 mmol) in dry CH₂Cl₂ (2 mL) at 0° C. under nitrogen atmosphere, was added CF₃CO₂H (0.3 mL, 3.89 mmol). After being stirred for 30 min., the reaction mixture was concentrated under vacuum. The residue was stirred with ether. The solvent was carefully decanted. The resultant solid was dried under vacuum to yield crude product as an off-white solid. The solid was dissolved in a mixture of CH₃CN and H₂O. The resulting mixture was frozen and lyophilized for 12 h to afford the title compound (70 mg, 0.080 mmol, 48.0% yield) as a white solid. ¹H NMR (400 MHz, MeOH-d₄) δ 8.63 (s, 1H), 8.51-8.44 (m, 1H), 8.27 (d, J=8.5 Hz, 1H), 7.76 (s, 1H), 7.39 (d, J=8.5 Hz, 1H), 6.94-6.84 (m, 1H), 4.03 (s, 2H), 3.81-3.78 (m, 2H), 3.17-3.04 (m, 2H), 2.88 (quin, J=7.2 Hz, 1H), 2.85-2.82 (m, 1H), 2.67 (s, 3H), 2.26-2.14 (m, 7H), 1.46-1.26 (m, 9H), 1.24 (d, J=5.5 Hz, 3H), 1.13-1.03 (m, 1H). LC-MS (ES): m/z=616.4 [M+H]⁺. HPLC RT and Purity: method A=7.50 min and 97.86% and method B=9.03 min and 98.63%.

### Example 8 and Example 9

### 6-(5-(1-(2-Amino-2-oxoethyl)piperidin-4-yl)-3-isopropyl-1H-indol-2-yl)-7,8-dimethyl-1-((phosphonooxy)methyl)-[1,2,4]triazolo[1,5-a]pyridin-1-ium trifluoroacetate (8) and 1-(2-Amino-2-oxoethyl)-4-(2-(7,8-dimethyl-[1,2,4]triazolo[1,5-a]pyridin-6-yl)-3-isopropyl-1H-indol-5-yl)-1-((phosphonooxy)methyl)piperidin-1-ium trifluoroacetate (9)

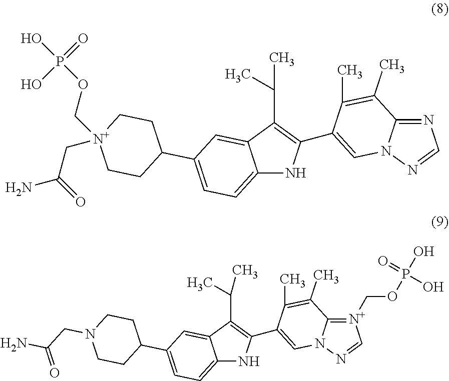 [SMILES: Cc1c(-c2[nH]c3ccc(C4CC[N+](COP(=O)(O)O)(CC(N)=O)CC4)cc3c2C(C)C)cn2ncnc2c1C ; Cc1c(-c2[nH]c3ccc(C4CCN(CC(N)=O)CC4)cc3c2C(C)C)cn2nc[n+](COP(=O)(O)O)c2c1C]

### Intermediates 8A and 9A: (1-(2-Amino-2-oxoethyl)-4-(2-(7,8-dimethyl-[1,2,4]triazolo[1,5-a] pyridin-6-yl)-3-isopropyl-1H-indol-5-yl)piperidin-1-ium-1-yl) methyl tert-butyl phosphate

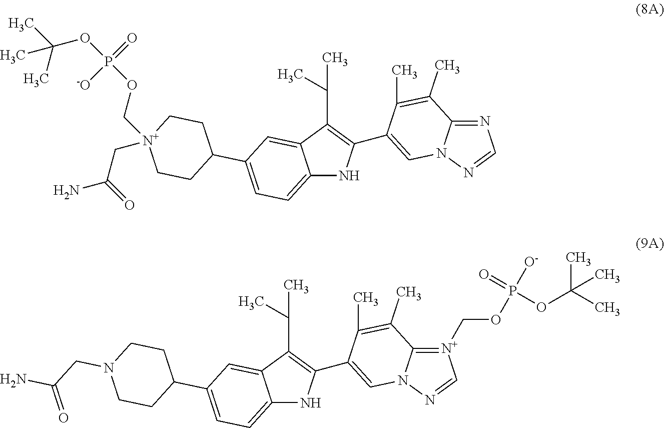

To a stirred solution of 2-(4-(2-(7,8-dimethyl-[1,2,4]triazolo[1,5-a]pyridin-6-yl)-3-isopropyl-1H-indol-5-yl)piperidin-1-yl)acetamide (2.0 g, 4.50 mmol) in CHCl₃ (60.0 mL), K2CO₃ (0.622 g, 4.50 mmol) was added. The reaction mixture was stirred at room temperature for 10 minutes. To this mixture di-tert-butyl (chloromethyl) phosphate (4.07 g, 15.75 mmol) and NaI (2.360 g, 15.75 mmol) were added. The reaction mixture was stirred at 68° C. for 20 h. The reaction mixture was filtered through Celite bed and washed with excess of CH₂Cl₂ and then filtrate was concentrated under reduced pressure.

The crude product was purified by using reverse phase prep. HPLC (column: YMC TRIAT (150×20) mm; 5 micron; mobile phase A: 10 mM NH₄OAc in H₂O; mobile phase B: CH₃CN; flow rate: 20 mL/min; gradient (time (min)/% B): 0/30, 15/50). The fraction was concentrated using high vacuum at 30° C. The residue was dissolved in a mixture of CH₃CN and H₂O, frozen and lyophilized for 16 h to afford the title compound as a mixture of isomers (490 mg, 0.626 mmol, 13.91% yield) as an off-white solid. LC-MS (ES): m/z=611.4 [M+H]⁺.

### Examples 8 and 9

To a stirred solution of 1-(2-amino-2-oxoethyl)-1-(((tert-butoxy(hydroxy) phosphoryl)oxy)methyl)-4-(2-(7,8-dimethyl-[1,2,4]triazolo[1,5-a]pyridin-6-yl)-3-isopropyl-1H-indol-5-yl)piperidin-1-ium iodide (0.6 g, 0.812 mmol) in CH₂Cl₂ (6.0 mL), CF₃CO₂H (0.6 mL, 7.79 mmol) was added at 0° C. The reaction mixture was stirred at same temperature for 30 minutes and then was concentrated under reduced pressure. The crude compound was purified by RP HPLC (column: X-Bridge C18 (250×30) mm; 5 micron; mobile phase A: 0.1% CF₃CO₂H in H₂O; mobile phase B: CH₃CN: MeOH (1:1) flow rate: 30 mL/min; gradient (time (min)/% B): 70/30) to yield two isomers.

Example 8 (Isomer 1): The compound was obtained (86 mg, 0.123 mmol, 15.18% yield) as an off-white solid. ¹H NMR (400 MHz, MeOH-d₄) δ 8.70 (s, 1H), 8.60 (s, 1H), 7.72 (s, 1H), 7.39 (d, J=8.5 Hz, 1H), 7.22 (dd, J=8.5, 1.5 Hz, 1H), 5.33 (d, J=8.0 Hz, 2H), 4.39 (s, 2H), 4.13-4.10 (m, 2H), 3.68-3.56 (m, 2H), 3.17-2.96 (m, 2H), 2.68 (s, 3H), 2.40-2.27 (m, 5H), 2.26-2.16 (m, 2H), 1.41 (d, J=7.5 Hz, 6H). LC-MS (ES): m/z=556.2 [M+H]⁺; HPLC RT and purity: method A=4.574 min and 96.36% and method B=4.245 min and 96.98%.

Example 9 (Isomer 2): The compound was obtained (300 mg, 0.435 mmol, 53.5% yield) as an off-white solid. ¹H NMR (400 MHz, MeOH-d₄) δ 8.71 (s, 1H), 8.62 (s, 1H), 7.70 (s, 1H), 7.37 (d, J=8.5 Hz, 1H), 7.21 (dd, J=8.5, 1.5 Hz, 1H), 5.53 (d, J=8.0 Hz, 2H), 4.20-4.10 (m, 4H), 3.90-3.74 (m, 2H), 3.17-2.93 (m, 2H), 2.68 (s, 3H), 2.49-2.33 (m, 2H), 2.30 (s, 3H), 2.15-2.11 (m, 2H), 1.40 (d, J=7.0 Hz, 6H). LC-MS (ES): m/z=556.2 [M+H]⁺; HPLC RT and purity: method A=4.585 min and 97.19% and method B=4.265 min and 97.74%.

### Example 10

### 4-(5-(1-(2-Amino-2-oxoethyl)piperidin-4-yl)-2-(7,8-dimethyl-[1,2,4]triazolo[1,5-a]pyridin-6-yl)-3-isopropyl-1H-indol-1-yl)-4-oxobutyl dihydrogen phosphate

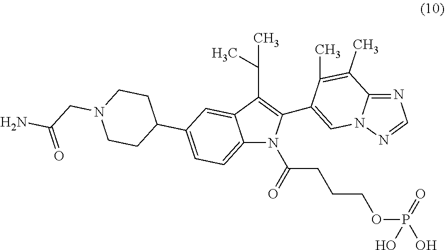 [SMILES: Cc1c(-c2c(C(C)C)c3cc(C4CCN(CC(N)=O)CC4)ccc3n2C(=O)CCCOP(=O)(O)O)cn2ncnc2c1C]

### Intermediate 10A: tert-Butyl 4-(1-(4-((bis(benzyloxy)phosphoryl)oxy)butanoyl)-2-(7,8-dimethyl-[1,2,4]triazolo[1,5-a]pyridin-6-yl)-3-isopropyl-1H-indol-5-yl)piperidine-1-carboxylate

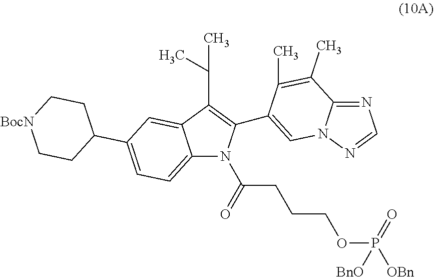 [SMILES: Cc1c(-c2c(C(C)C)c3cc(C4CCN(C(=O)OC(C)(C)C)CC4)ccc3n2C(=O)CCCOP(=O)(OCc2ccccc2)OCc2ccccc2)cn2ncnc2c1C]

To a solution of tert-butyl 4-(2-(7,8-dimethyl-[1,2,4]triazolo[1,5-a]pyridin-6-yl)-3-isopropyl-1H-indol-5-yl)piperidine-1-carboxylate (1 g, 2.051 mmol) in CH₂Cl₂ (10 mL), were added 4-((bis(benzyloxy)phosphoryl)oxy)butanoic acid (1.494 g, 4.10 mmol), DCC (0.635 g, 3.08 mmol) and DMAP (0.376 g, 3.08 mmol). The reaction mixture was stirred for 16 h under N2, and then diluted with CH₂Cl₂. The precipitated solid was filtered and the filtrate was concentrated under vacuum to give the residue. The crude compound was purified by reverse phase HPLC (column: YMC TRIART C18 (150×4.6) mm, 5 micron; mobile phase A: 10 mM NH₄OAc in H₂O; B: CH₃CN; flow rate: 1.0 mL/min; gradient (time (min)/% B: 0/30, 3/60, 15/100, 20/100, 21/30). The fractions were concentrated under high vacuum pump. The final residue was dissolved in CH₃CN and H₂O, and kept for lyophilization for 12 h to obtain the title compound (0.5 g, 0.600 mmol, 29.2% yield). ¹H NMR (400 MHz, DMSO-d₆) δ 8.72 (s, 1H), 8.42 (s, 1H), 8.08 (d, J=8.8 Hz, 1H), 7.63 (s, 1H), 7.40-7.19 (m, 11H), 5.02-4.93 (m, 2H), 4.93-4.83 (m, 4H), 4.09 (d, J=11.2 Hz, 2H), 3.98-3.81 (m, 2H), 3.24-3.09 (m, 2H), 2.83 (t, J=12.0 Hz, 2H), 2.75-2.59 (m, 3H), 2.01 (s, 3H), 1.87-1.58 (m, 6H), 1.57-1.44 (m, 6H), 1.42 (s, 9H). LC-MS (ES): m/z=734.2 [M+H]⁺.

### Intermediate 10B: Dibenzyl (4-(2-(7,8-dimethyl-[1,2,4]triazolo[1,5-a]pyridin-6-yl)-3-isopropyl-5-(piperidin-4-yl)-1H-indol-1-yl)-4-oxobutyl) phosphate trifluoroacetate

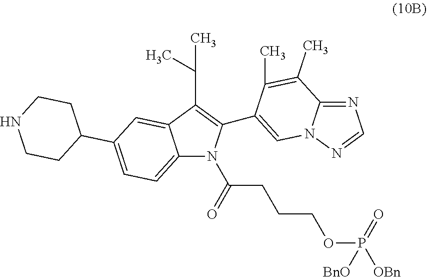 [SMILES: Cc1c(-c2c(C(C)C)c3cc(C4CCNCC4)ccc3n2C(=O)CCCOP(=O)(OCc2ccccc2)OCc2ccccc2)cn2ncnc2c1C]

To a solution of tert-butyl 4-(1-(4-((bis(benzyloxy)phosphoryl)oxy)butanoyl)-2-(7,8-dimethyl-[1,2,4]triazolo[1,5-a]pyridin-6-yl)-3-isopropyl-1H-indol-5-yl)piperidine-1-carboxylate (80 mg, 0.096 mmol) in CH₂Cl₂ (1 mL) at 0° C., was added CF₃CO₂H (0.3 mL, 3.89 mmol). The reaction mixture was stirred at 0° C. for 1 h under N2 and then at room temperature for 1 h. The solvent was removed under high vacuum to afford the title compound (80 mg, 0.094 mmol, 98% yield). LC-MS (ES): m/z=734.2 [M+H]⁺.

### Intermediate 10C: 4-(5-(1-(2-Amino-2-oxoethyl)piperidin-4-yl)-2-(7,8-dimethyl-[1,2,4]triazolo[1,5-a]pyridin-6-yl)-3-isopropyl-1H-indol-1-yl)-4-oxobutyl dibenzyl phosphate

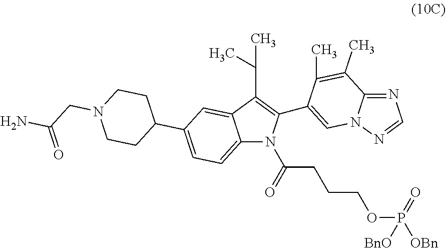 [SMILES: Cc1c(-c2c(C(C)C)c3cc(C4CCN(CC(N)=O)CC4)ccc3n2C(=O)CCCOP(=O)(OCc2ccccc2)OCc2ccccc2)cn2ncnc2c1C]

To a solution of dibenzyl (4-(2-(7,8-dimethyl-[1,2,4]triazolo[1,5-a]pyridin-6-yl)-3-isopropyl-5-(piperidin-4-yl)-1H-indol-1-yl)-4-oxobutyl)phosphate (80 mg, 0.109 mmol) in anhydrous DMF (1 mL), were added 2-bromoacetamide (19.55 mg, 0.142 mmol) and Et₃N (0.046 mL, 0.327 mmol) under a nitrogen atmosphere. The reaction mixture was stirred at room temperature for 5 h and partitioned between H₂O and EtOAc. The organic layer was washed with H₂O and brine, dried over anhydrous Na₂SO₄ and concentrated to yield the crude product as a light yellowish oil, which was purified by RP HPLC (column: SunFire OBD (250×30) mm, 5 micron; mobile phase A: 10 mM NH₄OAc in H₂O; mobile phase B: CH₃CN; flow rate: 30 mL/min) to afford the title compound (70 mg, 0.089 mmol, 81% yield) as a white solid. ¹H NMR (300 MHz, DMSO-d₆) δ 8.80 (s, 1H), 8.45 (s, 1H), 8.13 (d, J=8.8 Hz, 1H), 7.67 (s, 1H), 7.36-7.23 (m, 12H), 7.16 (br. s., 1H), 4.94-4.86 (m, 4H), 3.94-3.82 (m, 2H), 3.00-2.86 (m, 4H), 2.76-2.59 (m, 4H), 2.54 (br. s., 3H), 2.30-2.15 (m, 2H), 2.03 (s, 3H), 1.88 (d, J=11.4 Hz, 2H), 1.79 (br. s., 4H), 1.27 (t, J=6.5 Hz, 6H). LC-MS (ES): m/z=791.8 [M+H]⁺.

### Example 10

To a solution of 4-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-4-oxobutyl dibenzyl phosphate (40 mg, 0.051 mmol) in anhydrous EtOAc (2 mL), was added 10% Pd—C (26.9 mg, 0.025 mmol). The mixture was degassed and then flushed with N2 gas. The reaction mixture was stirred for 1 h under H₂ atmosphere, filtered through Celite bed and the bed was washed with EtOAc. The filtrate was concentrated under vacuum at 30° C. The crude product was purified by RP HPLC (YMC Triart C18 (150×4.6) mm, 5 micron; mobile phase A: 10 mM NH₄OAc in H₂O; mobile phase B: CH₃CN; flow rate: 1.0 mL/min; gradient: (time (min)/% B) 0/10, 20/50, 21/100, 25/100) to afford the title compound (5.23 mg, 8.39 μmol, 16.60% yield) as a white solid. ¹H NMR (300 MHz, DMSO-d₆) δ 8.78 (s, 1H), 8.44 (s, 1H), 8.10 (d, J=8.9 Hz, 1H), 7.66 (s, 1H), 7.37 (br. s., 1H), 7.32-7.19 (m, 2H), 3.64 (br. s., 2H), 3.04 (br. s., 4H), 2.75-2.60 (m, 4H), 2.56 (s, 3H), 2.27-2.33 (m, 2H), 2.06 (s, 3H), 1.89-1.74 (m, 6H), 1.27 (t, J=6.9 Hz, 6H). LC-MS (ES): m/z=609.5 [M−H]⁺; HPLC RT and purity: method A=4.305 min and 99.05% and method B=4.494 min and 98.76%.

### Example 11

### S-(5-(1-(2-Amino-2-oxoethyl)piperidin-4-yl)-2-(7,8-dimethyl-[1,2,4]triazolo[1,5-a]pyridin-6-yl)-3-isopropyl-1H-indol-1-yl)-L-cysteine

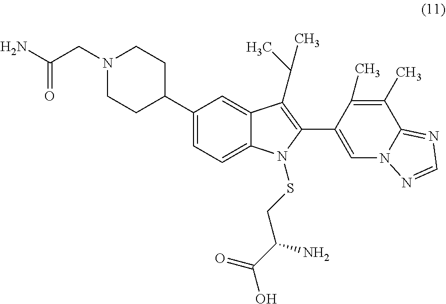 [SMILES: Cc1c(-c2c(C(C)C)c3cc(C4CCN(CC(N)=O)CC4)ccc3n2SC[C@H](N)C(=O)O)cn2ncnc2c1C]

### Intermediate 11A: tert-Butyl S-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-N-(tert-butoxycarbonyl)-L-cysteinate

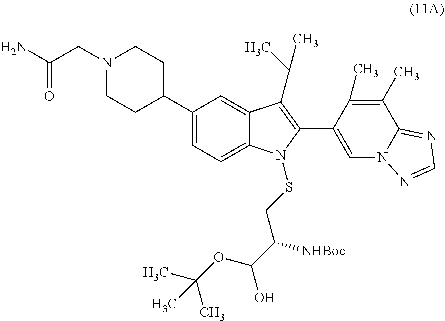 [SMILES: Cc1c(-c2c(C(C)C)c3cc(C4CCN(CC(N)=O)CC4)ccc3n2SC[C@H](NC(=O)OC(C)(C)C)C(O)OC(C)(C)C)cn2ncnc2c1C]

To a solution of 2-(4-(2-(7,8-dimethyl-[1,2,4]triazolo[1,5-a]pyridin-6-yl)-3-isopropyl-1H-indol-5-yl)piperidin-1-yl)acetamide (110 mg, 0.247 mmol) in MeOH (5 mL), were added AgNO₃ (168 mg, 0.990 mmol), Et₃N (0.138 mL, 0.990 mmol) and di-tert-butyl 3,3′-disulfanediyl(2R,2′R)-bis(2-((tert-butoxycarbonyl)amino)propanoate) (547 mg, 0.990 mmol). The reaction mixture was stirred at room temperature for 16 h. The solid was filtered and filtrate was concentrated under reduced pressure to yield an orange colored solid. The crude material was purified by reverse phase prep. HPLC (column: YMC TRIAT (150×20) mm; 5 micron; mobile phase A: 10 mM NH₄OAc in H₂O; mobile phase B: CH₃CN; flow rate: 20 mL/min; gradient (time (min)/% B): 0/50, 14/80). The purification fractions were concentrated under vacuum to yield the residue, which was dissolved in a mixture of CH₃CN and H₂O. The resulting mixture was frozen and lyophilized for 12 h to obtain the title compound (70 mg, 0.097 mmol, 39.3% yield) as a colorless solid. ¹H NMR (400 MHz, MeOH-d₄) δ 8.60 (s, 1H), 8.41 (s, 1H), 7.62 (s, 1H), 7.30 (d, J=8.5 Hz, 1H), 7.14-7.09 (m, 1H), 4.29 (dd, J=8.8, 4.3 Hz, 1H), 3.26-3.19 (m, 3H), 3.06 (br. s., 2H), 3.02-2.91 (m, 1H), 2.83 (dd, J=14.6, 9.0 Hz, 1H), 2.71-2.61 (m, 4H), 2.46-2.35 (m, 2H), 2.27 (s, 3H), 2.13-2.00 (m, 2H), 1.99-1.88 (m, 2H), 1.55-1.43 (m, 18H), 1.40 (d, J=7.0 Hz, 6H). LC-MS (ES): m/z=720.4 [M+H]⁺; HPLC RT and purity: method A=9.046 min and 99.42% and method B=8.294 min and 99.66%.

### Example 11

To a solution of tert-butyl (5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-N-(tert-butoxycarbonyl)-L-cysteinate (70 mg, 0.097 mmol) in CH₂Cl₂ (1 mL), was added CF₃CO₂H (0.075 mL, 0.972 mmol). The reaction mixture was stirred in ice bath for 1 h and then at room temperature for 1 h. The reaction mixture was dried under high vacuum pump to give the residue, which was dissolved in a mixture of CH₃CN and H₂O. The resulting mixture was frozen and lyophilized for 16 h to afford the title compound (52.62 mg, 0.075 mmol, 77% yield) as a colorless solid. ¹H NMR (400 MHz, MeOH-d₄) δ 8.61 (s, 1H), 8.44 (s, 1H), 7.67 (s, 1H), 7.37 (d, J=8.0 Hz, 1H), 7.12 (d, J=7.0 Hz, 1H), 4.19-4.10 (m, 3H), 3.80 (t, J=13.8 Hz, 2H), 3.45-3.20 (m, 4H), 3.06-2.95 (m, 2H), 2.67 (s, 3H), 2.27 (s, 3H), 2.22 (br. s., 4H), 1.41 (d, J=7.0 Hz, 6H). LC-MS (ES): m/z=564.5 [M+H]⁺; HPLC RT and purity: method A=4.13 min and 97.63% and method B=4.95 min and 96.63%.

### Example 12 and Example 13

### 1-(2-Amino-2-oxoethyl)-4-(2-(7,8-dimethyl-[1,2,4]triazolo[1,5-a]pyridin-6-yl)-3-isopropyl-1H-indol-5-yl)-1-(((methyl(3-(((methylglycyl)oxy)methyl)pyridin-2-yl) carbamoyl)oxy)methyl)piperidin-1-ium ditrifluoroacetate (12) and 6-(5-(1-(2-Amino-2-oxoethyl)piperidin-4-yl)-3-isopropyl-1H-indol-2-yl)-7,8-dimethyl-1-(((methyl(3-(((methylglycyl)oxy)methyl)pyridin-2-yl)carbamoyl)oxy)methyl)-[1,2,4]triazolo[1,5-a]pyridin-1-ium tritrifluoroacetate

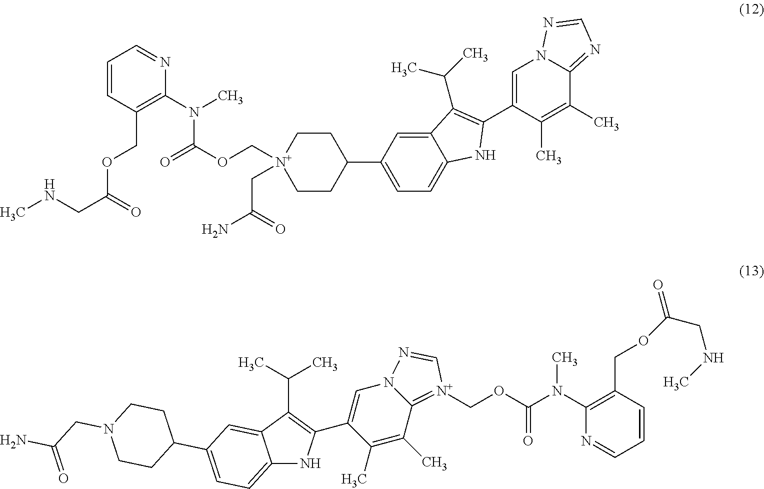

### Intermediates 12A and 13A: 1-(2-Amino-2-oxoethyl)-1-((((3-(((N-(tert-butoxycarbonyl)-N-methylglycyl)oxy)methyl)pyridin-2-yl)(methyl)carbamoyl)oxy)methyl)-4-(2-(7,8-dimethyl-[1,2,4]triazolo[1,5-a]pyridin-6-yl)-3-isopropyl-1H-indol-5-yl)piperidin-1-ium chloride (12A) and 6-(5-(1-(2-Amino-2-oxoethyl)piperidin-4-yl)-3-isopropyl-1H-indol-2-yl)-1-((((3-(((N-(tert-butoxycarbonyl)-N-methylglycyl)oxy)methyl)pyridin-2-yl)(methyl) carbamoyl)oxy)methyl)-7,8-dimethyl-[1,2,4]triazolo[1,5-a]pyridin-1-ium chloride (13A)

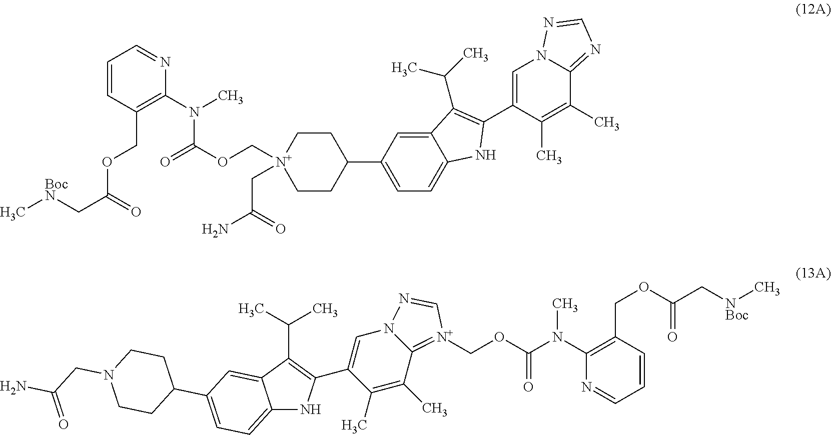

To a stirred solution of 2-(4-(2-(7,8-dimethyl-[1,2,4]triazolo[1,5-a]pyridin-6-yl)-3-isopropyl-1H-indol-5-yl)piperidin-1-yl)acetamide (0.1 g, 0.225 mmol) in CH₃CN (6.0 mL), were added (2-(((chloromethoxy)carbonyl)(methyl)amino)pyridin-3-yl)methyl N-(tert-butoxycarbonyl)-N-methylglycinate (0.271 g, 0.675 mmol) and NaI (0.101 g, 0.675 mmol). The reaction mixture was heated at 68° C. for 16 h. The reaction mixture was concentrated under the reduced pressure and purified by using reverse phase prep. HPLC (column: SunFire C18 (150×21.2) mm 5 micron; mobile Phase A: 0.1% HCO₂H in H₂O; mobile phase B: CH₃CN; flow rate: 20 mL/min; gradient (time (min)/% B): 0/10, 10/35). The fraction was concentrated using high vacuum below 30° C. The residue was dissolved in a mixture of CH₃CN and H₂O, frozen and lyophilized for 16 h to afford 190 mg of the product as an off-white solid. The obtained mixture of isomers was separated by using SFC. (Column/dimensions: Chiralcel OD-H (250×4.6) mm, 5 micron; % Co solvent: 30% of 0.2% Et₃N in MeOH: CH₃CN (1:1); back pressure: 100 bar; temperature: 30° C.). The fractions were concentrated using high vacuum below 30° C. The residues were dissolved separately in a mixture of CH₃CN and H₂O, frozen and lyophilized for 16 h to get two products.

Intermediate 12A (Isomer A): The product was obtained (56 mg, 0.062 mmol, 27.4% yield) as an off-white solid. LC-MS (ES): m/z=811.4 [M+H]⁺.

Intermediate 13A (Isomer B): The product was obtained (24 mg, 0.025 mmol, 11.09% yield) as an off-white solid. LC-MS (ES): m/z=811.4 [M+H]⁺;

### Example 12

To a stirred solution of 1-(2-amino-2-oxoethyl)-1-((((3-(((N-(tert-butoxycarbonyl)-N-methylglycyl)oxy)methyl)pyridin-2-yl)(methyl)carbamoyl)oxy) methyl)-4-(2-(7,8-dimethyl-[1,2,4]triazolo[1,5-a]pyridin-6-yl)-3-isopropyl-1H-indol-5-yl)piperidin-1-ium chloride (50.0 mg, 0.059 mmol) in CH₂Cl₂ (2.0 mL), was added CF₃CO₂H (0.5 mL, 6.49 mmol) at 0° C. The reaction mixture was stirred at the same temperature for 30 minutes and then concentrated under the reduced pressure. The crude compound was triturated with Et₂O. The resultant solid was dissolved in a mixture of CH₃CN:H₂O, frozen and lyophilized for 16 h to yield the title compound (28 mg, 0.028 mmol, 48.0% yield) as a light yellowish solid. ¹H NMR (400 MHz, MeOH-d₄) δ 8.50 (br. s., 2H), 8.33 (s, 1H), 8.00 (br. s., 1H), 7.58 (br. s., 1H), 7.45 (br. s., 1H), 7.32-7.20 (m, J=8.1 Hz, 1H), 7.12-7.00 (m, J=8.6 Hz, 1H), 6.30-5.60 (m, 2H), 5.40-5.20 (m, 2H), 4.40-4.10 (m, 4H), 3.99-3.80 (m, 2H), 3.78-3.70 (m, 2H), 3.64-3.45 (m, 3H), 3.20-3.00 (m, 2H), 2.80 (s, 3H), 2.68 (br. s., 3H), 2.37 (br. s., 2H), 2.20-1.93 (m, 5H), 1.35-1.22 (m, 6H). LC-MS (ES): m/z=711.2 [M+H]⁺. HPLC RT and purity: method A=3.901 min and 97.89% and method B=6.590 min and 95.44%.

### Example 13

To a stirred solution of 6-(5-(1-(2-amino-2-oxoethyl)piperidin-4-yl)-3-isopropyl-1H-indol-2-yl)-1-((((3-(((N-(tert-butoxycarbonyl)-N-methylglycyl)oxy)methyl)pyridin-2-yl)(methyl)carbamoyl)oxy)methyl)-7,8-dimethyl-[1,2,4]triazolo[1,5-a]pyridin-1-ium (20.0 mg, 0.025 mmol) in CH₂Cl₂ (1.0 mL), was added CF₃CO₂H (0.2 mL, 2.60 mmol) at 0° C. The reaction mixture was stirred at the same temperature for 1 h. The mixture was concentrated under the reduced pressure. The crude compound was triturated with Et₂O and the solid was dissolved in a mixture of CH₃CN:H₂O, then frozen and lyophilized for 16 h to yield the product as a light yellow solid. The crude compound was purified by using reverse phase prep. HPLC (column: Inersil ODS C18 (250×4.6 mm), 5 micron; mobile Phase-A: 0.1% CF₃CO₂H in H₂O; mobile Phase-B: CH₃CN; flow rate: 1.0 ml/min; gradient (time (min)/% B): 0/20, 2/25, 20/35, 21/20). The fractions were concentrated using high vacuum at 30° C. The residues were dissolved in a mixture of CH₃CN:H₂O, then frozen and lyophilized for 16 h to afford the title compound (9.4 mg, 9.51 μmol, 38.6% yield) as a brown solid. ¹H NMR (400 MHz, MeOH-d₄) δ 8.68-8.54 (m, 2H), 8.45 (s, 1H), 8.11-8.14 (m, 1H), 7.68-7.73 (m, 1H), 7.60 (br. s., 1H), 7.37 (br. s., 1H), 7.20-7.24 (m, 1H), 6.10-5.50 (m, 2H), 5.40-5.25 (m, 2H), 4.55-4.36 (m, 1H), 4.23 (br. s., 1H), 4.11 (br. s., 2H), 3.90 (br. s., 2H), 3.74 (br. s., 1H), 3.63-3.45 (m, 4H), 3.08-2.96 (m, 2H), 2.80 (s, 3H), 2.67 (s, 3H), 2.38-2.15 (m, 7H), 1.41 (d, J=7.0 Hz, 6H). LC-MS (ES): m/z=711.4 [M+H]⁺; HPLC RT and purity: method A=4.822 min and 95.69% and method B=5.774 min and 95.25%.

### Example 14

### 1-(2-Amino-2-oxoethyl)-4-(2-(7,8-dimethyl-[1,2,4]triazolo[1,5-a]pyridin-6-yl)-3-isopropyl-1H-indol-5-yl)-1-(1-((methyl(3-(((methylglycyl)oxy)methyl)pyridin-2-yl) carbamoyl)oxy)ethyl)piperidin-1-ium ditrifluoroacetate

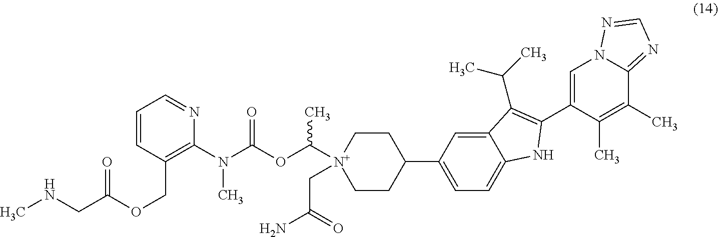

### Intermediate 14A: 1-(2-Amino-2-oxoethyl)-1-(1-(((3-(((N-(tert-butoxycarbonyl)-N-methylglycyl)oxy)methyl)pyridin-2-yl)(methyl)carbamoyl)oxy)ethyl)-4-(2-(7,8-dimethyl-[1,2,4]triazolo[1,5-a]pyridin-6-yl)-3-isopropyl-1H-indol-5-yl)piperidin-1-ium chloride

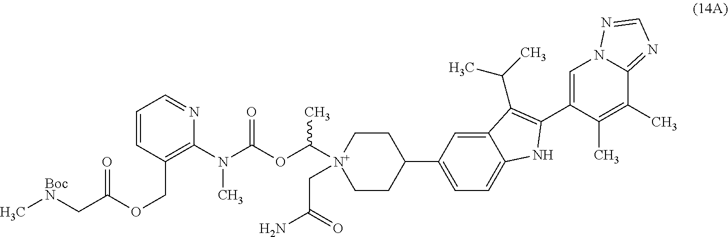

NaI (0.202 g, 1.350 mmol) was added to a stirred solution of 2-(4-(2-(7,8-dimethyl-[1,2,4]triazolo[1,5-a]pyridin-6-yl)-3-isopropyl-1H-indol-5-yl)piperidin-1-yl) acetamide (0.1 g, 0.225 mmol) and (2-(((1-chloroethoxy)carbonyl)(methyl)amino) pyridin-3-yl)methyl N-(tert-butoxycarbonyl)-N-methylglycinate (0.561 g, 1.350 mmol) in CH₃CN (6.0 mL). The reaction mixture was heated to 68° C. and stirred at the same temperature for 18 h. The reaction mixture was concentrated under the reduced pressure and purified by using reverse phase prep. HPLC (Xbridge C18 (150×4.6) mm 5 micron; mobile phase A: 0.1% HCOOH in H₂O; mobile phase B: CH₃CN; flow rate: 2.0 mL/minute; gradient (time (min)/% B): 0/10, 20/70, 21/100, 25/100). The fraction was concentrated using high vacuum at 30° C. The residue was dissolved in a mixture of CH₃CN and H₂O, frozen and lyophilized for 16 h to yield the title compound (16 mg, 0.018 mmol, 7.93% yield) as an off-white solid. ¹H NMR (400 MHz, DMSO-d₆) δ 11.17-11.09 (br. s., 1H), 10.14-9.73 (s, 1H), 9.36 (br. s., 1H), 8.49 (s, 2H), 7.99-7.91 (m, 1H), 7.60-7.09 (m, 6H), 5.25-5.12 (br. s., 2H), 4.02 (br. s., 2H), 3.16 (br. s., 2H), 2.95-2.85 (m, 10H), 2.38 (br. s., 6H), 2.25-2.11 (m, 2H), 1.93-1.73 (m, 7H), 1.45-1.19 (m, 15H). LC-MS (ES): m/z=825.4 [M+H]⁺.

### Example 14

To a stirred solution of 1-(2-amino-2-oxoethyl)-1-(1-(((3-(((N-(tert-butoxycarbonyl)-N-methylglycyl)oxy)methyl)pyridin-2-yl)(methyl)carbamoyl)oxy) ethyl)-4-(2-(7,8-dimethyl-[1,2,4]triazolo[1,5-a]pyridin-6-yl)-3-isopropyl-1H-indol-5-yl) piperidin-1-ium chloride (14.0 mg, 0.016 mmol) in CH₂Cl₂ (1.0 mL), was added CF₃CO₂H (0.1 mL, 1.298 mmol) at 0° C. The reaction mixture was stirred at the same temperature for 45 minutes. The reaction mixture was concentrated under the reduced pressure. The crude compound was triturated with Et₂O. The precipitated solid was dissolved in a mixture of CH₃CN and H₂O, then frozen and lyophilized for 16 h to yield the title compound (12 mg, 0.013 mmol, 79% yield) as an off-white solid. ¹H NMR (400 MHz, MeOH-d₄) δ 9.89-9.39 (m, 1H), 9.14 (s, 1H), 8.54-8.48 (m, 1H), 8.12-8.04 (m, 1H), 7.70 (s, 1H), 7.60 (br. s., 1H), 7.41 (d, J=8.0 Hz, 2H), 7.19-7.15 (m, 1H), 5.43 (br. s., 1H), 5.31 (d, J=12.0 Hz, 1H), 4.18-4.06 (m, 2H), 4.02 (s, 2H), 3.79-3.77 (m, 2H), 3.45 (br. s., 2H), 3.31-3.20 (m, 3H), 3.04-2.88 (m, 5H), 2.79 (br. s., 3H), 2.48 (br. s., 3H), 2.24-2.19 (m, 5H), 1.94 (br. s., 2H), 1.50-1.36 (m, 6H). LC-MS (ES): m/z=725.4 [M+H]⁺. HPLC RT and purity: method A=4.473 min and 94.36% and method B=9.070 min and 94.19%.

### Example 15 ((1-Aminocyclopropane-1-carbonyl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate trifluoroacetate

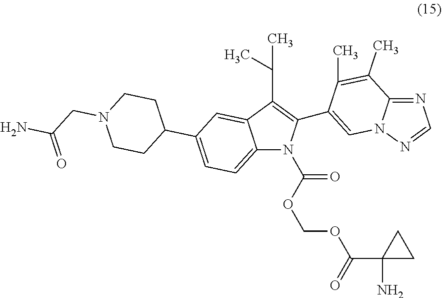 [SMILES: Cc1c(-c2c(C(C)C)c3cc(C4CCN(CC(N)=O)CC4)ccc3n2C(=O)OCOC(=O)C2(N)CC2)cn2ncnc2c1C]

### Intermediate 15: Chloromethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

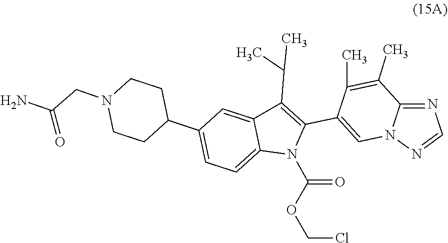 [SMILES: Cc1c(-c2c(C(C)C)c3cc(C4CCN(CC(N)=O)CC4)ccc3n2C(=O)OCCl)cn2ncnc2c1C]

To a solution of 2-(4-(2-(7,8-dimethyl-[1,2,4]triazolo[1,5-a]pyridin-6-yl)-3-isopropyl-1H-indol-5-yl)piperidin-1-yl)acetamide (200 mg, 0.450 mmol) in DMF (2.0 mL) and sodium hydride (54.0 mg, 1.350 mmol), was added chloromethyl chloroformate (0.060 mL, 0.675 mmol). The reaction mixture was stirred at 0° C. for 1 h. The reaction mixture was stirred at 0° C. for 1 h under N2. The reaction mixture was diluted with EtOAc (30 mL). The solution was washed with H₂O (2×20 mL) and brine, dried over anhydrous Na₂SO₄, filtered and concentrated (<40° C.) to yield a colorless liquid. The crude product was purified by RP HPLC. The HPLC fraction was concentrated under vacuum to give a residue, which was dissolved in a mixture of CH₃CN and H₂O. The resulting mixture was frozen and lyophilized for 12 h to obtain the title compound (150 mg, 0.279 mmol, 62.1% yield) as a colorless solid. ¹H NMR (400 MHz, MeOH-d₄) δ 8.60 (s, 1H), 8.41 (s, 1H), 8.20 (d, J=9.0 Hz, 1H), 7.72 (s, 1H), 7.37 (dd, J=8.8, 1.8 Hz, 1H), 5.88 (d, J=6.0 Hz, 1H), 5.74 (d, J=6.5 Hz, 1H), 3.16-3.08 (m, 4H), 2.88 (dt, J=14.1, 7.0 Hz, 1H), 2.73 (d, J=11.5 Hz, 1H), 2.66 (s, 3H), 2.47-2.36 (m, 2H), 2.20 (s, 3H), 2.03-1.91 (m, 4H), 1.38 (dd, J=8.5, 7.0 Hz, 6H). LC-MS (ES): m/z=537.2 [M+H]⁺.

### Intermediate 15B: ((1-((tert-Butoxycarbonyl)amino)cyclopropane-1-carbonyl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

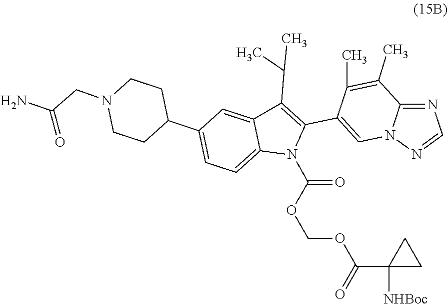 [SMILES: Cc1c(-c2c(C(C)C)c3cc(C4CCN(CC(N)=O)CC4)ccc3n2C(=O)OCOC(=O)C2(NC(=O)OC(C)(C)C)CC2)cn2ncnc2c1C]

To a solution of chloromethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (70 mg, 0.130 mmol) and 1-((tert-butoxycarbonyl)amino)cyclopropane-1-carboxylic acid (26.2 mg, 0.130 mmol) in DMF, was added KI (21.64 mg, 0.130 mmol). The reaction mixture was stirred at room temperature for 16 h. Water was added to make a homogeneous solution, which was directly purified by RP HPLC (column: YMC TRAIRT (150×20 mm), 5 micron; mobile phase A: 10 mM NH₄OAc in H₂O; mobile phase B: CH₃CN; flow rate: 20 mL/min; gradient (time (min)/% B): 0/20, 2/40, 15/80, 17/100). After purification, the fraction was concentrated under reduced pressure. The residue was dissolved in CH₃CN and H₂O and the mixture was subjected for lyophilization to yield the title compound (30 mg, 0.043 mmol, 32.8% yield) as a white solid. ¹H NMR (400 MHz, MeOH-d₄) δ 8.57 (s, 1H), 8.40 (s, 1H), 8.22 (d, J=8.5 Hz, 1H), 7.70 (s, 1H), 7.35 (d, J=7.0 Hz, 1H), 5.72-5.62 (m, 2H), 3.20-3.11 (m, 4H), 2.88-2.69 (m, 2H), 2.66 (s, 3H), 2.45 (t, J=11.3 Hz, 2H), 2.18 (s, 3H), 2.06-1.91 (m, 4H), 1.43-1.33 (m, 15H), 1.26 (br. s., 2H), 1.09 (d, J=3.5 Hz, 2H). LC-MS (ES): m/z=702.4 [M+H]⁺.

### Example 15

To a solution of ((1-((tert-butoxycarbonyl)amino)cyclopropane-1-carbonyl)oxy) methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (20 mg, 0.028 mmol) in CH₂Cl₂ (1 mL), was added CF₃CO₂H (0.2 mL, 0.570 mmol). The reaction mixture was stirred in ice bath for 1 h and then at room temperature for 1 h. The crude reaction mixture was concentrated under high vacuum to give a residue. The residue was dissolved in a mixture of CH₃CN and H₂O. The resulting mixture was frozen and lyophilized for 2 days to obtain the title compound (15.76 mg, 0.021 mmol, 72.6% yield) as a colorless solid. ¹H NMR (400 MHz, MeOH-d₄) δ 8.56 (s, 1H), 8.44 (s, 1H), 8.27 (d, J=8.5 Hz, 1H), 7.76 (s, 1H), 7.38 (d, J=9.0 Hz, 1H), 5.87 (d, J=6.0 Hz, 1H), 5.74 (d, J=6.0 Hz, 1H), 4.03 (s, 2H), 3.81-3.78 (m, 2H), 3.31-3.25 (m, 2H), 3.10 (br. s., 1H), 2.87 (t, J=7.3 Hz, 1H), 2.67 (s, 3H), 2.27-2.16 (m, 7H), 1.51-1.44 (m, 2H), 1.41-1.33 (m, 8H). LC-MS (ES): m/z=602.3 [M+H]⁺. HPLC RT and purity: method A=5.03 min and 95.04% and method B=9.03 min and 94.19%.

### Example 16 (Phosphonooxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

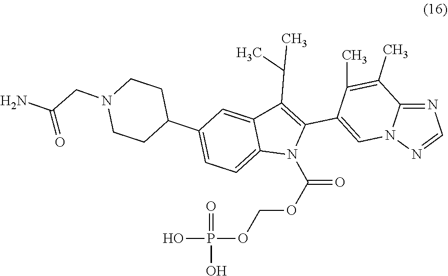 [SMILES: Cc1c(-c2c(C(C)C)c3cc(C4CCN(CC(N)=O)CC4)ccc3n2C(=O)OCOP(=O)(O)O)cn2ncnc2c1C]

### Intermediate 16A: ((Di-tert-butoxyphosphoryl)oxy)methyl 5-(1-(2-amino-2-oxoethyl) piperidin-4-yl)-2-(7,8-dimethyl-[1,2,4]triazolo[1,5-a]pyridin-6-yl)-3-isopropyl-1H-indole-1-carboxylate

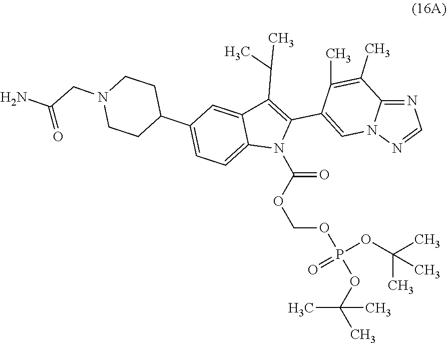 [SMILES: Cc1c(-c2c(C(C)C)c3cc(C4CCN(CC(N)=O)CC4)ccc3n2C(=O)OCOP(=O)(OC(C)(C)C)OC(C)(C)C)cn2ncnc2c1C]

To a solution of chloromethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (120 mg, 0.223 mmol) in DMF (1 mL), were added tetra-n-butyl ammonium di-tert-butyl phosphate (202 mg, 0.447 mmol) and KI (37.1 mg, 0.223 mmol). The reaction mixture was stirred at room temperature for 16 h. Water was added to the reaction mixture, yielding a homogenous solution, which was purified by RP HPLC (column: X-Bridge C18 (150×19) mm; 5 micron; mobile phase A: 10 mM NH₄OAc in H₂O; mobile phase B: CH₃CN:IPA (70:30); flow rate: 20 mL/min; gradient: 0/30, 15/60). After purification, the fraction was concentrated under reduced pressure and the residue was dissolved in CH₃CN and H₂O. The mixture was subjected to lyophilization to afford the title compound (25 mg, 0.035 mmol, 15.74% yield). ¹H NMR (400 MHz, MeOH-d₄) δ 8.55-8.52 (m, 1H), 8.42-8.38 (m, 1H), 8.36-8.25 (m, 1H), 7.73-7.69 (m, 1H), 7.36 (t, J=7.3 Hz, 1H), 5.66 (dd, J=13.3, 3.3 Hz, 2H), 3.72 (br. s., 1H), 3.50 (br. s., 2H), 2.88 (td, J=7.0, 4.0 Hz, 4H), 2.75-2.71 (m, 1H), 2.65 (s, 3H), 2.20 (s, 3H), 2.12 (br. s., 2H), 2.06 (br. s., 2H), 1.46-1.30 (m, 24H). LC-MS (ES): m/z=711.2 [M+H]⁺.

### Example 16

To a solution of ((di-tert-butoxyphosphoryl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (25 mg, 0.035 mmol) in CH₂Cl₂ (1 mL), was added CF₃CO₂H (0.25 mL, 3.24 mmol). The reaction mixture was stirred at room temperature for 1 h. The crude reaction mixture was concentrated under high vacuum pump. The residue was dissolved in a mixture of CH₃CN and H₂O. The resulting mixture was frozen and lyophilized for 2 days to obtain the title compound (14.42 mg, 0.019 mmol, 55.2% yield) as a colorless solid. ¹H NMR (400 MHz, MeOH-d₄) δ 8.62 (s, 1H), 8.55 (s, 1H), 8.34 (d, J=8.5 Hz, 1H), 7.74 (s, 1H), 7.37 (d, J=8.5 Hz, 1H), 5.64 (ddd, J=18.7, 13.7, 5.3 Hz, 2H), 4.02 (s, 2H), 3.81-3.78 (m, 2H), 3.30-3.28 (m, 2H), 3.11-3.05 (m, 1H), 2.93-2.82 (m, 1H), 2.66 (s, 3H), 2.31-2.21 (m, 7H), 1.38 (dd, J=9.0, 7.0 Hz, 6H). LC-MS (ES): m/z=599.0 [M+H]⁺. HPLC RT and Purity: method A=7.77 min and 95.94% and method B=9.14 min and 95.65%.

### Example 17 (5-(1-(2-Amino-2-oxoethyl)piperidin-4-yl)-2-(7,8-dimethyl-[1,2,4]triazolo[1,5-a]pyridin-6-yl)-3-isopropyl-1H-indol-1-yl)phosphonic acid

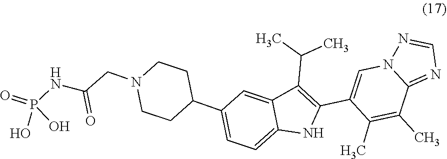 [SMILES: Cc1c(-c2[nH]c3ccc(C4CCN(CC(=O)NP(=O)(O)O)CC4)cc3c2C(C)C)cn2ncnc2c1C]

### Intermediate 17A: Bis(2-(trimethylsilyl)ethyl) (5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl) phosphonate

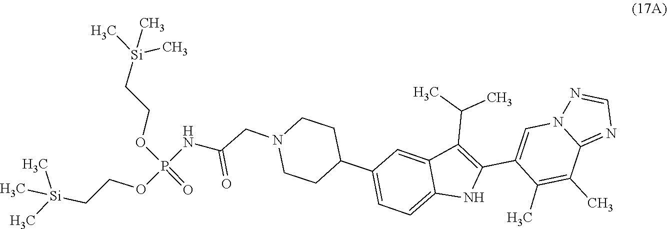 [SMILES: Cc1c(-c2[nH]c3ccc(C4CCN(CC(=O)NP(=O)(OCC[Si](C)(C)C)OCC[Si](C)(C)C)CC4)cc3c2C(C)C)cn2ncnc2c1C]

To a stirred solution of 2-(4-(2-(7,8-dimethyl-[1,2,4]triazolo[1,5-a]pyridin-6-yl)-3-isopropyl-1H-indol-5-yl)piperidin-1-yl)acetamide (0.2 g, 0.450 mmol) in CH₂Cl₂ (2 mL), were added 1H-tetrazole (4% solution in CH₃CN) (1.300 mL, 0.594 mmol) and bis(2-(trimethylsilyl)ethyl)diisopropylphosphoramidite (0.5 mL, 1.215 mmol). The reaction mixture was stirred at room temperature for 16 h. The reaction mixture was cooled to 0° C. A solution of aqueous H₂O₂ (0.2 mL, 1.940 mmol) was added. The reaction mixture was stirred at the same temperature for 10 min. The reaction mixture was partitioned between EtOAc and H₂O. The organic layer was washed with brine, dried over anhydrous Na₂SO₄, filtered and concentrated under vacuum to yield a colorless oil. The crude product was purified by RP HPLC (column: X-Bridge C18 (150×19) mm; 5 micron; mobile phase A: 10 mM NH₄OAc in H₂O; mobile phase B: CH₃CN; flow rate: 18 mL/min; gradient (time (min)/% B): 0/50, 2/50, 15/80). The fraction was concentrated using high vacuum at 30° C. The residue was dissolved in a mixture of CH₃CN and H₂O, frozen and lyophilized for 12 h to yield the title compound (173 mg, 0.234 mmol, 52.0% yield) as a white solid. ¹H NMR (400 MHz, DMSO-d₆) δ 10.99-10.94 (m, 1H), 8.74 (d, J=3.5 Hz, 1H), 8.47 (s, 1H), 7.60-7.52 (m, 1H), 7.35-7.23 (m, 1H), 7.03 (d, J=8.5 Hz, 1H), 4.20-4.07 (m, 3H), 3.86-3.76 (m, 1H), 3.20-3.10 (m, 1H), 3.02-2.83 (m, 3H), 2.64-2.56 (m, 4H), 2.39-2.26 (m, 2H), 2.16 (s, 3H), 2.08 (br. s., 1H), 2.00 (br. s., 1H), 1.81 (br. s., 3H), 1.38-1.28 (m, 6H), 1.26-1.10 (m, 1H), 1.10-0.98 (m, 3H), 0.93 (br. s., 1H), 0.08-0.02 (m, 18H). LC-MS (ES): m/z=725.5 [M+H]⁺.

### Example 17

To a stirred solution of bis(2-(trimethylsilyl)ethyl) (5-(1-(2-amino-2-oxoethyl) piperidin-4-yl)-2-(7,8-dimethyl-[1,2,4]triazolo[1,5-a]pyridin-6-yl)-3-isopropyl-1H-indol-1-yl)phosphonate (100 mg, 0.138 mmol) in CH₂Cl₂ (3 mL) at 0° C., was added CF₃CO₂H (0.5 mL, 6.49 mmol). The reaction mixture was stirred at 0° C. for 10 min, then brought to room temperature and stirred for 1 h. The reaction mixture was concentrated to remove the solvent at 30° C. The residue was purified using RP HPLC (column: X-Bridge phenyl (250×19 mm), 5 micron; mobile phase A: 10 mM NH₄HCO₃ in H₂O pH-9.5; mobile phase B: CH₃CN; flow rate: 16 mL/min; gradient (time (min)/% B): 0/20, 2/20, 9/35). The fraction was concentrated using high vacuum at 30° C. The residue was dissolved in a mixture of CH₃CN and H₂O, frozen and lyophilized for 12 h to afford the title compound (45 mg, 0.081 mmol, 59.1% yield) as a white solid. ¹H NMR (400 MHz, MeOH-d₄) δ 8.60 (s, 1H), 8.40 (s, 1H), 7.65 (s, 1H), 7.34 (d, J=8.5 Hz, 1H), 7.13 (d, J=8.5 Hz, 1H), 4.22 (br. s., 1H), 3.65 (br. s., 1H), 3.43 (br. s., 2H), 3.25-3.18 (m, 1H), 3.04-2.93 (m, 2H), 2.76 (br. s., 1H), 2.66 (s, 3H), 2.27 (s, 3H), 2.12 (br. s., 4H), 1.40 (d, J=7.0 Hz, 6H). LC-MS (ES): m/z=525.3 [M+H]⁺; HPLC RT and purity: method A=6.636 min and 95.01% and method B=8.004 min and 95.29%.

### Example 18

### (5-(1-(2-Amino-2-oxoethyl)piperidin-4-yl)-2-(7,8-dimethyl-[1,2,4]triazolo[1,5-a]pyridin-6-yl)-3-isopropyl-1H-indol-1-yl)phosphonate

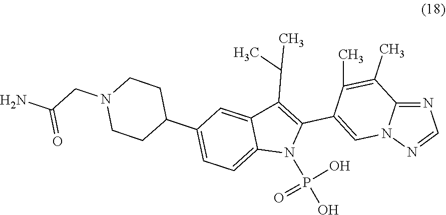

### Intermediate 18A: tert-Butyl 4-(1-(bis(benzyloxy)phosphoryl)-2-(7,8-dimethyl-[1,2,4]triazolo[1,5-a]pyridin-6-yl)-3-isopropyl-1H-indol-5-yl)piperidine-1-carboxylate

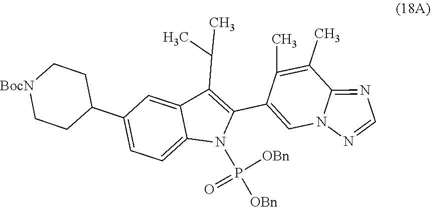

To a stirred solution of tert-butyl 4-(2-(7,8-dimethyl-[1,2,4]triazolo[1,5-a]pyridin-6-yl)-3-isopropyl-1H-indol-5-yl)piperidine-1-carboxylate (0.75 g, 1.538 mmol) in THE (20.0 mL), sodium hydride (0.154 g, 3.84 mmol) was added. The reaction mixture was stirred at room temperature for 30 minutes. To this mixture, tetrabenzyl diphosphate (3.31 g, 6.15 mmol) in THE (5.0 mL) was added at 0° C. The reaction mixture was stirred at same temperature for 1 h., quenched with ice cold H₂O and then partitioned between EtOAc and H₂O. The organic layer was washed with brine solution, dried over Na₂SO₄ and concentrated under reduced pressure to get the residue. The crude product was purified by using reverse phase prep. HPLC (column: YMC TRIART C18 (150×4.6) mm, 5 micron; mobile phase A: 10 mM NH₄OAc in H₂O; mobile phase B: CH₃CN; flow rate: 1.0 mL/min; gradient (time (min)/% B: 0/30, 3/60, 15/100, 20/100, 21/30). The fraction was concentrated using high vacuum below 30° C. The residue was dissolved in CH₂Cl₂ and dried over Na₂SO₄, filtered and concentrated under the reduced pressure to afford the title compound (0.52 g, 0.667 mmol, 43.4% yield) as an off-white solid. ¹H NMR (400 MHz, DMSO-d₆) δ 8.62 (s, 1H), 8.44 (s, 1H), 7.92 (d, J=8.5 Hz, 1H), 7.67-7.61 (m, 1H), 7.34-7.15 (m, 9H), 7.10-7.02 (m, 2H), 5.15-4.94 (m, 2H), 4.90 (d, J=8.0 Hz, 2H), 4.13 (d, J=12.0 Hz, 2H), 2.95-2.75 (m, 3H), 2.71-2.59 (m, 1H), 2.39 (s, 3H), 1.96 (s, 3H), 1.82-1.85 (m, 2H), 1.69-1.55 (m, 2H), 1.44 (s, 9H), 1.25 (dd, J=7.0, 2.0 Hz, 6H). LC-MS (ES): m/z=748.4 [M+H]⁺.

### Intermediate 18B: Dibenzyl (5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)phosphonate

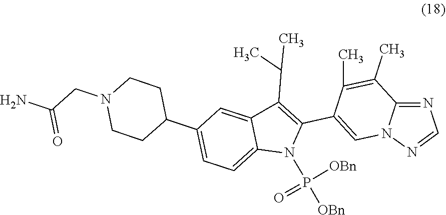

To a stirred solution of tert-butyl 4-(1-(bis(benzyloxy)phosphoryl)-2-(7,8-dimethyl-[1,2,4]triazolo[1,5-a]pyridin-6-yl)-3-isopropyl-1H-indol-5-yl)piperidine-1-carboxylate (510 mg, 0.682 mmol) in CH₂Cl₂ (6.0 mL), CF₃CO₂H (0.6 mL, 7.79 mmol) was added at 0° C. The reaction mixture was stirred at same temperature for 2.5 h. The mixture was concentrated under reduced pressure and co-distilled with CH₂Cl₂ and Et₂O to yield the title compound (1.01 g) as a light brown oil. LC-MS (ES): m/z=648.2 [M+H]⁺. To a stirred solution of above crude product in DMF (5.0 mL) were added Et₃N (0.549 mL, 3.94 mmol) and 2-bromoacetamide (0.235 g, 1.707 mmol). The reaction mixture was stirred at room temperature for 2 h. The reaction mixture was partitioned between EtOAc and H₂O. The organic layer was washed with brine solution and dried over Na₂SO₄, filtered and concentrated under reduced pressure to afford the title compound (0.6 g, 0.851 mmol, 64.8% yield) as a light brown oil. LC-MS (ES): m/z=705.6 [M+H]⁺.

### Example 18

To a stirred solution of dibenzyl (5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)phosphonate (580.0 mg, 0.823 mmol) in ethanol (5.0 mL), Pd/C (150 mg, 0.141 mmol) was added. The reaction mixture was stirred under hydrogen atmosphere at room temperature for 3 h. The reaction mixture was filtered through Celite bed and washed with a mixture of MeOH and H₂O (1:1). The filtrate was concentrated under reduced pressure to yield a residue. The crude material was purified by using reverse phase prep. HPLC (column: YMC TRIAT (150×20) mm, 5 micron; mobile phase A: 10 mM NH₄HCO₃ in H₂O, pH: 9.5 mobile phase B: CH₃CN; flow rate: 20 mL/min; gradient (time (min)/% B): 0/10, 13/35). The fraction was kept as such for lyophilization to yield the title compound (6.0 mg, 10.31 μmol, 1.25% yield) as an off-white solid. ¹H NMR (400 MHz, MeOH-d₄) δ 8.53 (s, 1H), 8.47 (s, 1H), 8.21 (d, J=9.0 Hz, 1H), 7.62 (s, 1H), 7.16 (d, J=8.5 Hz, 1H), 4.00 (s, 2H), 3.74-3.77 (m, 2H), 3.30-3.20 (m, 2H), 3.00 (br. s., 1H), 2.77 (dt, J=14.2, 7.2 Hz, 1H), 2.63 (s, 3H), 2.25 (s, 3H), 2.23-2.13 (m, 4H), 1.35 (dd, J=7.0, 4.5 Hz, 6H). LC-MS (ES): m/z=525.4 [M+H]⁺; HPLC RT and purity: method A=5.901 min and 96.09% and method B=5.794 min and 96.02%.

### Example 19

### (Phosphonooxy)methyl (2-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-2-oxoethyl)(methyl) carbamate

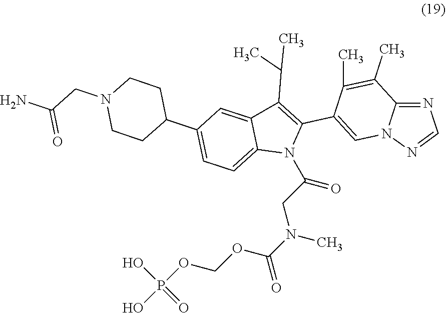 [SMILES: Cc1c(-c2c(C(C)C)c3cc(C4CCN(CC(N)=O)CC4)ccc3n2C(=O)CN(C)C(=O)OCOP(=O)(O)O)cn2ncnc2c1C]

### Intermediate 19A: ((bis(Benzyloxy)phosphoryl)oxy)methyl (2-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-2-oxoethyl)(methyl)carbamate

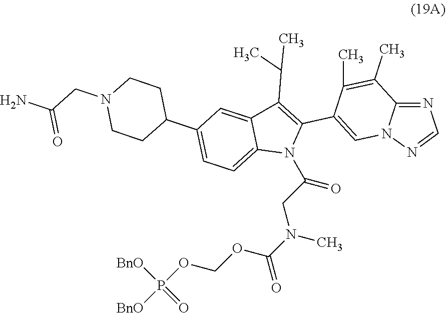 [SMILES: Cc1c(-c2c(C(C)C)c3cc(C4CCN(CC(N)=O)CC4)ccc3n2C(=O)CN(C)C(=O)OCOP(=O)(OCc2ccccc2)OCc2ccccc2)cn2ncnc2c1C]

To a stirred solution of 2-(4-(2-(7,8-dimethyl-[1,2,4]triazolo[1,5-a]pyridin-6-yl)-3-isopropyl-1-(methylglycyl)-1H-indol-5-yl)piperidin-1-yl)acetamide (0.275 g, 0.533 mmol) and ((bis(benzyloxy)phosphoryl)oxy)methyl chloroformate (0.723 g, 0.800 mmol) in THF (5 mL) at 0° C., DIPEA (1.863 mL, 10.67 mmol) was added slowly. The reaction mixture was stirred at 0° C. for 1 h. The reaction mixture was partitioned between H₂O and EtOAc. The organic layer was washed with brine solution, dried over anhydrous Na₂SO₄, filtered and concentrated under vacuum to give crude product as a light yellow oil. The crude product was purified using RP HPLC. The fraction was concentrated using high vacuum at 30° C. The residue was dissolved in a mixture of CH₃CN and H₂O, frozen and lyophilized for 12 h to afford the title product (0.060 g, 0.068 mmol, 12.81% yield). ¹H NMR (400 MHz, DMSO-d₆) δ 8.89-8.85 (m, 1H), 8.50 (d, J=5.0 Hz, 1H), 8.19 (dd, J=8.5, 7.0 Hz, 1H), 7.69 (s, 1H), 7.37-7.23 (m, 11H), 7.19 (dd, J=7.8, 1.8 Hz, 2H), 5.61-5.43 (m, 2H), 5.03 (dd, J=7.8, 5.8 Hz, 2H), 4.98-4.93 (m, 1H), 4.91 (dd, J=8.0, 4.5 Hz, 1H), 4.16-4.06 (m, 1H), 4.03-3.93 (m, 1H), 2.96 (br. s., 3H), 2.73 (s, 1H), 2.77 (s, 2H), 2.72-2.62 (m, 2H), 2.56 (d, J=6.5 Hz, 3H), 2.23 (br. s., 2H), 2.14-2.03 (m, 4H), 1.92 (s, 1H), 1.82 (br. s., 3H), 1.36-1.22 (m, 6H). LC-MS (ES): m/z=850.2 [M+H]⁺.

### Example 19

To a stirred solution of ((bis(benzyloxy)phosphoryl)oxy)methyl (2-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-2-oxoethyl)(methyl)carbamate (0.050 g, 0.059 mmol) in 1,2-dichloroethane (2 mL), were added CF₃CO₂H (0.227 mL, 2.94 mmol) and anisole (0.161 mL, 1.471 mmol) at 0° C. The reaction mixture was stirred at 50° C. for 2 h. The reaction mixture solvent was concentrated under vacuum at 30° C. to yield a brown gum. The residue was triturated with Et₂O and decanted. The precipitated solid was dried under vacuum. The solid was dissolved in a mixture of CH₃CN and H₂O, frozen and lyophilized for 12 h afford the crude product as a light brown solid. The crude product was purified using RP HPLC (column: SunFire C18 (150×19) mm; 5 micron; mobile phase A: 0.1% CF₃CO₂H in H₂O; mobile phase B: CH₃CN; flow rate: 20 mL/min; gradient (time (min)/% B): 0/10, 2/10, 6/30). The fraction was concentrated using high vacuum at 30° C. The residue was dissolved in a mixture of CH₃CN and H₂O, frozen and lyophilized for 12 h to afford the title compound (0.028 g, 0.034 mmol, 58.6% yield) as an off-white solid. ¹H NMR (400 MHz, DMSO-d₆) δ 9.52 (br. s., 1H), 8.85-8.89 (m, 1H), 8.54-8.50 (m, 1H), 8.28 (d, J=8.5 Hz, 1H), 7.98 (s, 1H), 7.73-7.71 (m, 1H), 7.31 (d, J=9.0 Hz, 1H), 5.45-5.28 (m, 2H), 4.22-4.06 (m, 2H), 4.03-3.93 (m, 2H), 3.80-3.60 (m, 2H), 3.30-3.10 (m, 2H), 3.05-2.90 (m, 1H), 2.84-2.79 (m, 3H), 2.70-2.59 (m, 4H), 2.22-1.97 (m, 7H), 1.36-1.23 (m, 6H). LC-MS (ES): m/z=670.2 [M+H]⁺; HPLC RT and purity: method C=4.466 min and 96.47% and method D=7.144 min and 98.68%.

### Examples 20 and 21

### 1-(2-Amino-2-oxoethyl)-4-(2-(7,8-dimethyl-[1,2,4]triazolo[1,5-a]pyridin-6-yl)-3-isopropyl-1H-indol-5-yl)-1-(((methyl(2-(phosphonooxy)ethyl)carbamoyl)oxy)methyl) piperidin-1-ium trifluoroacetate (20) and 6-(5-(1-(2-amino-2-oxoethyl)piperidin-4-yl)-3-isopropyl-1H-indol-2-yl)-7,8-dimethyl-1-(((methyl(2-(phosphonooxy)ethyl)carbamoyl) oxy)methyl)-[1,2,4]triazolo[1,5-a]pyridin-1-ium trifluoroacetate (21)

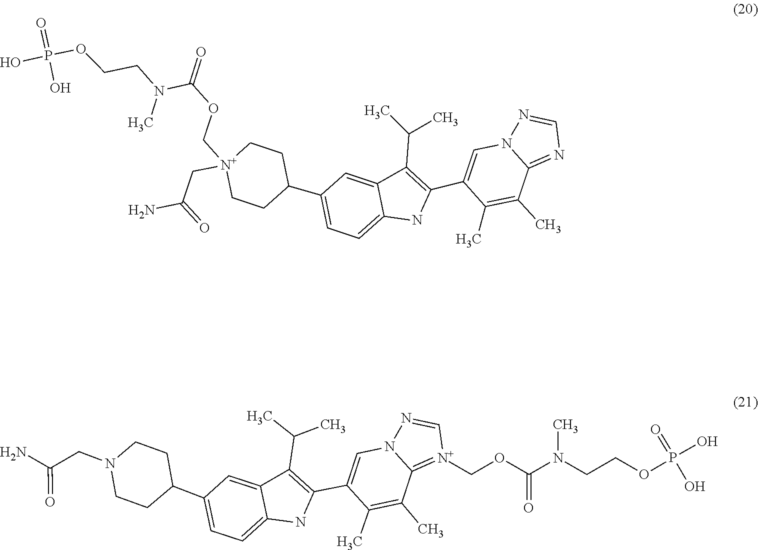

To a solution of 2-(4-(2-(7,8-dimethyl-[1,2,4]triazolo[1,5-a]pyridin-6-yl)-3-isopropyl-1H-indol-5-yl)piperidin-1-yl)acetamide (500 mg, 1.125 mmol) in CHCl₃ (10 mL), were added chloromethyl (2-((bis(benzyloxy)phosphoryl)oxy)ethyl)(methyl) carbamate (1684 mg, 3.94 mmol) and TBAI (1454 mg, 3.94 mmol). The reaction mixture was stirred at 70° C. for 16 h. The reaction mixture was filtered through syringe filter and washed with CH₂Cl₂. The organic layer was concentrated to obtain the crude product as a light yellowish oil, which was purified by reverse phase HPLC (column: SunFire C18 (150×19) mm; 5 micron; mobile phase A: 0.1% HCO₂H in H₂O mobile phase B: CH₃CN; flow rate: 20 mL/min; gradient: 0/10, 20/50 to afford 60 mg of a mixture of two compounds [LC-MS (ES): m/z=746.5 [M+H] and LC-MS (ES): m/z=836.6 [M+H]⁺].

To a stirred solution of the mixture of compounds (60 mg, 0.072 mmol) in 1,2-dichloroethane (2 mL), were added CF₃CO₂H (5.52 μL, 0.072 mmol) and anisole (7.83 μL, 0.072 mmol) at 0° C. The reaction mixture was stirred at 50° C. for 2 h. The reaction mixture was concentrated under vacuum at 30° C. to yield the crude product as a light brown oil. The crude product was purified by RP HPLC (Kinetex PFP (150×21.2) mm; 5 micron; mobile phase A: 0.1% CF₃CO₂H in H₂O mobile phase B: CH₃CN:IPA (70:30); flow rate: 18 mL/min; gradient (time (min)/% B): 0/10, 10/25) to afford Isomer 1 and Isomer 2 as off-white solids.

Example 20 (Isomer 1): 24.25 mg, 0.031 mmol, 43.0% yield): ¹H NMR (400 MHz, DMSO-d₆) δ 8.70 (s, 1H), 8.49 (s, 1H), 7.64 (s, 1H), 7.34 (d, J=8.5 Hz, 1H), 7.21 (d, J=9.0 Hz, 1H), 5.62 (s, 2H), 4.36 (br. s., 2H), 4.06-3.99 (m, 2H), 3.90-3.87 (m, 2H), 3.59-3.50 (m, 2H), 3.06 (s, 2H), 2.96-2.87 (m, 5H), 2.59 (s, 3H), 2.23-2.13 (m, 5H), 2.08 (br. s., 2H), 1.32 (d, J=7.0 Hz, 6H). LC-MS (ES): m/z=656.2 [M+H]⁺; HPLC RT and purity: method E=5.53 min and 98.93% and method F=7.02 min and 98.95%.

Example 21 (Isomer 2): 5.41 mg, 6.88 μmol, 9.60% yield). ¹H NMR (400 MHz, D₂O) δ 8.70 (s, 1H), 8.49 (s, 1H), 7.64 (s, 1H), 7.35 (d, J=8.0 Hz, 1H), 7.14 (d, J=8.0 Hz, 1H), 5.80 (d, J=6.5 Hz, 2H), 4.12 (d, J=6.0 Hz, 2H), 4.05-3.91 (m, 4H), 3.61 (br. s., 1H), 3.52 (br. s., 1H), 3.06-2.99 (m, 2H), 2.99-2.85 (m, 4H), 2.60-2.50 (m, 4H), 2.35-2.20 (m, 2H), 2.16 (s, 3H), 2.02-2.06 (m, 2H), 1.33 (d, J=7.0 Hz, 6H). LC-MS (ES): m/z=656.2 [M+H]⁺; HPLC RT and purity: method E=4.84 min and 98.79% and method F=5.39 min and 98.64%.

### Example 22

### ((3-Methoxy-4-(phosphonooxy)benzoyl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

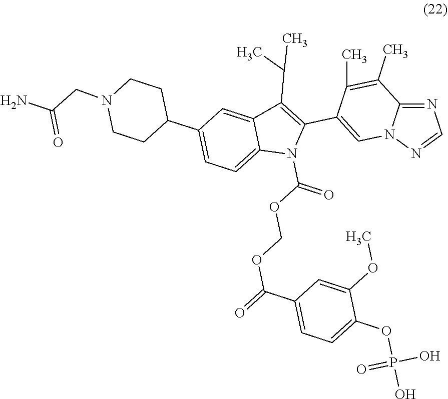 [SMILES: COc1cc(C(=O)OCOC(=O)n2c(-c3cn4ncnc4c(C)c3C)c(C(C)C)c3cc(C4CCN(CC(N)=O)CC4)ccc32)ccc1OP(=O)(O)O]

### Intermediate 22A: Chloromethyl 5-(1-(tert-butoxycarbonyl)piperidin-4-yl)-2-(7,8-dimethyl-[1,2,4]triazolo[1,5-a]pyridin-6-yl)-3-isopropyl-1H-indole-1-carboxylate

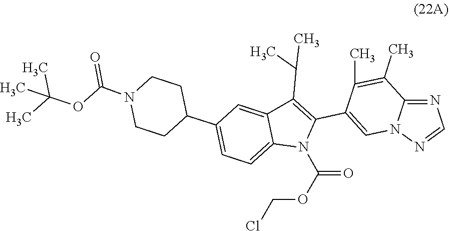 [SMILES: Cc1c(-c2c(C(C)C)c3cc(C4CCN(C(=O)OC(C)(C)C)CC4)ccc3n2C(=O)OCCl)cn2ncnc2c1C]

To a solution of tert-butyl 4-(2-(7,8-dimethyl-[1,2,4]triazolo[1,5-a]pyridin-6-yl)-3-isopropyl-1H-indol-5-yl)piperidine-1-carboxylate (1.0 g, 2.051 mmol) in THF (5 mL), was added 1 M solution of LiHMDS (3.08 mL, 3.08 mmol) in THF. The reaction mixture was stirred at −78° C. for 1 h under N2. Then chloromethyl chloroformate (0.529 g, 4.10 mmol) was added. The reaction mixture was stirred at −78° C. for 1 h under N2. The reaction mixture was partitioned between H₂O and EtOAc. The organic layer was washed with H₂O and brine, dried over anhydrous Na₂SO₄ and concentrated to yield the title compound (0.8 g, 1.379 mmol, 67.2% yield) as a light yellowish oil. ¹H NMR (400 MHz, DMSO-d₆) δ 8.83 (s, 1H), 8.60 (s, 1H), 8.08 (d, J=11.60 Hz, 1H), 7.66 (s, 1H), 7.32 (d, J=11.60 Hz, 1H), 5.92-5.83 (m, 2H), 4.12-4.07 (m, 2H), 2.87-2.72 (m, 4H), 2.56 (s, 3H), 2.08 (s, 3H), 1.83-1.75 (m, 2H), 1.59-1.51 (m, 2H), 1.41 (s, 9H), 1.28-1.23 (m, 6H). LC-MS (ES): m/z=580.5 [M+H]⁺.

### Intermediate 22B: Benzyl (4-((((5-(1-(tert-butoxycarbonyl)piperidin-4-yl)-2-(7,8-dimethyl-[1,2,4]triazolo[1,5-a]pyridin-6-yl)-3-isopropyl-1H-indole-1-carbonyl)oxy) methoxy)carbonyl)-2-methoxyphenyl) phosphate

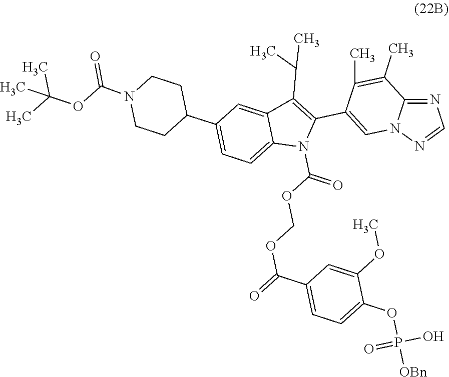 [SMILES: COc1cc(C(=O)OCOC(=O)n2c(-c3cn4ncnc4c(C)c3C)c(C(C)C)c3cc(C4CCN(C(=O)OC(C)(C)C)CC4)ccc32)ccc1OP(=O)(O)OCc1ccccc1]

To a stirred solution of chloromethyl 5-(1-(tert-butoxycarbonyl)piperidin-4-yl)-2-(7,8-dimethyl-[1,2,4]triazolo[1,5-a]pyridin-6-yl)-3-isopropyl-1H-indole-1-carboxylate (0.8 g, 1.379 mmol) in DMF (5.0 mL), were added DIPEA (1.204 mL, 6.90 mmol), TBAI (1.019 g, 2.76 mmol) and 4-((bis(benzyloxy)phosphoryl)oxy)-3-methoxybenzoic acid (1.181 g, 2.76 mmol). The homogeneous reaction mixture was stirred at room temperature for 16 h. The reaction mixture was partitioned between EtOAc and H₂O. The organic layer was washed with brine solution and dried over Na₂SO₄, filtered and concentrated under reduced pressure. The crude product was purified by reverse phase prep. HPLC (column: X-Bridge phenyl (250×19) mm; 5 micron; mobile phase A: 10 mM NH₄HCO₃ pH 9.5; mobile phase B: CH₃CN; flow rate: 20 mL/min; gradient (time (min)/% B): 0/40, 11.5/63). The fraction was concentrated using high vacuum at 30° C. The residue was dissolved in a mixture of CH₃CN and H₂O, frozen and lyophilized for 16 h to afford the title compound (180 mg, 0.200 mmol, 14.52% yield) as an off-white solid. ¹H NMR (400 MHz, DMSO-d₆) δ 8.74 (s, 1H), 8.37 (s, 1H), 8.15 (d, J=8.5 Hz, 1H), 7.69-7.61 (m, 2H), 7.36-7.30 (m, 2H), 7.30-7.24 (m, 4H), 7.24-7.17 (m, 2H), 5.97 (d, J=5.5 Hz, 1H), 5.84 (d, J=6.0 Hz, 1H), 4.79-4.73 (m, 2H), 4.12 (d, J=11.0 Hz, 1H), 3.79 (s, 3H), 3.22-3.11 (m, 4H), 2.84 (t, J=11.8 Hz, 1H), 2.31 (s, 3H), 1.97 (s, 3H), 1.82 (d, J=11.5 Hz, 2H), 1.66-1.51 (m, 2H), 1.43 (s, 9H), 0.97-0.89 (m, 6H). LC-MS (ES): m/z=881.5 [M+H]⁺.

### Intermediate 22C: ((4-(((Benzyloxy)(hydroxy)phosphoryl)oxy)-3-methoxybenzoyl)oxy) methyl 2-(7,8-dimethyl-[1,2,4]triazolo[1,5-a]pyridin-6-yl)-3-isopropyl-5-(piperidin-4-yl)-1H-indole-1-carboxylate, trifluoroacetate

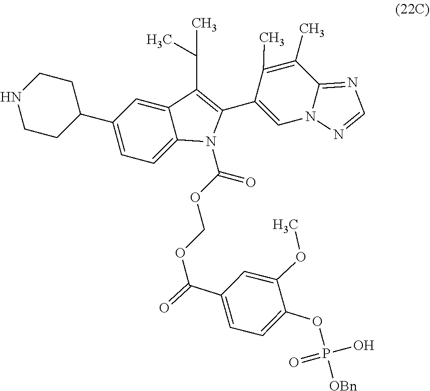 [SMILES: COc1cc(C(=O)OCOC(=O)n2c(-c3cn4ncnc4c(C)c3C)c(C(C)C)c3cc(C4CCNCC4)ccc32)ccc1OP(=O)(O)OCc1ccccc1]

To a stirred solution of ((4-(((benzyloxy)(hydroxy)phosphoryl)oxy)-3-methoxybenzoyl)oxy)methyl 5-(1-(tert-butoxycarbonyl)piperidin-4-yl)-2-(7,8-dimethyl-[1,2,4]triazolo[1,5-a]pyridin-6-yl)-3-isopropyl-1H-indole-1-carboxylate (0.2 g, 0.227 mmol) in CH₂Cl₂ (2.0 mL), CF₃CO₂H (0.2 mL, 2.60 mmol) was added at 0° C. The homogeneous reaction mixture was stirred at room temperature for 30 minutes. The reaction mixture was concentrated completely under reduced pressure to afford the product (0.19 g, 0.170 mmol, 74.8% yield) as a light brown oil. ¹H NMR (400 MHz, DMSO-d₆) δ 8.79 (s, 1H), 8.67 (br. s., 1H), 8.48-8.36 (m, 3H), 8.21 (d, J=9.0 Hz, 2H), 7.67 (br. s., 2H), 7.44-7.28 (m, 7H), 5.96 (d, J=6.0 Hz, 1H), 5.89 (d, J=5.5 Hz, 1H), 5.11-5.03 (m, 2H), 3.86 (s, 3H), 3.42 (d, J=12.0 Hz, 2H), 3.10-2.97 (m, 2H), 2.74-2.65 (m, 2H), 2.32 (s, 3H), 2.01 (s, 3H), 1.98-1.83 (m, 4H), 1.38-1.20 (m, 6H). LC-MS (ES): m/z=782.0 [M+H]⁺.

### Intermediate 22D: ((4-(((Benzyloxy)(hydroxy)phosphoryl)oxy)-3-methoxybenzoyl)oxy) methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

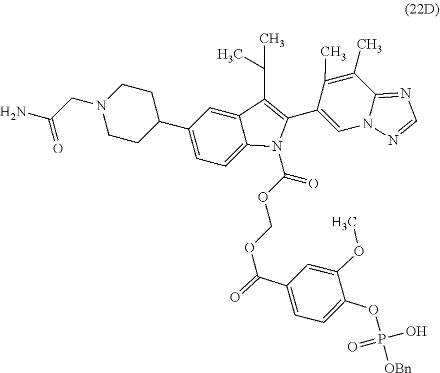 [SMILES: COc1cc(C(=O)OCOC(=O)n2c(-c3cn4ncnc4c(C)c3C)c(C(C)C)c3cc(C4CCN(CC(N)=O)CC4)ccc32)ccc1OP(=O)(O)OCc1ccccc1]

To a stirred solution of ((4-(((benzyloxy)(hydroxy)phosphoryl)oxy)-3-methoxybenzoyl)oxy)methyl 2-(7,8-dimethyl-[1,2,4]triazolo[1,5-a]pyridin-6-yl)-3-isopropyl-5-(piperidin-4-yl)-1H-indole-1-carboxylate (0.18 g, 0.201 mmol) in DMF (5.0 mL), were added Et₃N (0.084 mL, 0.603 mmol) and 2-bromoacetamide (0.042 g, 0.301 mmol) at 0° C. The homogeneous reaction mixture was stirred at room temperature for 16 h. The reaction mixture was partitioned between EtOAc and H₂O. The organic layer was dried over Na₂SO₄, filtered and concentrated under reduced pressure. The crude product was purified by reverse phase prep. HPLC (column: YMC Trait (250×20) mm, 5 micron; mobile phase A: 10 mM NH₄HCO₃ pH 9.5 in H₂O; mobile phase B: CH₃CN; flow rate: 18 mL/min; gradient: (time (min)/% B): 0/10, 20/70, 21/100). The fraction was concentrated using high vacuum at 30° C. The residue was dissolved in a mixture of CH₃CN and H₂O, frozen and lyophilized for 16 h to afford the title compound (80 mg, 0.093 mmol, 46.5% yield) as an off-white solid. ¹H NMR (300 MHz, DMSO-d₆) δ 9.75 (s, 1H), 8.76 (s, 1H), 8.38 (s, 1H), 8.16 (d, J=8.9 Hz, 1H), 7.72-7.59 (m, 3H), 7.42-7.14 (m, 9H), 5.97 (d, J=5.6 Hz, 1H), 5.85 (d, J=5.9 Hz, 1H), 4.78 (d, J=6.6 Hz, 2H), 3.79 (s, 3H), 3.26-3.16 (m, 2H), 2.91 (br. s., 2H), 2.76-2.65 (m, 4H), 2.31 (s, 3H), 2.08 (br. s., 2H), 2.03-1.89 (m, 5H), 1.38-1.19 (m, 6H). LC-MS (ES): m/z=839.0 [M+H]⁺.

### Example 22

To a stirred solution of ((4-(((benzyloxy)(hydroxy)phosphoryl)oxy)-3-methoxybenzoyl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (120 mg, 0.143 mmol) in dichloroethane (5.0 mL), were added CF₃CO₂H (1.0 mL, 12.98 mmol) and anisole (0.1 mL, 0.915 mmol). The reaction mixture was stirred at 55° C. for 1 h. The reaction mixture was concentrated completely under reduced pressure. The crude compound was stirred with Et₂O. The precipitated solid was dried under vacuum, dissolved in a mixture of CH₃CN and H₂O, frozen and lyophilized for 16 h to afford the title compound (90 mg, 0.115 mmol, 81% yield) as an off-white solid. ¹H NMR (300 MHz, DMSO-d₆) δ 8.76 (s, 1H), 8.37 (s, 1H), 8.17 (d, J=8.9 Hz, 1H), 8.00 (br. s., 1H), 7.75-7.65 (m, 2H), 7.51 (d, J=7.9 Hz, 1H), 7.41-7.25 (m, 3H), 5.96 (d, J=5.6 Hz, 1H), 5.87 (d, J=6.3 Hz, 1H), 3.92 (br. s., 2H), 3.53 (br. s., 3H), 3.18 (br. s., 2H), 2.96 (br. s., 2H), 2.67 (dd, J=14.5, 7.3 Hz, 2H), 2.32 (s, 3H), 2.12-1.93 (m, 7H), 1.36-1.20 (m, 6H). LC-MS (ES): m/z=749.2 [M+H]⁺; HPLC RT and purity: method A=8.040 min and 96.08% and method B=9.104 min and 97.20%.

### Example 23

### 1-(2-Amino-2-oxoethyl)-4-(2-(7,8-dimethyl-[1,2,4]triazolo[1,5-a]pyridin-6-yl)-3-isopropyl-1H-indol-5-yl)-1-(((methyl(2-(methylamino)ethyl)carbamoyl)oxy)methyl) piperidin-1-ium trifluoroacetate

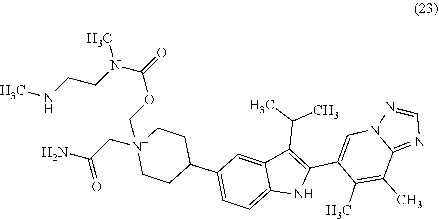 [SMILES: CNCCN(C)C(=O)OC[N+]1(CC(N)=O)CCC(c2ccc3[nH]c(-c4cn5ncnc5c(C)c4C)c(C(C)C)c3c2)CC1]

### Intermediate 23A: tert-Butyl (chloromethyl)ethane-1,2-diylbis(methylcarbamate)

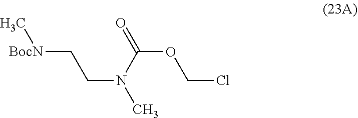 [SMILES: CN(CCN(C)C(=O)OC(C)(C)C)C(=O)OCCl]

To a stirred solution of tert-butyl methyl(2-(methylamino)ethyl)carbamate (3.00 g, 15.93 mmol) in CH₂Cl₂ (30 mL), were added pyridine (2.58 mL, 31.9 mmol) and chloromethyl chloroformate (2.84 mL, 31.9 mmol) at 0° C. The reaction mixture was stirred at 0° C. for 2 h. The reaction mixture was partitioned between H₂O and CH₂Cl₂. The organic layer was washed with brine solution, dried over anhydrous Na₂SO₄, filtered and concentrated under vacuum at 0° C. to afford the title compound (3.7 g, 13.18 mmol, 83% yield) as a light yellow oil. ¹H NMR (400 MHz, DMSO-d₆) δ 5.86 (d, J=9.0 Hz, 2H), 3.43-3.29 (m, 4H), 2.88 (s, 3H), 2.77 (br. s., 3H), 1.37 (s, 9H).

### Intermediate 23B: 1-(2-Amino-2-oxoethyl)-4-(2-(7,8-dimethyl-[1,2,4]triazolo[1,5-a]pyridin-6-yl)-3-isopropyl-1H-indol-5-yl)-1-(4,7,10,10-tetramethyl-3,8-dioxo-2,9-dioxa-4,7-diazaundecyl)piperidin-1-ium iodide

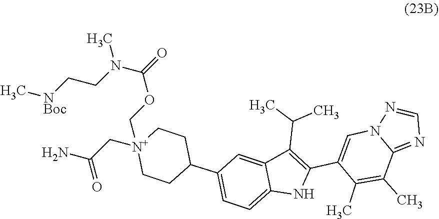 [SMILES: Cc1c(-c2[nH]c3ccc(C4CC[N+](COC(=O)N(C)CCN(C)C(=O)OC(C)(C)C)(CC(N)=O)CC4)cc3c2C(C)C)cn2ncnc2c1C]

To a solution of 2-(4-(2-(7,8-dimethyl-[1,2,4]triazolo[1,5-a]pyridin-6-yl)-3-isopropyl-1H-indol-5-yl)piperidin-1-yl)acetamide (0.500 g, 1.125 mmol) in CHCl₃ (15 mL), were added tert-butyl (chloromethyl)ethane-1,2-diylbis(methylcarbamate) (1.105 g, 3.94 mmol) and TBAI (1.454 g, 3.94 mmol). The reaction mixture was heated at 68° C. for 12 h. The reaction mixture was filtered through Celite bed which was washed with CHCl₃. The filtrate was concentrated in vacuo at 30° C. The crude product was purified using RP HPLC (column: SunFire OBD (250×30 mm), 5 micron; mobile phase A: 0.1% HCO₂H in H₂O; mobile phase B: CH₃CN; flow rate: 30 mL/min; gradient (T/% B): 0/20, 2/20, 12/40). The fraction was concentrated using high vacuum at 30° C. The residue was dissolved in a mixture of CH₃CN and H₂O, frozen and lyophilized for 12 h to yield a mixture of two isomers (135 mg, 17.4% yield) as an off-white solid. ¹H NMR (400 MHz, DMSO-d₆) δ 11.06 (s, 1H), 8.74 (s, 1H), 8.61-8.42 (m, 2H), 8.39 (br. s., 1H), 7.75 (br. s., 1H), 7.68-7.59 (m, 1H), 7.32-7.11 (m, 1H), 5.79-5.64 (m, 2H), 4.48-4.21 (m, 2H), 3.96-3.22 (m, 8H), 2.96-2.74 (m, 8H), 2.59 (s, 3H), 2.38-2.19 (m, 2H), 2.15 (s, 3H), 2.03 (d, J=13.8 Hz, 2H), 1.44-1.35 (m, 9H), 1.32 (d, J=7.0 Hz, 6H). LC-MS (ES): m/z=690.4 [M+H]⁺.

### Example 23

To a stirred solution of 1-(2-amino-2-oxoethyl)-4-(2-(7,8-dimethyl-[1,2,4]triazolo[1,5-a]pyridin-6-yl)-3-isopropyl-1H-indol-5-yl)-1-(4,7,10,10-tetramethyl-3,8-dioxo-2,9-dioxa-4,7-diazaundecyl)piperidin-1-ium iodide (0.130 g, 0.159 mmol) in dichloromethane (2 mL), was added CF₃CO₂H (0.123 mL, 1.592 mmol) at 0° C. The reaction mixture was stirred at 0° C. for 1 h. The reaction mixture was concentrated under vacuum at 30° C. The crude product was washed with Et₂O. The solid was dried under high vacuum at 30° C. The solid was dissolved in a mixture of CH₃CN and H₂O, frozen and lyophilized for 12 h to yield the title compound (0.105 g, 0.127 mmol, 80% yield) as an off-white solid. ¹H NMR (400 MHz, MeOH-d₄) δ 8.61 (s, 1H), 8.44 (s, 1H), 7.75-7.64 (m, 1H), 7.43-7.34 (m, 1H), 7.26-7.13 (m, 1H), 5.96-5.76 (m, 2H), 4.49-4.27 (m, 2H), 4.24-4.06 (m, 2H), 3.87-3.61 (m, 4H), 3.31-3.27 (m, 2H), 3.18-3.05 (m, 4H), 3.05-2.95 (m, 1H), 2.83-2.74 (m, 3H), 2.67 (s, 3H), 2.52-2.32 (m, 2H), 2.27 (s, 3H), 2.23-2.11 (m, 2H), 1.40 (d, J=7.0 Hz, 6H). LC-MS (ES): m/z=590.4 [M+H]⁺; HPLC RT and purity: method A=4.183 min and 98.85% and method B=4.471 min and 98.85%.

### Example 24

### Ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate

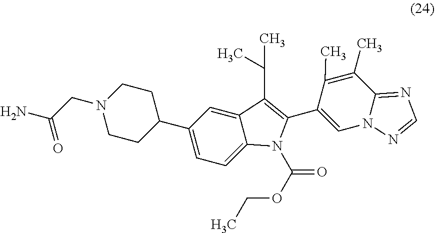 [SMILES: CCOC(=O)n1c(-c2cn3ncnc3c(C)c2C)c(C(C)C)c2cc(C3CCN(CC(N)=O)CC3)ccc21]

### Intermediate 24A: Ethyl 5-(1-(tert-butoxycarbonyl)piperidin-4-yl)-2-(7,8-dimethyl-[1,2,4]triazolo[1,5-a]pyridin-6-yl)-3-isopropyl-1H-indole-1-carboxylate

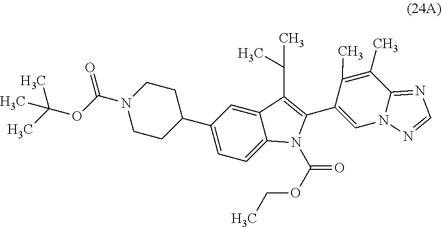 [SMILES: CCOC(=O)n1c(-c2cn3ncnc3c(C)c2C)c(C(C)C)c2cc(C3CCN(C(=O)OC(C)(C)C)CC3)ccc21]

To a solution of tert-butyl 4-(2-(7,8-dimethyl-[1,2,4]triazolo[1,5-a]pyridin-6-yl)-3-isopropyl-1H-indol-5-yl)piperidine-1-carboxylate (500 mg, 1.025 mmol) in DMF (5 mL), was added NaH (61.5 mg, 1.538 mmol). The reaction mixture was stirred at 0° C. for 30 min under N2. Next, ethyl chloroformate (0.117 mL, 1.230 mmol) was added. The reaction mixture was stirred at 0° C. to room temperature for 16 h under N2 and partitioned between H₂O and EtOAc. The organic layer was washed with H₂O and brine, dried over anhydrous Na₂SO₄ and concentrated. The crude material was purified by CombiFlash chromatography (60-120 silica gel; 10-50% EtOAc in petroleum ether as an eluent) to afford the title compound (290 mg, 51%) as a white solid. ¹H NMR (400 MHz, DMSO-d₆) δ 8.77 (s, 1H), 8.45 (s, 1H), 8.12 (d, J=8.5 Hz, 1H), 7.69-7.64 (m, 1H), 7.30 (dd, J=8.5, 1.5 Hz, 1H), 4.18-4.02 (m, 4H), 2.91-2.72 (m, 4H), 2.57 (s, 3H), 2.08 (s, 3H), 1.90-1.80 (m, 2H), 1.65-1.55 (m, 2H), 1.44 (s, 9H), 1.29 (t, J=7.5 Hz, 6H), 0.83 (t, J=7.0 Hz, 3H). LC-MS (ES): m/z=560.4 [M+H]⁺.

### Intermediate 24B: Ethyl 2-(7,8-dimethyl-[1,2,4]triazolo[1,5-a]pyridin-6-yl)-3-isopropyl-5-(piperidin-4-yl)-1H-indole-1-carboxylate trifluoroacetate

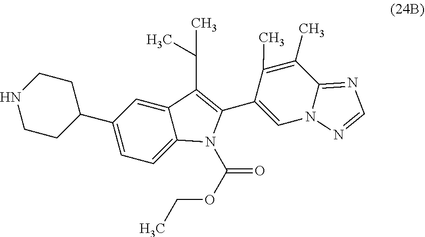 [SMILES: CCOC(=O)n1c(-c2cn3ncnc3c(C)c2C)c(C(C)C)c2cc(C3CCNCC3)ccc21]

To a solution of ethyl 5-(1-(tert-butoxycarbonyl)piperidin-4-yl)-2-(7,8-dimethyl-[1,2,4]triazolo[1,5-a]pyridin-6-yl)-3-isopropyl-1H-indole-1-carboxylate (300 mg, 0.536 mmol) in dry CH₂Cl₂ (1 mL) at 0° C. under nitrogen atmosphere, was added CF₃CO₂H (0.041 mL, 0.536 mmol). After being stirred at 0° C. for 1 h, the reaction mixture was concentrated in vacuo at 30° C. The residue was stirred with ether. The solvent was carefully decanted. The resultant solid was dried under vacuum to afford the title compound (298 mg, 0.520 mmol, 97% yield) as an off-white solid. ¹H NMR (400 MHz, DMSO-d₆) δ 8.79 (s, 1H), 8.61 (br. s., 1H), 8.47 (s, 1H), 8.35-8.37 (m, 1H), 8.17 (d, J=9.0 Hz, 1H), 7.66 (s, 1H), 7.28 (dd, J=8.5, 1.5 Hz, 1H), 4.18-4.03 (m, 2H), 3.42-3.45 (m, 2H), 3.12-2.96 (m, 3H), 2.77 (dt, J=14.1, 7.0 Hz, 1H), 2.57 (s, 3H), 2.08 (s, 3H), 2.06-1.98 (m, 2H), 1.97-1.83 (m, 2H), 1.30 (dd, J=11.0, 7.0 Hz, 6H), 0.83 (t, J=7.0 Hz, 3H). LC-MS (ES): m/z=460.4 [M+H]⁺.

### Example 24

To a solution of ethyl 2-(7,8-dimethyl-[1,2,4]triazolo[1,5-a]pyridin-6-yl)-3-isopropyl-5-(piperidin-4-yl)-1H-indole-1-carboxylate (298 mg, 0.648 mmol) in DMF (5 mL), were added 2-bromoacetamide (179 mg, 1.297 mmol) and Et₃N (0.271 mL, 1.945 mmol). The reaction mixture was stirred at room temperature for 1 h under N2. After being stirred at room temperature under nitrogen atmosphere for 1 h, the reaction mixture was diluted with EtOAc and washed with H₂O and brine, dried over anhydrous Na₂SO₄ and concentrated. The crude material was purified by reverse phase prep. HPLC (column: SunFire C18 (19×150 mm), 5 micron; mobile phase A: 10 mM NH₄OAc in H₂O; mobile phase B: CH₃CN; gradient: (time (min)/% B): 0/30, 15/80, 16/100, 18/100; flow rate: 20 mL/min) to afford the title compound (200 mg, 0.387 mmol, 59.7% yield) as a white solid. ¹H NMR (400 MHz, DMSO-d₆) δ 8.77 (s, 1H), 8.45 (s, 1H), 8.13 (d, J=8.5 Hz, 1H), 7.67 (s, 1H), 7.31 (dd, J=8.5, 1.5 Hz, 1H), 7.25 (br. s., 1H), 7.14 (br. s., 1H), 4.19-4.02 (m, 2H), 2.99-2.90 (m, 2H), 2.90 (s, 2H), 2.77 (quin, J=7.2 Hz, 1H), 2.70-2.60 (m, 1H), 2.57 (s, 3H), 2.26-2.17 (m, 2H), 2.08 (s, 3H), 1.92-1.75 (m, 4H), 1.30 (dd, J=8.8, 7.3 Hz, 6H), 0.83 (t, J=7.3 Hz, 3H). LC-MS (ES): m/z=517.4 [M+H]⁺; HPLC RT and purity: method E=5.08 min and 99.13% and method F=7.72 min and 99.09%.

### Example 25

### 2-(4-(2-(7,8-Dimethyl-[1,2,4]triazolo[1,5-a]pyridin-6-yl)-1-(hydroxymethyl)-3-isopropyl-1H-indol-5-yl)piperidin-1-yl)acetamide

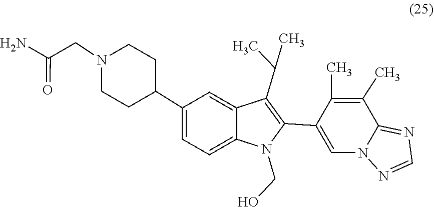 [SMILES: Cc1c(-c2c(C(C)C)c3cc(C4CCN(CC(N)=O)CC4)ccc3n2CO)cn2ncnc2c1C]

To a solution of 2-(4-(2-(7,8-dimethyl-[1,2,4]triazolo[1,5-a]pyridin-6-yl)-3-isopropyl-1H-indol-5-yl)piperidin-1-yl)acetamide (200 mg, 0.450 mmol) in DMF (2.0 mL), were added sodium hydride (54.0 mg, 1.350 mmol) and chloromethyl carbonochloridate (0.060 mL, 0.675 mmol). The reaction mixture was stirred at 0° C. for 1 h under N2. The reaction mixture was concentrated under high vacuum pump to yield a residue. The crude product was purified by reverse phase HPLC (column: YMC TRIAT (150×20) mm; 5 micron; mobile phase A: 10 mM NH₄OAc in H₂O; mobile phase B: CH₃CN; flow rate: 20 mL/min; gradient (T/% B): 0/20, 12/45). The fractions were concentrated under vacuum. The residue was dissolved in a mixture of CH₃CN and H₂O. The resulting mixture was frozen and lyophilized for 16 h to obtain the title compound (29.57 mg, 0.061 mmol, 38.9% yield) as a colorless solid. ¹H NMR (400 MHz, MeOH-d₄) δ 8.66 (s, 1H), 8.44 (s, 1H), 7.64 (d, J=1.5 Hz, 1H), 7.51 (d, J=8.5 Hz, 1H), 7.21 (dd, J=8.3, 1.8 Hz, 1H), 5.41 (d, J=11.0 Hz, 1H), 5.15 (d, J=11.5 Hz, 1H), 3.16-3.07 (m, 4H), 2.88 (quin, J=7.0 Hz, 1H), 2.74-2.64 (m, 4H), 2.39 (td, J=11.5, 3.0 Hz, 2H), 2.19 (s, 3H), 2.06-1.89 (m, 4H), 1.37 (dd, J=9.5, 7.0 Hz, 6H). LC-MS (ES): m/z=475.4 [M+H]⁺; HPLC RT and Purity: method A=8.75 min and 98.27% and method B=9.74 min and 98.25%.

### Solubility Evaluation in Aqueous Buffer

The solid powder of an individual prodrug was equilibrated in aqueous buffers of pH 1, 4 and 6.5 at 25° C. for about 24 hours. After equilibration, the excess solid was separated by filtration using 0.45 μm membrane filter and the filtrate was collected in suitable glass vial. The filtrate was diluted appropriately using respective buffers/diluent and analyzed by a reversed phase LC method with UV detection.

TABLE 1
Solubility data of the parent drug, Compound
A, and prodrugs in the pH range of 1.0-6.5^{a}

| | Solubility (mg/mL) at 25° C. | | |
|---|---|---|---|
| Ex. No. | pH 1 | pH 4 | pH 6.5 |
| Compound A | 1.56 | 0.05 | <0.01 |
| 1 | >1.74 | >1.28 | >1.34 |
| 2 | >1.17 | 0.24 | 0.43 |
| 3 | >2.43 | >2.68 | >2.44 |
| 4 | >1.64 | 0.91 | 0.66 |
| 5 | >2.84 | >2.15 | 1.19 |
| 6 | >1.32 | 0.52 | >1.22 |
| 7 | >1.49 | >1.21 | 0.34 |
| 8 | >0.86 | 0.03 | 0.86 |
| 9 | >1.14 | 0.85 | 1.34 |
| 10 | 0.43 | 0.1 | 0.12 |
| 11 | >1.03 | 0.007 | >1.02 |
| 14 | >1.51 | >0.98 | ND |
| 15 | >0.94 | >0.90 | 0.8 |
| 19 | >1.09 | 0.7 | >1.01 |
| 20 | >1.16 | >1.87 | >2.08 |
| 21 | >1.75 | 0.25 | 0.55 |
| 22 | >1.02 | 0.02 | 0.02 |
| 23 | >0.82 | >0.85 | ND |
| 24 | >1.39 | >2.93 | 0.1 |
| 25 | >1.41 | 1.28 | 0.01 |

^{a}In stability studies, 15% acetonitrile in buffer was used. Buffers, which were used, were 0.1N aqueous HCl (pH 1.0), acetate buffer (pH 4.0), and phosphate buffer (pH 6.5) at a concentration of 50 μg/mL.
ND = not determined.

Examples 1-11, 14-15, 19-21, and 23-25 invention were found to have greater solubility at pH 4 and/or pH 6.5 than Compound A. By improving solubility over a broader pH range, the compounds of the invention would have less variation in solubility in the stomach acid levels of the patient. Variations in the stomach pH may occur because of other medications or food that has been ingested. The data in Table 1 indicates that the tested compounds of the invention would be absorbed more uniformly independent of the pH of the stomach.

### Stability Evaluation in Aqueous Buffer

The stability study of an individual prodrug was conducted with about 50 μg/mL solutions in aqueous buffers of pH 1 (0.1 M HCl), 4 (acetate buffer) and 6.5 (phosphate buffer) at 37° C. for 24 hours. About 15% v/v acetonitrile was used in these pH buffers as a co-solvent to obtain clear solutions of prodrugs. The sample analysis was carried out using a generic reversed phase liquid chromatographic (LC) method with UV detection. The area percent (AP) loss of the prodrug peak was quantified as a function of time. The appearance of parent peak was identified from the chromatographic retention time. The half-life (t_{1/2}, h) of the prodrug was calculated by using a pseudo-first order equation from a AP loss vs time plot.

TABLE 2
Aqueous stability of the prodrugs in the pH range of 1.0-6.5^{a}

| | Stability (t_{1/2}, h) at 37° C. | | |
|---|---|---|---|
| Ex. No. | pH 1 | pH 4 | pH 6.5 |
| 1 | >500 | >500 | 218 |
| 2 | 248 | 283 | 267 |
| 3 | >500 | >500 | 99 |
| 4 | 231 | 257 | 231 |
| 5 | >500 | 233 | 4.5 |
| 6 | >500 | 415 | 108 |
| 7 | >500 | >500 | 193 |
| 8 | >500 | >500 | >500 |
| 9 | >500 | >500 | >500 |
| 10 | 347 | >500 | >500 |
| 11 | >500 | >500 | 154 |
| 12 | >500 | 99 | 1.6 |
| 13 | 459 | 99 | 2.5 |
| 14 | 179 | 85 | 1.6 |
| 15 | 347 | 139 | 39 |
| 16 | 80 | 7.5 | 0.1 |
| 17 | 2.3 | 2.1 | 65 |
| 18 | 6.4 | 2.9 | 6.3 |
| 19 | 157 | 207 | 65 |
| 20 | >500 | >500 | >500 |
| 21 | >500 | >500 | >500 |
| 22 | 400 | >500 | >500 |
| 23 | >500 | 9.6 | 0.1 |
| 24 | >500 | >500 | >500 |
| 25 | 179 | >500 | >500 |

Recombinant Intestinal Alkaline Phosphatase (rIALP) Assay

Examples 8 and 9 were evaluated in this assay. To assess the potential for generation of parent drug from the phosphate prodrugs, 100 μL of rIALP was dissolved in 100 mM Tris-HCl buffer (pH 7.4) and it was added to 100 μL of freshly prepared prodrug solution (10 mM) prepared in the same buffer. Incubations were performed in triplicate using 96-well plates (Waters, Milford, Mass.) at 37° C. in a shaking water bath (Julabo, Allentown, Pa.). The prodrug and enzyme solution were pre-warmed at 37° C. before the reaction was initiated by addition of substrate. The incubation was performed for 120 minutes with 100 μL aliquots taken at 0, 5, 10, 15, 30, 60, 90, and 120 minutes. The assays were terminated at designated time points by the addition of 200 μL of ice-cold acetonitrile containing internal standard followed by centrifugation at 4000 g for 4 minutes. The supernatant was transferred to a new 96-well plate for analysis by ultra-performance liquid chromatography combined with tandem mass spectrometry (UPLC-MS/MS). The t_{1/2} value for each prodrug/protein combination was determined from the natural logarithm of percent remaining versus time plots.

TABLE 3
Determination of half-life of phosphate
prodrugs in rat and human rIALP^{a}

| | t_{1/2} in rat rIALP | t_{1/2} in human rIALP |
|---|---|---|
| Ex. No. | (min) | (min) |
| 8 | <5 | <10 |
| 9 | <5 | <5 |

### Pharmacokinetic Evaluation of Select Prodrugs in Rats

The right jugular vein in SD rats was cannulated (JVC) by inserting a PE50 catheter with a silicon tip under isoflurane anesthesia and the rats were allowed to recover. The cannulated rats were fasted overnight with free access to water. Food was provided at 3 h post-dose.

Examples 3, 6, 8-10 and 15 (5 mg/kg or equivalent amount of 5 mg/kg of the parent compound) was dosed as a solution at a dose volume of 5 mL/kg in SD rats (N=3). The formulation used for these studies was 40% 20 mM citric acid solution, 10% ethanol, 45% PEG 300 and 5% Poloxamer 188. Blood samples (150 μL) were collected from the jugular vein catheter at 0.25, 0.5, 1, 3, 5, 7 and 24 h post-dose and the catheter was flushed with heparinized saline after each sample collection. Blood samples were collected into tubes containing 2% K2EDTA along with add 30 μL of PMSF solution during the sample collection (blood) and immediately centrifuged at 10000×g for 5 min at 4° C. to collect the plasma. An aliquot of separated plasma (30 μL) was immediately quenched with 150 μL of ACN containing ritonavir (200 nM) as the internal standard, in the case of prodrug. Plasma samples were analyzed using an ultra-performance liquid chromatography combined with a tandem mass spectrometry (UPLC/MS/MS).

### Sample Preparation for Bioanalysis

Separate calibration curves of parent and prodrug were prepared in rat plasma by serial dilution method for the concentration range of 0.169 to 10000 nM. A 30 μL aliquot of plasma standards/study samples (extracted from blood containing PMSF) was online quenched with 150 μL of CH₃CN containing internal standard (ritonavir: 200 nM). The quenched samples were filtered through a Millipore Solvinert hydrophilic plate. The samples were then centrifuged at 3700 rpm for 3 min at 4° C. The eluent thus obtained (4 μL) was injected onto the mass spectrometer for analysis. UPLC-MS/MS method: Waters Acquity UPLC™ (ultra-performance liquid chromatography) Integrated System (Milford, USA) was used to analyse samples on a reverse phase column (Waters: BEH C18, 1.7 microns, 2.1*50 mm) maintained at 40±2° C. Samples were analysed in a shallow gradient (solvent A: 0.1% v/v formic acid in 10 mM ammonium formate; solvent B: 0.1% v/v formic acid in CH₃CN) at a flow rate of 0.5 mL/min, with a total run time of 3 min. A linear gradient was used for the separation, wherein mobile phase B started with 10% and went up to 90% in 2.6 min. In the next 0.2 min, mobile phase B concentration came back to 10%.

Quantitation was achieved by MS/MS detection in positive ion multiple reaction monitoring (MRM) mode using API 4000 Q-Trap mass spectrometer (Applied Biosystems, MDS Sciex Toronto, Canada) equipped with an electrospray ionization (ESI) source at a capillary voltage of 5.5 kV and a source temperature of 500° C.

Quadrupole Q1 and Q3 were set at unit resolution. The analytical data were acquired and processed by Analyst software (version 1.5.0). The linearity of calibration standards was assessed by subjecting the spiked concentrations and the respective peak area ratios using weighted 1/X² linear least-squares regression analysis. The batch was accepted if the percentage coefficient of variation of internal standard was <15%. The accuracy of all calibration standards was within 80-120%.

### Data Analysis

The pharmacokinetic parameters were calculated by non-compartmental analysis using KINETICA™ software (Version 5.1) ThermoFisher Scientific Corporation, Philadelphia. For the oral group, the peak concentration (Cₘₐₓ) and time for Cₘₐₓ (Tₘₐₓ) were recorded directly from the experimental observations. The area under the plasma concentration-time curve (AUC₀₋ₜ) up to the last measurable concentration (Cₗₐₛₜ) was calculated using the mixed log-linear trapezoidal rule.

TABLE 4
Pharmacokinetic data of the parent obtained following
oral administration of the parent or prodrugs

| | Dose^{a} | Cₘₐₓ | Tₘₐₓ | C_{24 h} | AUCₗₐₛₜ |
|---|---|---|---|---|---|
| Ex. No. | (mg/kg) | (nM) | (h) | (nM) | (nM.h) |
| Compound A | 5 | 2944 | 3 | 457 | 30948 |
| 3 | 5 | 34 | 5 | 11 | 454 |
| 6 | 5 | 2757 | 3.7 | 477 | 34757 |
| 8 | 5 | 2199 | 3 | 284 | 24518 |
| 9 | 5 | 1091 | 5 | 159 | 12345 |
| 10 | 5 | 952 | 4.33 | 165 | 11849 |
| 15 | 5 | 669 | 3.7 | 85 | 6894 |

^{a}In the case of prodrugs, equivalent dose of the parent was used except in the case of Example 10.

### Crystal Structure Determination

### Crystallization Details:

Example 7: Single crystals were solved as a mixed solvate hydrate or a hydrated TFA salt were prepared from a saturated solution of Example 7 in methanol kept at ˜25° C. in an open dram vial. Transparent colorless block shaped crystals were obtained after complete evaporation of the solvent.

Crystal Data for Example 7 (M=789.78 g/mol): triclinic, space group P-1 (no. 2), a=9.4971(8) Å, b=13.0969(13) Å, c=18.0138(16) Å, α=89.263(6°), β=81.145(5°), γ=75.206(5°), V=2139.7(3) Å³, Z=2, T=296.15 K, μ(MoKα)=0.103 mm⁻¹, Dcalc=1.226 g/cm³, 23363 reflections measured (2.288°≤2Θ≤49.998°), 7143 unique (Rᵢₙₜ=0.0963, R_{sigma}=0.1644) which were used in all calculations. The final R₁ was 0.2685 (I>2σ(I)) and wR₂ was 0.5907 (all data). A few high intensity q-peaks were seen in the structure which were not modelled for simplicity of the structure model. These q-peaks probably correspond to disordered water/solvent molecules which are not assigned in the current model.

Example 8: Single crystals were prepared from saturated solution of Example 8 in a mixture of solvents (ethanol:methanol:acetonitrile˜1:1:1) at −25° C. in an open dram vial. Transparent light yellow block shaped crystals were obtained after complete evaporation of the solvent.

Crystal Data for Example 8 (M=592.11 g/mol): triclinic, space group P-1 (no. 2), a=9.8852(16) Å, b=11.0560(16) Å, c=29.357(4) Å, α=85.118(8°), β=85.506(9°), γ=84.663(8°), V=3174.9(8) Å³, Z=4, T=296.15 K, μ(MoKα)=0.231 mm⁻¹, Dcalc=1.239 g/cm³, 46825 reflections measured (2.792°≤2Θ≤49.998°), 11169 unique (Rᵢₙₜ=0.2467, R_{sigma}=0.3114) which were used in all calculations. The final R₁ was 0.1471 (I>2σ(I)) and wR₂ was 0.4877 (all data).

A few high intensity q-peaks were seen in the structure which seemed to correspond to highly disordered solvent molecule (probably ethyl acetate), solvent mask was used during the refinement for simplicity of the structure model. Also, one of the two API molecules in the asymmetric unit was observed to be disordered.

### Single Crystal X-Ray Diffraction:

Suitable quality single crystal was mounted on a HAMPTON CryoLoop using paratone oil in open condition. Data were collected on Bruker AXS SMART APEX II CCD diffractometer at room temperature (˜296 K). Data integration and reduction were carried out with SAINT v7.68A (Bruker, 2009) and absorption correction was performed by SADABS-2008/1 (Bruker, 2008) in the Bruker software suite. The structure was solved using intrinsic phasing or direct methods with SHELXS-97 (Sheldrick 2008) and refined using SHELXL-97 (version 2014/7; Sheldrick 2014) program in SHELXTL. OLEX2 was used for Structure solution, refinements, visualization and CIF and diagrams preparation. MERCURY was used for the analysis of intermolecular interactions. All the hydrogen atoms were fixed geometrically and refined isotropically.

What is claimed is:

1. A compound of Formula (I), Formula (II), Formula (III), and Formula (IV): 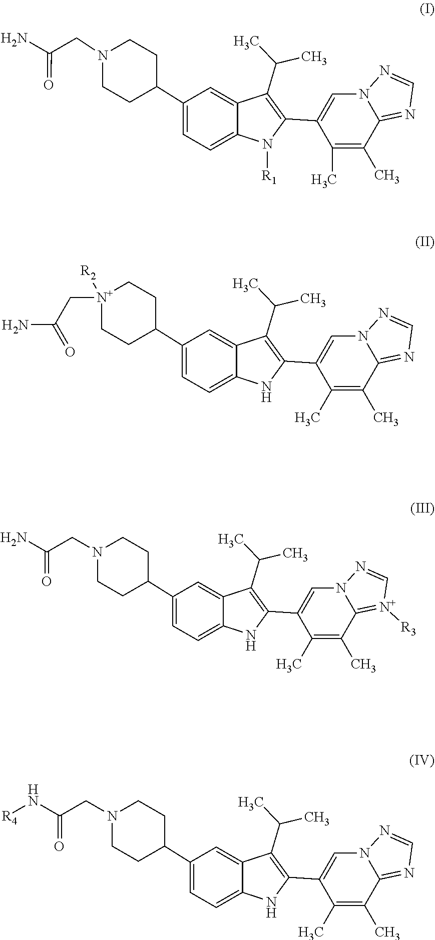
or a salt thereof, wherein:
R₁ is —CH₂OH, —C(O)O(C₁₋₄ alkyl), —C(O)CH₂NRₓRₓ, —C(O)(CH₂)₁₋₃OP(O)(OH)₂, —C(O)CH₂NRₓC(O)OCH₂OP(O)(OH)₂, —C(O)OCH₂(pyrrolidinyl), —C(O)OCH₂(piperidinyl), —C(O)OCHRₓOC(O)(aminocyclopropyl), —C(O)OCH(CH₃)OC(O)(aminocyclopropyl), —C(O)OCH₂OP(O)(OH)₂, —P(O)(OH)₂, —SCH₂CH(NH₂)C(O)OH, 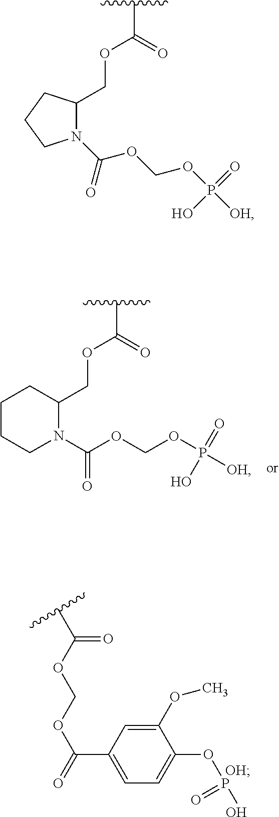
R₂ and R₃ are independently —CH₂OP(O)(OH)₂, —CH₂OC(O)NRₓCH₂CH₂NRₓRₓ, —CH₂OC(O)NRₓCH₂CH₂OP(O)(OH)₂, or 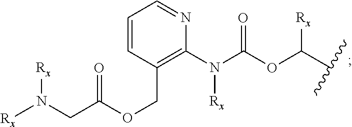
and
R₄ is —P(O)(OH)₂; and
each Rₓ is independently hydrogen or —CH₃.

2. The compound according to claim 1, or a salt thereof, wherein:
R₁ is —CH₂OH, —C(O)CH₂NH(CH₃), —C(O)CH₂CH₂CH₂OP(O)(OH)₂, —C(O)CH₂N(CH₃)C(O)OCH₂OP(O)(OH)₂, —C(O)OCH₂CH₃, —C(O)OCH₂(pyrrolidinyl), —C(O)OCH₂(piperidinyl), —C(O)OCH₂OC(O)(aminocyclopropyl), —C(O)OCH(CH₃)OC(O)(aminocyclopropyl), —C(O)OCH₂OP(O)(OH)₂, —P(O)(OH)₂, —SCH₂CH(NH₂)C(O)OH, 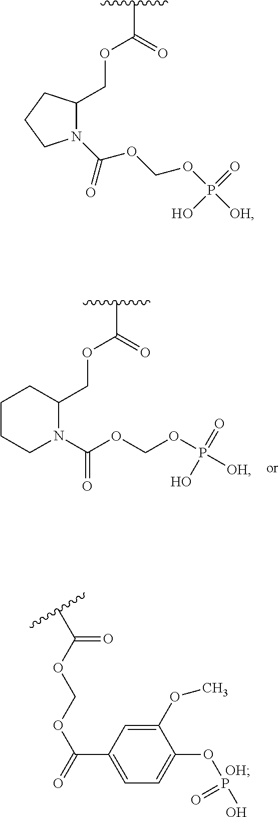
R₂ is —CH₂OP(O)(OH)₂, —CH₂OC(O)N(CH₃)CH₂CH₂NH(CH₃), —CH₂OC(O)N(CH₃)CH₂CH₂OP(O)(OH)₂, 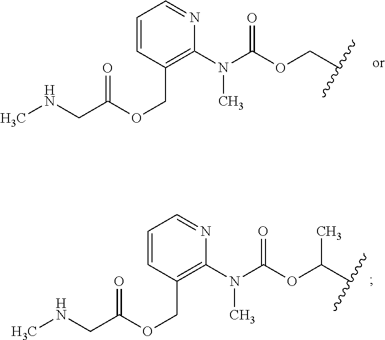 [SMILES: *COC(=O)N(C)c1ncccc1COC(=O)CNC ; *C(C)OC(=O)N(C)c1ncccc1COC(=O)CNC]
R₃ is —CH₂P(O)(OH)₂, —CH₂OC(O)N(CH₃)CH₂CH₂OP(O)(OH)₂, or 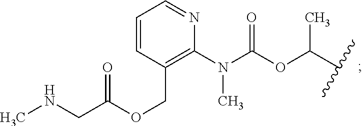 [SMILES: *C(C)OC(=O)N(C)c1ncccc1COC(=O)CNC]
and
R₄ is —P(O)(OH)₂.

3. The compound according to claim 1, or a salt thereof, having the structure of Formula (I).

4. The compound according to claim 1 or a salt thereof, having the structure of Formula (II).

5. The compound according to claim 1 or a salt thereof, having the structure of Formula (III).

6. The compound according to claim 1 or a salt thereof, having the structure of Formula (IV).

7. (canceled)

8. A pharmaceutical composition comprising a compound according to claim 1 or a pharmaceutically-acceptable salt thereof, and a pharmaceutically acceptable carrier.

9. (canceled)

10. (canceled)

11. The compound according to claim 1 or a salt thereof, wherein said compound is:
(S)-piperidin-2-ylmethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (1);
(S)-(1-(((phosphonooxy)methoxy)carbonyl)piperidin-2-yl)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (2);
(S)-pyrrolidin-2-ylmethyl 5-(1-(2-amino-2-oxoethyl) piperidin-4-yl)-2-(7,8-dimethyl-[1,2,4]triazolo[1,5-a]pyridin-6-yl)-3-isopropyl-1H-indole-1-carboxylate (3);
(S)-(1-(((phosphonooxy)methoxy)carbonyl)pyrrolidin-2-yl) methyl 5-(1-(2-amino-2-oxoethyl)piperidin-4-yl)-2-(7,8-dimethyl-[1,2,4]triazolo[1,5-a] pyridin-6-yl)-3-isopropyl-1H-indole-1-carboxylate (4);
2-(4-(2-(7,8-dimethyl-[1,2,4]triazolo[1,5-a] pyridin-6-yl)-3-isopropyl-1-(methylglycyl)-1H-indol-5-yl) piperidin-1-yl)acetamide ditrifluoroacetate (5);
1-((1-aminocyclopropane-1-carbonyl) oxy)ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate ditrifluoroacetate (6-7);
4-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-4-oxobutyl dihydrogen phosphate (10);
S-(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)-L-cysteine (11);
((1-aminocyclopropane-1-carbonyl)oxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate trifluoroacetate (15);
(phosphonooxy)methyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (16);
(5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)phosphonate (18);
(phosphonooxy)methyl (2-(5-(1-(2-amino-2-oxoethyl)piperidin-4-yl)-2-(7,8-dimethyl-[1,2,4]triazolo[1,5-a] pyridin-6-yl)-3-isopropyl-1H-indol-1-yl)-2-oxoethyl)(methyl)carbamate (19);
((3-methoxy-4-(phosphonooxy) benzoyl)oxy)methyl 5-(1-(2-amino-2-oxoethyl) piperidin-4-yl)-2-(7,8-dimethyl-[1,2,4]triazolo[1,5-a]pyridin-6-yl)-3-isopropyl-1H-indole-1-carboxylate (22);
ethyl 5-(1-(2-amino-2-oxoethyl)piperidin-4-yl)-2-(7,8-dimethyl-[1,2,4]triazolo[1,5-a]pyridin-6-yl)-3-isopropyl-1H-indole-1-carboxylate (24); or
2-(4-(2-(7,8-dimethyl-[1,2,4]triazolo[1,5-a]pyridin-6-yl)-1-(hydroxymethyl)-3-isopropyl-1H-indol-5-yl)piperidin-1-yl)acetamide (25).

12. The compound according to claim 1 or a salt thereof, wherein said compound is:
1-(2-amino-2-oxoethyl)-4-(2-(7,8-dimethyl-[1,2,4]triazolo[1,5-a] pyridin-6-yl)-3-isopropyl-1H-indol-5-yl)-1-((phosphonooxy)methyl)piperidin-1-ium trifluoroacetate (8);
1-(2-amino-2-oxoethyl)-4-(2-(7,8-dimethyl-[1,2,4]triazolo[1,5-a] pyridin-6-yl)-3-isopropyl-1H-indol-5-yl)-1-(((methyl(3-(((methylglycyl)oxy)methyl) pyridin-2-yl)carbamoyl)oxy)methyl)piperidin-1-ium ditrifluoroacetate (12);
1-(2-amino-2-oxoethyl)-4-(2-(7,8-dimethyl-[1,2,4]triazolo[1,5-a]pyridin-6-yl)-3-isopropyl-1H-indol-5-yl)-1-(1-((methyl(3-(((methylglycyl)oxy)methyl)pyridin-2-yl)carbamoyl)oxy)ethyl) piperidin-1-ium ditrifluoroacetate (14);
1-(2-amino-2-oxoethyl)-4-(2-(7,8-dimethyl-[1,2,4]triazolo[1,5-a]pyridin-6-yl)-3-isopropyl-1H-indol-5-yl)-1-(((methyl(2-(phosphonooxy)ethyl)carbamoyl)oxy)methyl)piperidin-1-ium trifluoroacetate (20); or
1-(2-amino-2-oxoethyl)-4-(2-(7,8-dimethyl-[1,2,4]triazolo[1,5-a]pyridin-6-yl)-3-isopropyl-1H-indol-5-yl)-1-(((methyl(2-(methylamino)ethyl)carbamoyl)oxy)methyl)piperidin-1-ium trifluoroacetate (23).

13. The compound according to claim 1 or a salt thereof, wherein said compound is:
6-(5-(1-(2-amino-2-oxoethyl)piperidin-4-yl)-3-isopropyl-1H-indol-2-yl)-7,8-dimethyl-1-((phosphonooxy) methyl)-[1,2,4]triazolo[1,5-a]pyridin-1-ium trifluoroacetate (9);
6-(5-(1-(2-amino-2-oxoethyl)piperidin-4-yl)-3-isopropyl-1H-indol-2-yl)-7,8-dimethyl-1-(((methyl(3-(((methylglycyl)oxy)methyl)pyridin-2-yl)carbamoyl)oxy) methyl)-[1,2,4]triazolo[1,5-a]pyridin-1-ium tritrifluoroacetate (13); or
6-(5-(1-(2-amino-2-oxoethyl)piperidin-4-yl)-3-isopropyl-1H-indol-2-yl)-7,8-dimethyl-1-(((methyl(2-(phosphonooxy)ethyl)carbamoyl)oxy)methyl)-[1,2,4]triazolo[1,5-a]pyridin-1-ium trifluoroacetate (21).

14. The compound according to claim 1 or a salt thereof, wherein said compound is 5-(1-(2-amino-2-oxoethyl)piperidin-4-yl)-2-(7,8-dimethyl-[1,2,4]triazolo[1,5-a]pyridin-6-yl)-3-isopropyl-1H-indol-1-yl)phosphonic acid (17).

15. A method of treating an autoimmune disease or a chronic inflammatory disease, comprising administering to a mammalian patent a compound according to claim 1 or a pharmaceutically acceptable salt thereof, wherein said autoimmune disease or chronic inflammatory disease is selected from systemic lupus erythematosus (SLE), rheumatoid arthritis, multiple sclerosis (MS), and Sjögren's syndrome.
